(19) United States
(12) Patent Application Publication
(10) Pub. No.: US 2025/0282845 A1
(43) Pub. Date: Sep. 11, 2025

(54) FUSION PROTEINS COMPRISING CHIMERIC ANTIGEN RECEPTORS AND IL-15

(71) Applicant: GC CELL CORPORATION, Yongin-si Gyeonggi-do (KR)

(72) Inventors: Yu Kyeong HWANG, Bojeong-dong (KR); Sungyoo CHO, Bojeong-dong (KR); Hoyong LIM, Bojeong-dong (KR); Miyoung JUNG, Bojeong-dong (KR); Hansol KIM, Bojeong-dong (KR); Hyun Ah KIM, Bojeong-dong (KR)

(21) Appl. No.: 18/285,631

(22) PCT Filed: Apr. 7, 2022
(86) PCT No.: PCT/KR2022/095077

§ 371 Date: Oct. 4, 2023

(30) Foreign Application Priority Data
Apr. 8, 2021 (KR) 10-2021-0045765

(51) Int. Cl. C07K 14/705; A61K 40/15; A61K 40/31; A61K 40/35; A61K 40/42; C07K 14/54; C07K 16/28; C07K 16/32; C12N 5/0783

(52) U.S. Cl. CPC C07K 14/70575; A61K 40/15; A61K 40/31; A61K 40/35; A61K 40/4205; A61K 40/4211; C07K 14/5443; C07K 16/2803; C07K 16/32; C12N 5/0646; C07K 2317/53; C07K 2317/622; C07K 2317/73; C07K 2319/02; C07K 2319/03; C12N 2510/00

(57) ABSTRACT

Provided herein, amongst other things, are polynucleotides comprising a) a chimeric antigen receptor (CAR) comprising: an extracellular antigen binding domain comprising an antibody or antigen binding fragment thereof; a transmembrane region; and an intracellular signaling region comprising an OX40L intracellular signaling domain; and b) a nucleic acid encoding an IL-15.

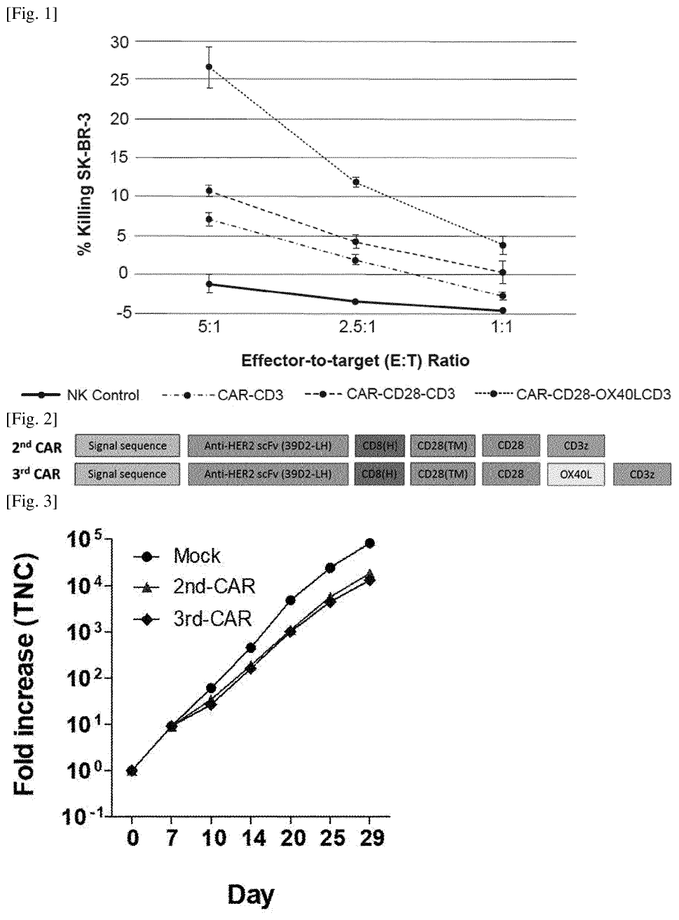

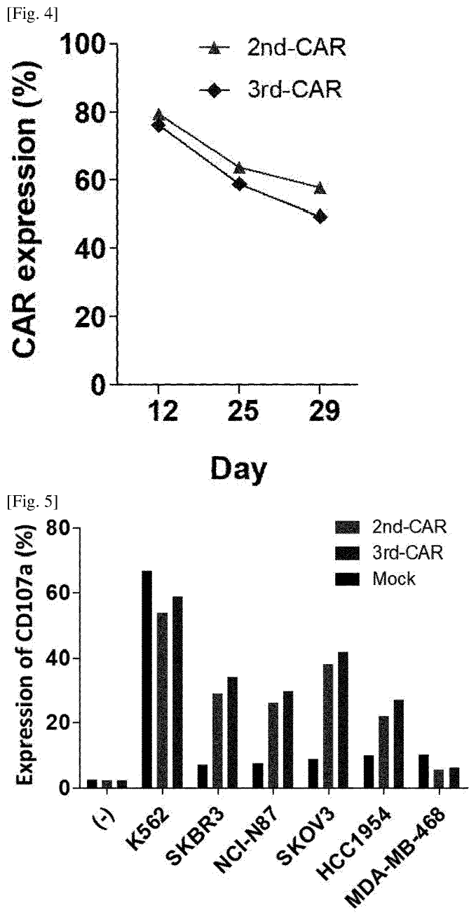

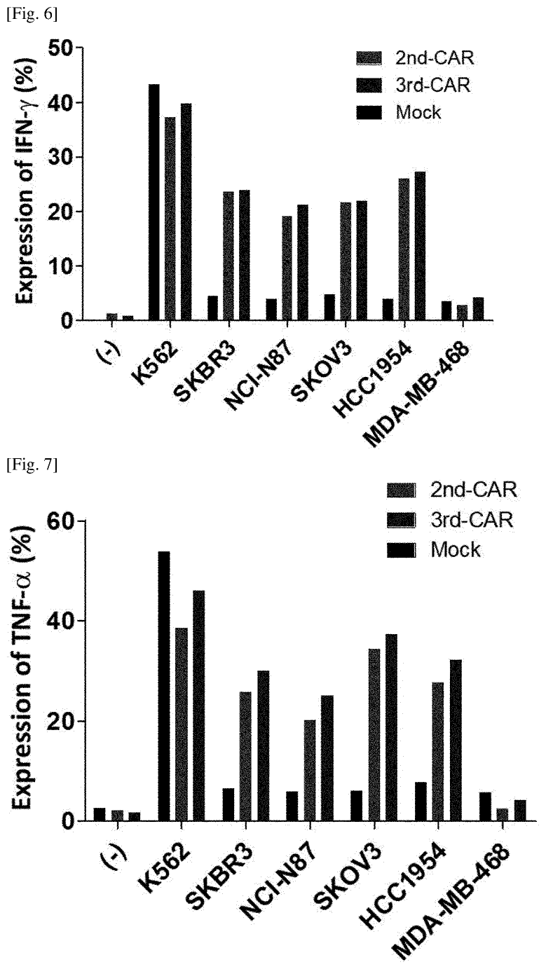

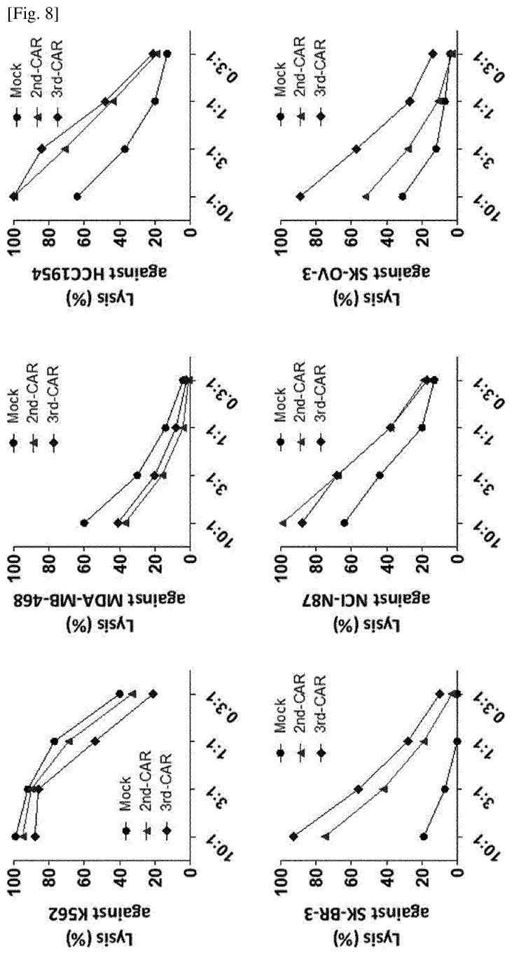

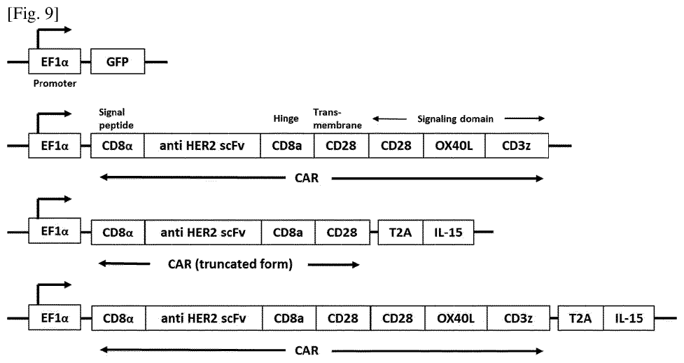

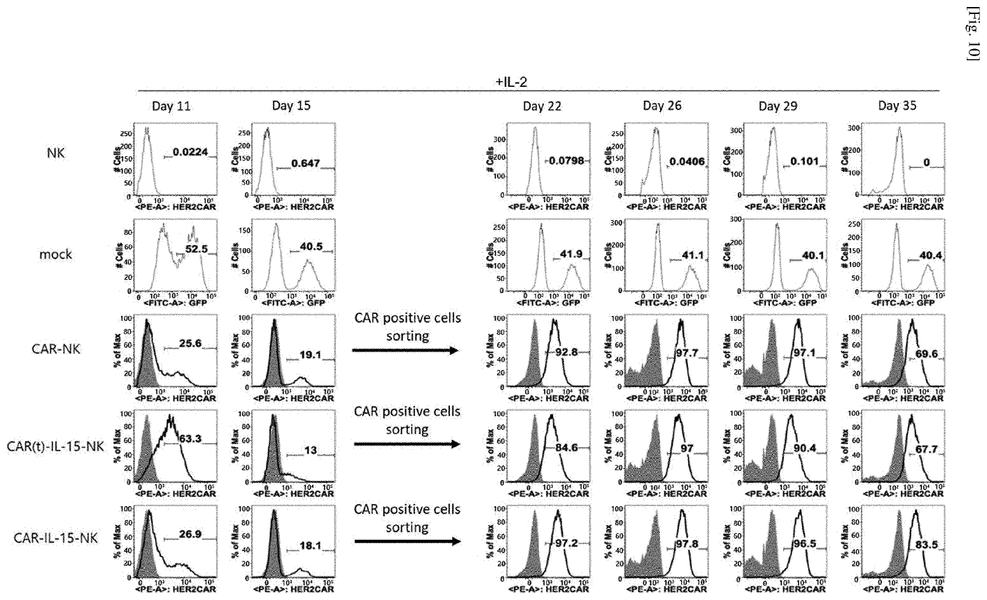

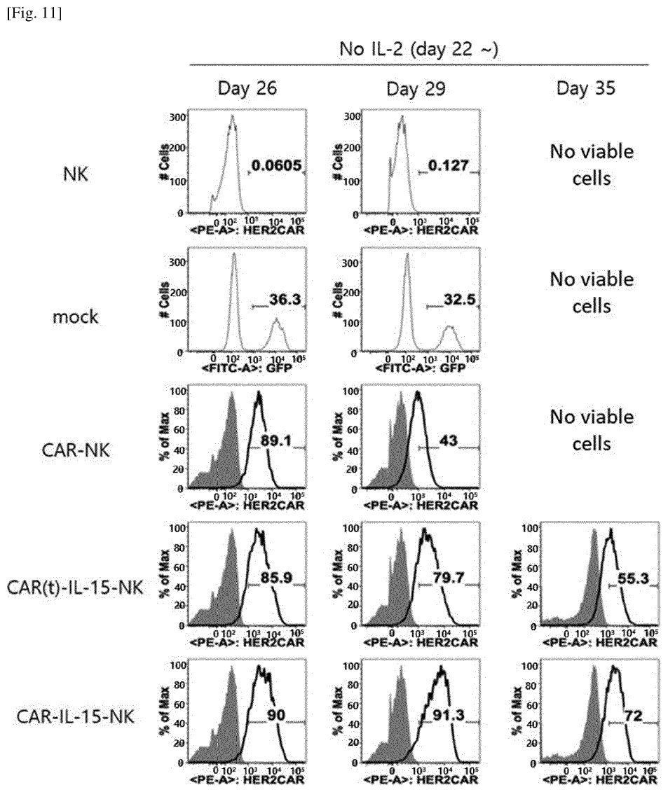

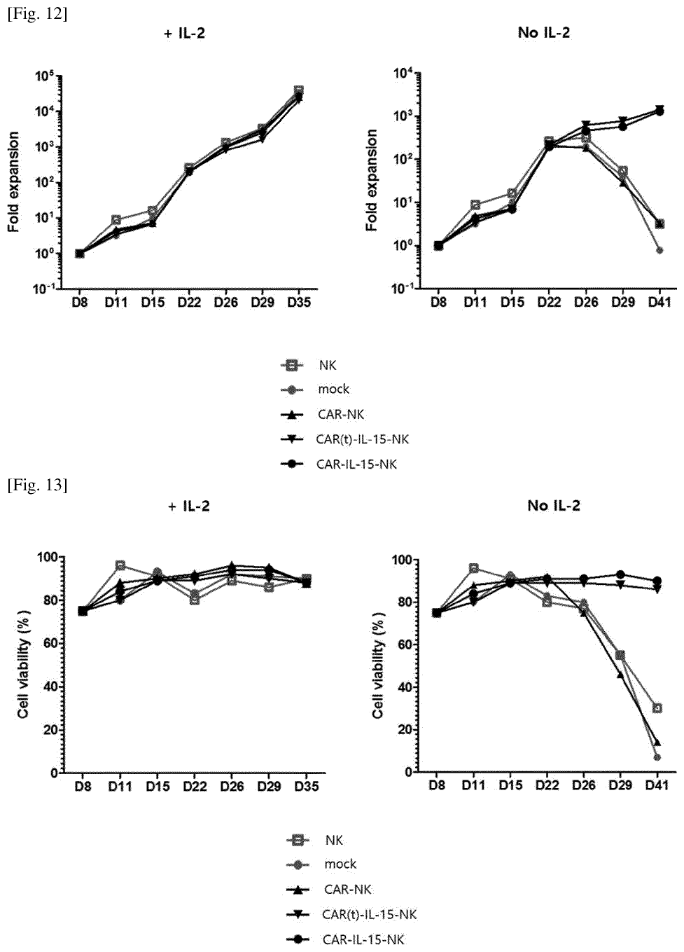

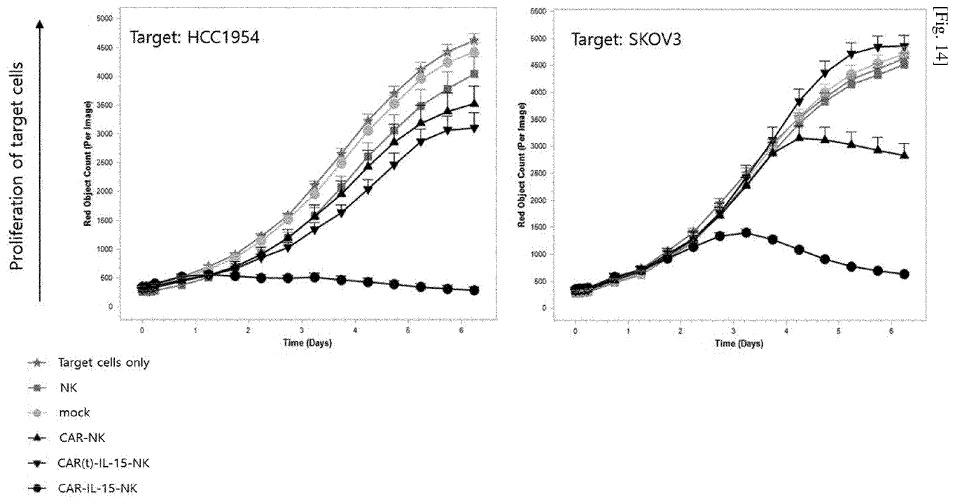

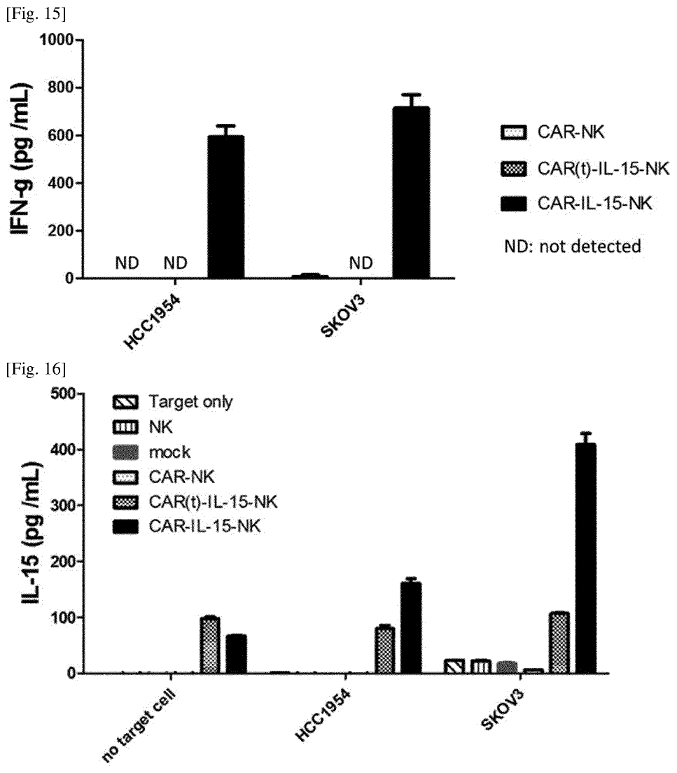

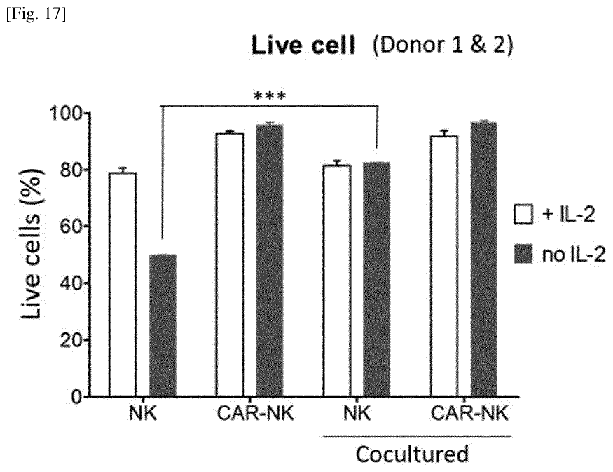

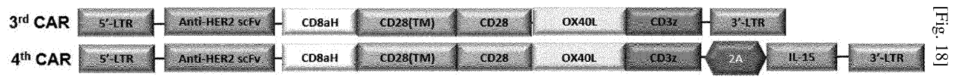

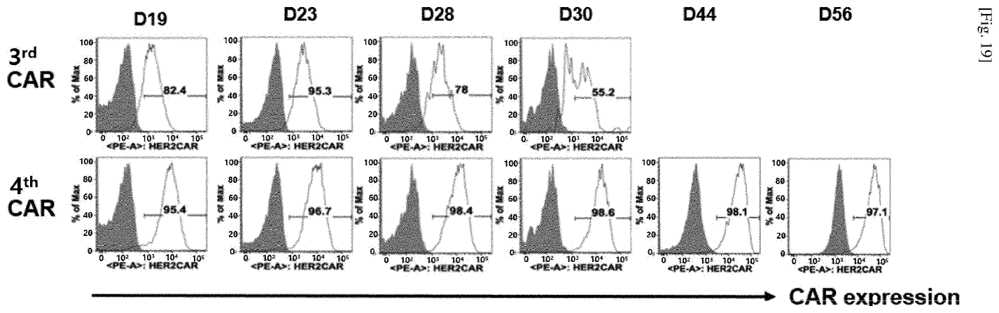

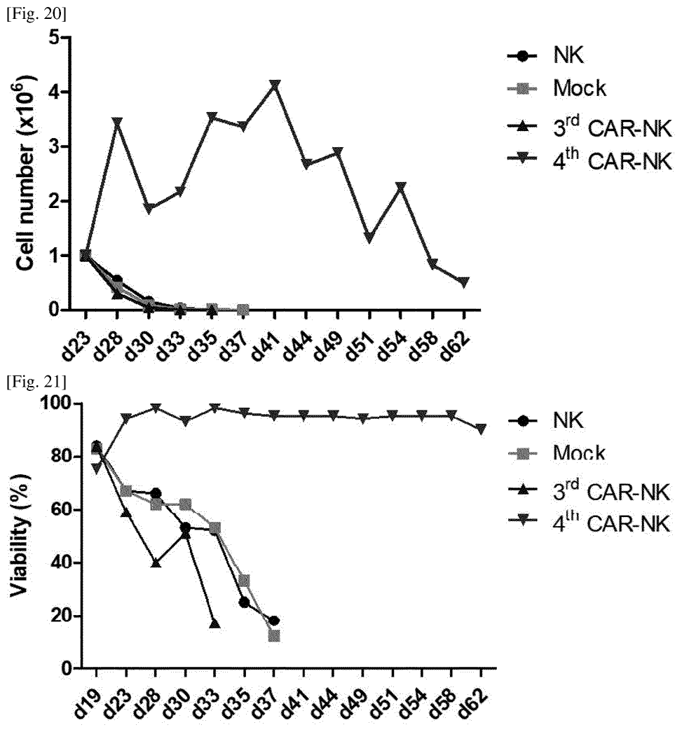

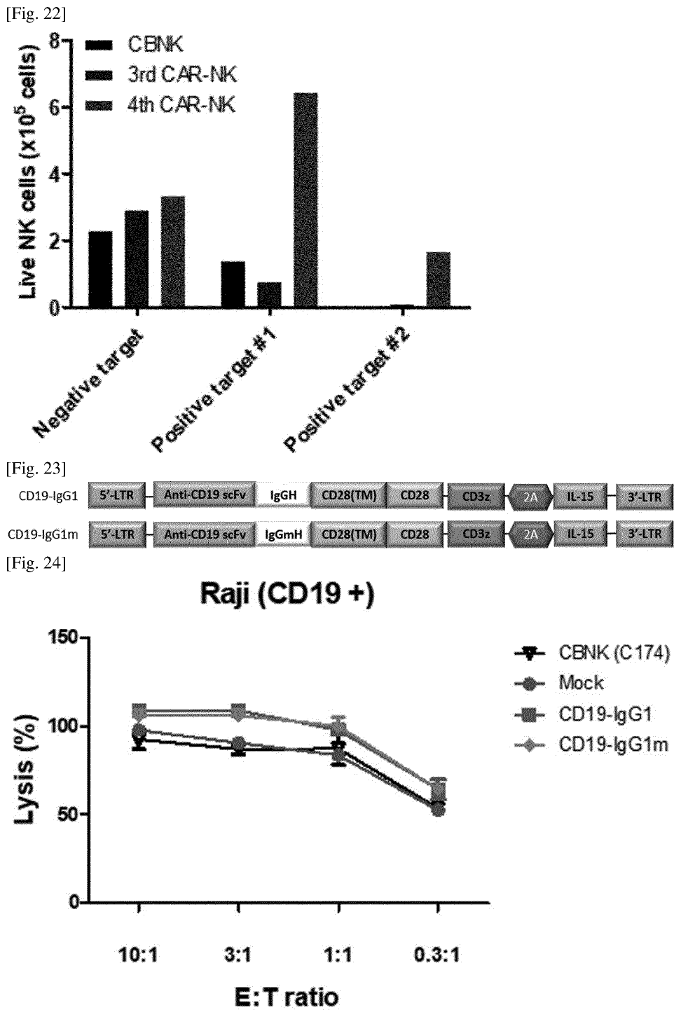

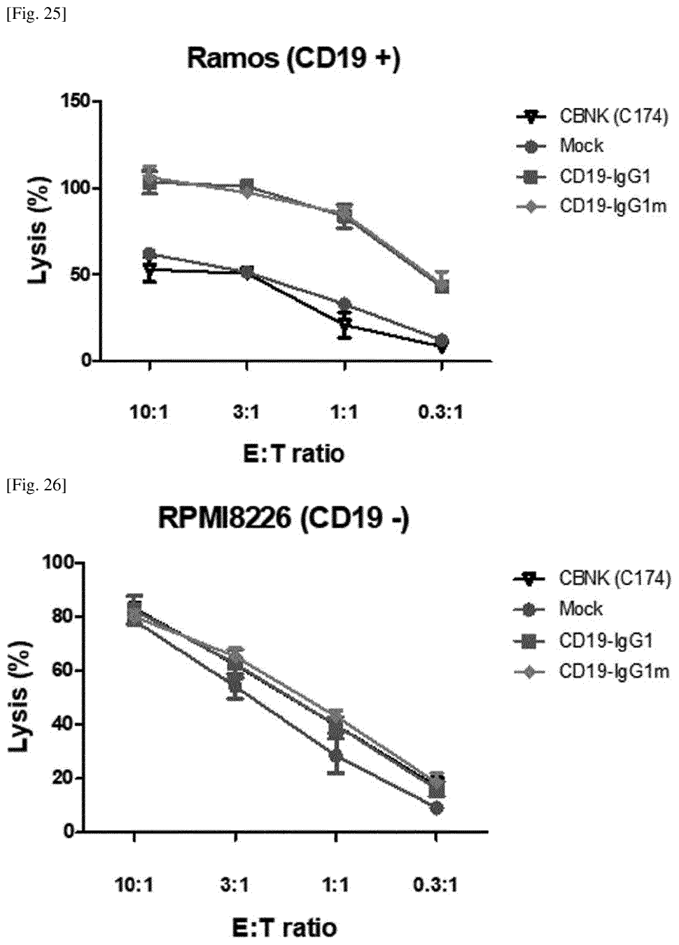

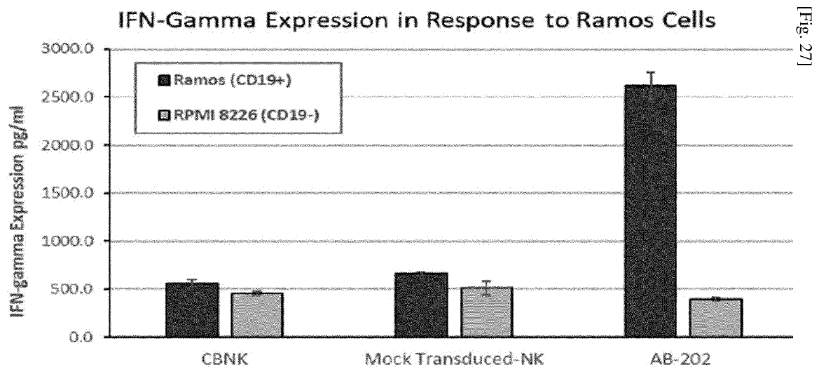

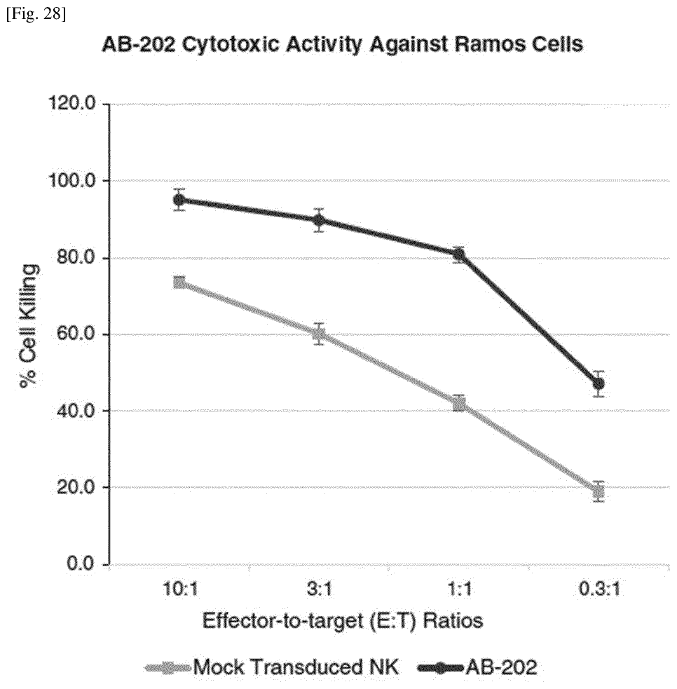

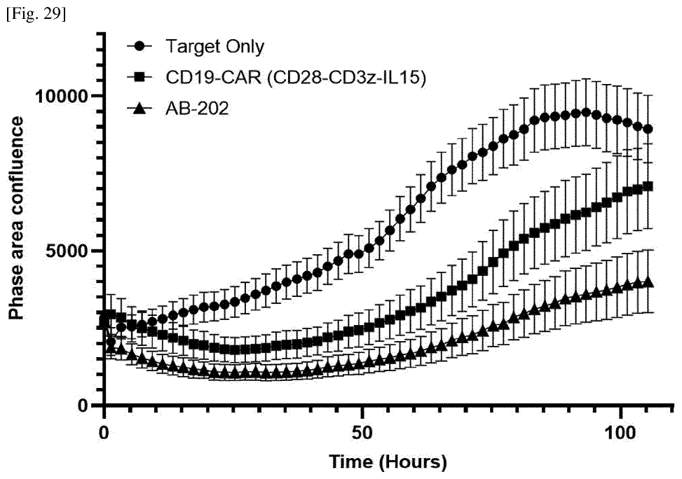

### CLAIM OF PRIORITY

This application is a 371 National Stage application no. PCT/KR2022/095077, filed on Apr. 7, 2022, which application claims the benefit of Korean Patent Application No. 10-2021-0045765, filed on Apr. 8, 2021. The entire contents of the foregoing are incorporated herein by reference.

### SEQUENCE LISTING

This application contains a Sequence Listing that has been submitted electronically as an ASCII text file named 49755-0018US1_SL_ST25.txt. The ASCII text file, created on May 14, 2024, is 94,254 bytes in size. The material in the ASCII text file is hereby incorporated by reference in its entirety.

### BACKGROUND

Targeted therapies, including the use of adoptive cell therapies such as chimeric antigen receptor T cells (CAR Ts), have revolutionized cancer treatment. These cell therapies may be autologous (CAR T cells manufactured using a patient's own T cells) or allogeneic (CAR T cells manufactured using T cells from healthy donors. Currently, there are no autologous CAR T therapies approved to treat HER2-specific cancers.

Whilst transformative in their ability to treat targeted hematologic cancers, challenging obstacles have arisen in the clinic since 2017, when the first CAR-T therapies targeting CD19 B-cell malignancies were approved by the United States Food and Drug Administration, with the use of autologous CAR-T cell products. CAR T cell manufacturing is a resource-intensive process that can result in failure to produce a viable autologous cell therapy for some patients. The average manufacturing time of 3 weeks that is needed for autologous CAR T cell products may be too long for critically ill patients. Finally, due to the complex nature of the manufacture and delivery of CAR-T cell product, which require close monitoring at top-tier cancer and medical centers, access to this treatment option may be out of reach, both financially and geographically, for most patients. Importantly, even for those patients who have access to this innovative treatment, CAR-T cell products confer a risk of serious and potentially deadly adverse effects. These adverse effects include cytokine release syndrome (CRS) and neurotoxicity, which can be difficult to manage or control.

Allogeneic CAR-T cell therapies, which utilize cells from healthy donors, may overcome some of the manufacturing and logistical challenges of autologous CAR-T cell therapies. However, these “off-the-shelf” CAR T cell therapies also have issues that include a potentially higher risk of graft-versus-host disease (GVHD) and ineffectiveness due to rapid clearance by the patient's immune system.

Similar to T cells, allogeneic NK cells engineered to express CARs with anti-tumor activity may provide an important treatment option for cancer patients. NK cells do not suffer from some of the shortcomings of allogeneic CAR-T cells, which often retain expression of endogenous T cell receptors in addition to engineered chimeric antigen receptors. As a result, allogeneic CAR-NK cell treatments can be administered safely to patients without many of the risks associated with allogeneic T cell therapies, including GVHD. However, CAR-NK cells face many of the same challenges as other allogeneic cell therapies, including product sourcing, scalability, persistence, and dose-to-dose variability. For example, the signaling and activation pathways in NK cells can differ from those of T cells, which may require different combinations of co-stimulatory molecules. In another example, NK cell therapies often require administration of cytokines to enhance the ability of those cells to persist inside of a patient. Thus, there is a need for CAR-NK cells that are engineered to enhance the persistence and cytotoxicity of NK cells.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). NK cells may have an advantage over other immune cells, such as the T cells used in CAR-T cell therapy and other cell therapies. Autologous CAR-T cells must be engineered from a patient's own cells. Such engineering can take time, during which period the patient's disease may progress significantly. Such patients may require a bridging therapy to sustain them until their autologous CAR-T cells are ready. Not all patients qualify for autologous CAR-T therapy. For example, some patients may be too sick or may not have sufficient numbers of T cells suitable for engineering purposes. Not all manufacturing runs of autologous CAR-T cells result in sufficient cell numbers or sufficiently active cells to be therapeutically effective. When such manufacturing runs are successful, patients typically only receive a single dose of autologous CAR-T treatment. Because the risk of acute side effects like ICANS and CRS are greatest immediately after administering CAR-T cells, repeat dosing is potentially too risky if the patient will only see marginal benefit from a second, third, or further dose. Additionally, because autologous CAR-T treatments must be unique for each patient, the costs of such treatments can make them unaffordable for many patients who would otherwise benefit from them.

In an exemplary advantage, NK cells can be used as allogeneic therapies, meaning that NK cells from one donor can be safely used in one or many patients without the requirement for HLA matching, gene editing, or other genetic manipulations. As a result, allogeneic CAR-NK cells can be manufactured in bulk, cryopreserved, shipped throughout the world, and administered on demand at the point of care. Thus, the allogeneic cell therapies can be administered to a patient immediately, without the need to wait for the patient's own cells to be engineered and administered and without the need for a bridging therapy. Because the allogeneic therapies described herein can be manufactured in bulk using campaign-manufacturing methods, the costs associated with manufacturing and delivering the allogeneic therapies described herein has the promise to be significantly lower than those of autologous CAR-T therapies. Campaign manufacturing also reduces variability between batches and allows a patient to receive multiple doses of CAR-NK cells made from a single batch derived from a single donor where preferable.

The ability to offer repeat dosing may allow patients to experience or maintain a deeper or prolonged response from the therapy. For example, patients can receive response-based dosing, during which the patient continues to receive doses of CAR-NK cell therapy for as long as the patient derives a benefit. The number of doses and the number of cells administered in each dose can also be tailored to the individual patient. In such cases, the patient is not limited by the number of cells he or she can provide during the cell harvests associated with autologous CAR-T therapy. Thus, the CAR-NK cell therapies described herein can be tailored to each patient based on that patient's own response. In some cases, the therapy can also be reinitiated if the patient relapses.

Allogeneic NK cells may provide an important treatment option for cancer patients. In one exemplary advantage, NK cells have been well tolerated without evidence of graft-versus-host disease, neurotoxicity or cytokine release syndrome associated with other cell-based therapies. In another exemplary advantage, NK cells do not require prior antigen exposure to antigens to identify and lyse tumor cells. In another exemplary advantage, NK cells have the inherent ability to bridge between innate immunity and engender a multi-clonal adaptive immune response resulting in long-term anticancer immune memory. All of these features contribute to the potential for NK cell efficacy as cancer treatment options.

For example, NK cells can recruit and activate other components of the immune system. Activated NK cells secrete cytokines and chemokines, such as interferon gamma (IFNγ); tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system.

Additionally, umbilical cord blood units with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

Engineered NK cells, e.g., the CAR-NK cells described herein, have an advantage over autologous cell therapies, e.g., T cells used in CAR-T cell therapy, because the NK cells can be used as allogeneic therapies. Thus, NK cells from one donor can be safely used in one or many patients.

Provided herein, amongst other things, are polynucleotides comprising a) a chimeric antigen receptor (CAR) comprising: an extracellular antigen binding domain comprising an antibody or antigen binding fragment thereof; a transmembrane region; and an intracellular signaling region comprising an OX40L intracellular signaling domain; and b) a nucleic acid encoding an IL-15.

Provided herein are polynucleotide(s) comprising: a) a nucleic acid encoding a chimeric antigen receptor (CAR) comprising an intracellular signaling region comprising an OX40L intracellular signaling domain; and b) a nucleic acid encoding an IL-15.

In some embodiments, the intracellular signaling region comprises a CD28 intracellular signaling domain and a CD3-zeta signaling domain.

In some embodiments, the OX40L intracellular signaling domain comprises an amino acid sequence set forth in SEQ ID NO: 8, SEQ ID NO: 9, or SEQ ID NO: 10.

In some embodiments, the OX40L intracellular signaling domain comprises an amino acid sequence having at least 85%, 86%, 87%, 88%, 89%, 90%, 91%, 92%, 93%, 94%, 95%, 96%, 97%, 98%, or 99% identity to SEQ ID NO: 8, SEQ ID NO: 9, or SEQ ID NO: 10.

In some embodiments, the OX40L intracellular signaling domain is encoded by a nucleic acid comprising SEQ ID NO: 11 or SEQ ID NO: 12.

In some embodiments, the intracellular signaling region further comprises a CD28 intracellular signaling domain.

In some embodiments, the CD28 intracellular signaling domain comprises an amino acid sequence set forth in SEQ ID NO: 5.

In some embodiments, the CD28 intracellular signaling domain comprises an amino acid sequence having at least 85%, 86%, 87%, 88%, 89%, 90%, 91%, 92%, 93%, 94%, 95%, 96%, 97%, 98%, or 99% identity to SEQ ID NO: 5.

In some embodiments, the CD28 intracellular signaling domain is encoded by a nucleic acid comprising SEQ ID NO: 6 or SEQ ID NO: 7.

In some embodiments, the intracellular signaling region further comprises an CD3-zeta intracellular signaling domain.

In some embodiments, the CD3-zeta intracellular signaling domain comprises an amino acid sequence set forth in SEQ ID NO: 13.

In some embodiments, the CD3-zeta intracellular signaling domain comprises an amino acid sequence having at least 85%, 86%, 87%, 88%, 89%, 90%, 91%, 92%, 93%, 94%, 95%, 96%, 97%, 98%, or 99% identity to SEQ ID NO: 13.

In some embodiments, the CD3-zeta intracellular signaling domain is encoded by a nucleic acid comprising SEQ ID NO: 14 or SEQ ID NO: 15.

In some embodiments, the IL-15 comprises the amino acid sequence set forth in SEQ ID NO: 22.

In some embodiments, the IL-15 comprises an amino acid sequence having at least 85%, 86%, 87%, 88%, 89%, 90%, 91%, 92%, 93%, 94%, 95%, 96%, 97%, 98%, or 99% identity to SEQ ID NO: 22.

In some embodiments, the IL-15 is encoded by a nucleic acid comprising SEQ ID NO: 17 or SEQ ID NO: 18.

In some embodiments, the polynucleotide encodes a polyprotein comprising the CAR and the IL-15.

In some embodiments, the polynucleotide further comprises a nucleic acid encoding a self-cleaving peptide.

In some embodiments, the CAR is joined to the IL-15 by the self-cleaving peptide.

In some embodiments, the self-cleaving peptide is a T2A self-cleaving peptide.

In some embodiments, the T2A self-cleaving peptide comprises an amino acid sequence set forth in SEQ ID NO: 14 or SEQ ID NO: 18.

In some embodiments, the T2A self-cleaving peptide is capable of inducing ribosomal skipping between the CAR and the IL-15.

In some embodiments, the chimeric antigen receptor further comprises a transmembrane domain.

In some embodiments, the transmembrane domain is a CD28 transmembrane domain.

In some embodiments, the transmembrane domain comprises an amino acid sequence set forth in SEQ ID NO: 32.

In some embodiments, the transmembrane domain comprises an amino acid sequence having at least 85%, 86%, 87%, 88%, 89%, 90%, 91%, 92%, 93%, 94%, 95%, 96%, 97%, 98%, or 99% identity to SEQ ID NO: 32.

In some embodiments, the transmembrane domain is encoded by a nucleic acid comprising SEQ ID NO: 33 or SEQ ID NO: 34.

In some embodiments, the chimeric antigen receptor further comprises a hinge domain.

In some embodiments, the hinge domain is a CD8a hinge domain.

In some embodiments, the hinge domain comprises an amino acid sequence set forth in SEQ ID NO: 29.

In some embodiments, the hinge domain comprises an amino acid sequence having at least 85%, 86%, 87%, 88%, 89%, 90%, 91%, 92%, 93%, 94%, 95%, 96%, 97%, 98%, or 99% identity to SEQ ID NO: 29.

In some embodiments, the hinge domain is encoded by a nucleic acid comprising SEQ ID NO: 30 or SEQ ID NO: 31.

In some embodiments, the chimeric antigen receptor further comprises an antigen binding domain.

In some embodiments, the antigen binding domain comprises an antigen binding fragment.

In some embodiments, the antigen binding fragment is a single chain Fv (scFv).

In some embodiments, the antigen binding domain comprises a V_{L} region and a V_{H} region.

In some embodiments, the V_{L} region is amino terminal to the V_{H} region.

In some embodiments, the V_{L} region is carbosy terminal to the V_{H} region.

Also provided herein are vector(s) comprising the polynucleotide(s) described herein.

In some embodiments, the vector is a viral vector.

In some embodiments, the viral vector is a retroviral vector or a lentiviral vector.

Also provided herein are cell(s) comprising the polynucleotide(s) and/or vector(s) described herein.

Also provided herein are cell(s) expressing the chimeric antigen receptor(s) and the IL-15 encoded by the polynucleotide(s) and/or vector(s) described herein.

In some embodiments, the cell is a lymphocyte.

In some embodiments, the lymphocyte is a natural killer (NK) cell.

In some embodiments, the lymphocyte is a T cell.

In some embodiments, the cell is a human cell.

In some embodiments, the cell is a primary cell obtained from a subject.

In some embodiments, the cell is a primary cell obtained from cord blood.

In some embodiments, the cell comprises a KIR-B haplotype.

In some embodiments, the cell express CD16 having the V/V polymorphism at F158.

Also provided herein are population(s) of cells comprising a plurality of the cells described herein.

In some embodiments, at least 10%, 20%, 30%, 40%, 50%, 60%, 70%, 80%, 85%, 90%; 95%, 96%, 97%, 98%, or 99% of the cells comprise the polynucleotide(s) and/or vector(s) described herein.

In some embodiments, at least 10%, 20%, 30%, 40%, 50%, 60%, 70%, 80%, 85%, 90%; 95%, 96%, 97%, 98%, or 99% of the cells express the chimeric antigen receptor and the IL-15 encoded by the polynucleotide(s) and/or vector(s) described herein.

Also provided herein are pharmaceutical composition(s) comprising the population(s) of cells described herein.

In some embodiments, the pharmaceutical composition further comprises a pharmaceutically acceptable excipient.

In some embodiments, the pharmaceutical composition further comprises: (a) human albumin; (b) dextran; (c) glucose; (d) DMSO; and (e) a buffer.

In some embodiments, the pharmaceutical composition comprises from 30 to 50 mg/mL human albumin.

In some embodiments, the pharmaceutical composition comprises 50 mg/mL human albumin.

In some embodiments, the pharmaceutical composition comprises 20 to 30 mg/mL dextran.

In some embodiments, the pharmaceutical composition comprises 25 mg/mL dextran.

In some embodiments, the dextran is Dextran 40.

In some embodiments, the pharmaceutical composition comprises from 12 to 15 mg/mL glucose.

In some embodiments, the pharmaceutical composition comprises 12.5 mg/mL glucose.

In some embodiments, the pharmaceutical composition comprises less than 27.5 g/L glucose.

In some embodiments, the pharmaceutical composition comprises from 50 to 60 ml/mL DMSO.

In some embodiments, the pharmaceutical composition comprises 55 mg/mL DMSO.

In some embodiments, the pharmaceutical composition comprises 40 to 60% v/v buffer.

In some embodiments, the buffer is phosphate buffered saline.

In some embodiments, the pharmaceutical composition further comprises water.

In some embodiments, the pharmaceutical composition comprises: (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline.

In some embodiments, the pharmaceutical composition further comprises 0.5 mL/mL water.

Also provided herein are frozen vial(s) comprising the composition(s) described herein.

Also provided herein are methods of treatment comprising administering the cell(s), population(s) of cells, and/or composition(s) described herein to a subject having a disease or condition.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGURES, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

Error! Reference source not found. shows that CAR-NKs comprising a co-stimulatory domain comprising OX40L exhibited greater cytotoxic potential than those without OX40L.

Error! Reference source not found. shows schematics of CAR constructs.

Error! Reference source not found. shows proliferation of the CAR constructs of Error! Reference source not found.

Error! Reference source not found. shows CAR expression of the CAR constructs of Error! Reference source not found.

Error! Reference source not found. shows CD107a expression of the CAR constructs of Error! Reference source not found. Bars, from left to right: Mock, 2^{nd}-CAR, 3^{rd}-CAR.

Error! Reference source not found. shows IFN-γ expression of the CAR constructs of Error! Reference source not found. Bars, from left to right: Mock, 2^{nd}-CAR, 3^{rd}-CAR.

Error! Reference source not found. shows TNF-α expression of the CAR constructs of Error! Reference source not found. Bars, from left to right: Mock, 2^{nd}-CAR, 3^{rd}-CAR.

Error! Reference source not found. shows short term cytotoxicity of the CAR constructs of Error! Reference source not found.

Error! Reference source not found. shows schemes of CAR constructs. From top to bottom: mock GFP expressing NK (mock-NK); CAR without IL-15 (CAR-NK); truncated CAR with IL-15 (CAR (t)-IL-15-NK); CAR with IL-15 (CAR-IL-15-NK, AB-201).

Error! Reference source not found. shows CAR expression on NK cells cultured in the presence of IL-2.

Error! Reference source not found. shows CAR expression on NK cells cultured in the absence of IL-2.

Error! Reference source not found. shows proliferation of NK cells cultured in the presence of IL-2 and in the absence of IL-2.

Error! Reference source not found. shows viability of NK cells cultured in the presence of IL-2 and in the absence of IL-2.

Error! Reference source not found. shows cytotoxicity of NK cells.

Error! Reference source not found. shows IFNg production of NK cells.

Error! Reference source not found. shows IL-15 production of NK cells.

Error! Reference source not found. demonstrates that secretion of IL-15 Maintains the Survival of Bystander NK Cells.

Error! Reference source not found. is a schematic of two different CAR structures.

Error! Reference source not found. shows CAR expression of the CAR structures shown in Error! Reference source not found. over time (in days).

Error! Reference source not found. shows survival of cells expressing the CAR structures shown in Error! Reference source not found. (cell numbers).

Error! Reference source not found. shows survival of cells expressing the CAR structures shown in Error! Reference source not found. (percent viability).

Error! Reference source not found. shows the number of viable NK cells after 7 days of co-culture of cells expressing the CAR structures shown in Error! Reference source not found. with target cells without IL-2 support. Bars, from left to right: cord blood NK cells (CBNK); 3rd CAR-NK; 4th CAR-NK.

Error! Reference source not found. is a schematic of two different CAR structures.

Error! Reference source not found. shows short term cytotoxicity of NK cells expressing the CAR structures shown in Error! Reference source not found. against Raji (CD19+) cells).

Error! Reference source not found. shows short term cytotoxicity of NK cells expressing the CAR structures shown in Error! Reference source not found. against Ramos (CD19+) cells).

Error! Reference source not found. shows short term cytotoxicity of NK cells expressing the CAR structures shown in Error! Reference source not found. against RPMI8226 (CD19−) cells).

Error! Reference source not found. shows AB-202 in Vitro Cytotoxicity.

Error! Reference source not found. shows AB-202 in Vitro Cytotoxicity.

Error! Reference source not found. shows AB-202 in Vitro Cytotoxicity.

### DETAILED DESCRIPTION

Provided herein are, amongst other things, Natural Killer (NK) cells, e.g., CAR-NK cells, methods for producing the NK cells, pharmaceutical compositions comprising the NK cells, and methods of treating patients suffering, e.g., from cancer, with the NK cells.

### I. EXPANSION AND STIMULATION OF NATURAL KILLER CELLS

In some embodiments, natural killer cells are expanded and stimulated, e.g., by culturing and stimulation with feeder cells.

NK cells can be expanded and stimulated as described, for example, in US 2020/0108096 or WO 2020/101361, both of which are incorporated herein by reference in their entirety. Briefly, the source cells can be cultured on modified HuT-78 (ATCC® TIB-161™) cells that have been engineered to express 4-1BBL, membrane bound IL-21, and a mutant TNFα as described in US 2020/0108096.

Suitable NK cells can also be expanded and stimulated as described herein.

In some embodiments, NK cells are expanded and stimulated by a method comprising: (a) providing NK cells, e.g., a composition comprising NK cells, e.g., CD3(−) depleted cells; and (b) culturing in a medium comprising feeder cells and/or stimulation factors, thereby producing a population of expanded and stimulated NK cells.

### A. Natural Killer Cell Sources

In some embodiments, the NK cell source is selected from the group consisting of peripheral blood, peripheral blood lymphocytes (PBLs), peripheral blood mononuclear cells (PBMCs), bone marrow, umbilical cord blood (cord blood), isolated NK cells, NK cells derived from induced pluripotent stem cells, NK cells derived from embryonic stem cells, and combinations thereof.

In some embodiments, the NK cell source is a single unit of cord blood.

In some embodiments, the natural killer cell source, e.g., single unit of cord blood, comprises from or from about 1×10⁷ to or to about 1×10⁹ total nucleated cells. In some embodiments, the natural killer cell source, e.g., single unit of cord blood, comprises from or from about 1×10⁸ to or to about 1.5×10⁸ total nucleated cells. In some embodiments, the natural killer cell source, e.g., single unit of cord blood, comprises 1×10⁸ total nucleated cells. In some embodiments, the natural killer cell source, e.g., single unit of cord blood, comprises about 1×10⁸ total nucleated cells. In some embodiments, the natural killer cell source, e.g., single unit of cord blood, comprises 1×10⁹ total nucleated cells. In some embodiments, the natural killer cell source, e.g., single unit of cord blood, comprises about 1×10⁹ total nucleated cells.

In some embodiments, the NK cell source, e.g., the cord blood unit, comprises from about 20% to about 80% CD16+ cells. In some embodiments, the NK cell source, e.g., the cord blood unit, comprises from or from about 20% to or to about 80%, from about 20% to or to about 70%, from about 20% to or to about 60%, from about 20% to or to about 50%, from about 20% to or to about 40%, from about 20% to or to about 30%, from about 30% to or to about 80%, from about 30% to or to about 70%, from about 30% to or to about 60%, from about 30% to or to about 50%, from about 30% to or to about 40%, from about 40% to or to about 80%, from about 40% to or to about 70%, from about 40% to or to about 60%, from about 40% to or to about 50%, from about 50% to or to about 80%, from about 50% to or to about 70%, from about 50% to or to about 60%, from about 60% to or to about 80%, from about 60% to or to about 70%, or from about 70% to or to about 80% CD16+ cells. In some embodiments, the NK cell source, e.g., the cord blood unit, comprises less than 80% CD16+ cells. Alternately, some NK cell sources may comprise CD16+ cells at a concentration of greater than 80%

In some embodiments, the NK cell source, e.g., the cord blood unit, comprises less than 40%, e.g., less than 30%, e.g., less than 20%, e.g., less than 10%, e.g., less than 5% MLG2A+ cells.

In some embodiments, the NK cell source, e.g., the cord blood unit, comprises less than 40%, e.g., less than 30%, e.g., less than 20%, e.g., less than 10%, e.g., less than 5% NKG2C+ cells.

In some embodiments, the NK cell source, e.g., the cord blood unit, comprises less than 40%, e.g., less than 30%, e.g., less than 20%, e.g., less than 10%, e.g., less than 5% NKG2D+ cells.

In some embodiments, the NK cell source, e.g., the cord blood unit, comprises less than 40%, e.g., less than 30%, e.g., less than 20%, e.g., less than 10%, e.g., less than 5% NKp46+ cells.

In some embodiments, the NK cell source, e.g., the cord blood unit, comprises less than 40%, e.g., less than 30%, e.g., less than 20%, e.g., less than 10%, e.g., less than 5% NKp30+ cells.

In some embodiments, the NK cell source, e.g., the cord blood unit, comprises less than 40%, e.g., less than 30%, e.g., less than 20%, e.g., less than 10%, e.g., less than 5% DNAM-1+ cells.

In some embodiments, the NK cell source, e.g., the cord blood unit, comprises less than 40%, e.g., less than 30%, e.g., less than 20%, e.g., less than 10%, e.g., less than 5% NKp44+ cells.

In some embodiments, the NK cell source, e.g., the cord blood unit, comprises less than 40%, e.g., less than 30%, e.g., less than 20%, e.g., less than 10%, e.g., less than 5% CD25+ cells.

In some embodiments, the NK cell source, e.g., the cord blood unit, comprises less than 40%, e.g., less than 30%, e.g., less than 20%, e.g., less than 10%, e.g., less than 5% CD62L+ cells.

In some embodiments, the NK cell source, e.g., the cord blood unit, comprises less than 40%, e.g., less than 30%, e.g., less than 20%, e.g., less than 10%, e.g., less than 5% CD69+ cells.

In some embodiments, the NK cell source, e.g., the cord blood unit, comprises less than 40%, e.g., less than 30%, e.g., less than 20%, e.g., less than 10%, e.g., less than 5% CXCR3+ cells.

In some embodiments, the NK cell source, e.g., the cord blood unit, comprises less than 40%, e.g., less than 30%, e.g., less than 20%, e.g., less than 10%, e.g., less than 5% CD57+ cells.

In some embodiments, NK cells in the NK cell source comprise a KIR B allele of the KIR receptor family. See, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5: e15115 (2010).

In some embodiments, NK cells in the NK cell source comprise the 158 V/V variant of CD16 (i.e. homozygous CD16 15V polymorphism). See, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).

In some embodiments, NK cells in the cell source comprises both the KIR B allele of the KIR receptor family and the 158 V/V variant of CD16.

In some embodiments, the NK cells in the cell source are not genetically engineered.

In some embodiments, the NK cells in the cell source do not comprise a CD16 transgene.

In some embodiments, the NK cells in the cell source do not express an exogenous CD16 protein.

In some embodiments, the NK cell source is CD3(+) depleted. In some embodiments, the method comprises depleting the NK cell source of CD3(+) cells. In some embodiments, depleting the NK cell source of CD3(+) cells comprises contacting the NK cell source with a CD3 binding antibody or antigen binding fragment thereof. In some embodiments, the CD3 binding antibody or antigen binding fragment thereof is selected from the group consisting of OKT3, UCHT1, and HIT3a, and fragments thereof. In some embodiments, the CD3 binding antibody or antigen binding fragment thereof is OKT3 or an antigen binding fragment thereof. In some embodiments, the antibody or antigen binding fragment thereof is attached to a bead, e.g., a magnetic bead. In some embodiments, the depleting the composition of CD3(+) cells comprises contacting the composition with a CD3 targeting antibody or antigen binding fragment thereof attached to a bead and removing the bead-bound CD3(+) cells from the composition. The composition can be depleted of CD3 cells by immunomagnetic selection, for example, using a CliniMACS T cell depletion set ((LS Depletion set (162-01) Miltenyi Biotec).

In some embodiments, the NK cell source CD56+ enriched, e.g., by gating on CD56 expression.

In some embodiments, the NK cell source is both CD56+ enriched and CD3(+) depleted, e.g., by selecting for cells with CD56+CD3− expression.

In some embodiments, the NK cell source comprises both the KIR B allele of the KIR receptor family and the 158 V/V variant of CD16 and is + enriched and CD3(+) depleted, e.g., by selecting for cells with CD56+CD3− expression.

### B. Feeder Cells

Disclosed herein are feeder cells for the expansion of NK cells. These feeder cells advantageously allow NK cells to expand to numbers suitable for the preparation of a pharmaceutical composition as discussed herein. In some cases, the feeder cells allow the expansion of NK cells without the loss of CD16 expression, which often accompanies cell expansion on other types of feeder cells or using other methods. In some cases, the feeder cells make the expanded NK cells more permissive to freezing such that a higher proportion of NK cells remain viable after a freeze/thaw cycle or such that the cells remain viable for longer periods of time while frozen. In some cases, the feeder cells allow the NK cells to retain high levels of cytotoxicity, including ADCC, extend survival, increase persistence, and enhance or retain high levels of CD16. In some cases, the feeder cells allow the NK cells to expand without causing significant levels of exhaustion or senescence.

Feeder cells can be used to stimulate the NK cells and help them to expand more quickly, e.g., by providing substrate, growth factors, and/or cytokines.

NK cells can be stimulated using various types of feeder cells, including, but not limited to peripheral blood mononuclear cells (PBMC), Epstein-Barr virus-transformed B-lymphoblastoid cells (e.g., EBV-LCL), myelogenous leukemia cells (e.g., K562), and CD4(+) T cells (e.g., HuT), and derivatives thereof.

In some embodiments, the feeder cells are inactivated, e.g., by γ-irradiation or mitomycin-c treatment.

Suitable feeder cells for use in the methods described herein are described, for example, in US 2020/0108096, which is hereby incorporated by reference in its entirety.

In some embodiments, the feeder cell(s) are inactivated CD4(+) T cell(s). In some embodiments, the inactivated CD4(+) T cell(s) are HuT-78 cells (ATCC® TIB-161™) or variants or derivatives thereof. In some embodiments, the HuT-78 derivative is H9 (ATCC® HTB-176™).

In some embodiments, the inactivated CD4(+) T cell(s) express OX40L. In some embodiments, the inactivated CD4(+) T cell(s) are HuT-78 cells or variants or derivatives thereof that express OX40L (SEQ ID NO: 4) or a variant thereof.

In some embodiments, the feeder cells are HuT-78 cells engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

In some embodiments, the inactivated CD4(+) T cell(s) are HuT-78 (ATCC® TIB-161™) cells or variants or derivatives thereof that express an ortholog of OX40L, or variant thereof. In some embodiments, the feeder cells are HuT-78 cells engineered to express at least one gene selected from the group consisting of an 4-1BBL ortholog or variant thereof, a membrane bound IL-21 ortholog or variant thereof, and mutant TNFalpha ortholog, or variant thereof.

In some embodiments, the feeder cells are HuT-78 cell(s) that express OX40L (SEQ ID NO: 4) and are engineered to express 4-1BBL (SEQ ID NO: 1), membrane bound IL-21 (SEQ ID NO: 2), and mutant TNFalpha (SEQ ID NO: 3) (“eHut-78 cells”) or variants or derivatives thereof.

In some embodiments, the feeder cells are expanded, e.g., from a frozen stock, before culturing with NK cells.

### C. Stimulating Factors

NK cells can also be stimulated using one or more stimulation factors other than feeder cells, e.g., signaling factors, in addition to or in place of feeder cells.

In some embodiments, the stimulating factor, e.g., signaling factor, is a component of the culture medium, as described herein. In some embodiments, the stimulating factor, e.g., signaling factor, is a supplement to the culture medium, as described herein.

In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-2.

In some embodiments, the cytokines are a combination of IL-2 and IL-15.

In some embodiments, the cytokines are a combination of IL-2, IL-15, and IL-18.

In some embodiments, the cytokines are a combination of IL-2, IL-18, and IL-21.

### D. Culturing

The NK cells can be expanded and stimulated by co-culturing an NK cell source and feeder cells and/or other stimulation factors. Suitable NK cell sources, feeder cells, and stimulation factors are described herein.

In some cases, the resulting population of expanded natural killer cells is enriched and/or sorted after expansion. In some cases, the resulting population of expanded natural killer cells is not enriched and/or sorted after expansion

Also described herein are compositions comprising the various culture compositions described herein, e.g., comprising NK cells. For example, a composition comprising a population of expanded cord blood-derived natural killer cells comprising a KIR-B haplotype and homozygous for a CD16 158V polymorphism and a plurality of engineered HuT78 cells.

Also described herein are vessels, e.g., vials, cryobags, and the like, comprising the resulting populations of expanded natural killer cells. In some cases, a plurality of vessels comprising portions of the resulting populations of expanded natural killer cells, e.g., at least 10, e.g., 20, 30, 40, 50, 60, 70, 80, 90, 100, 125, 150, 175, 200, 250, 300, 400, 500, 600, 700, 800, 900, 1000, 1100, or 1200 vessels.

Also described herein are bioreactors comprising the various culture compositions described herein, e.g., comprising NK cells. For example, a culture comprising natural killer cells from a natural killer cell source, e.g., as described herein, and feeder cells, e.g., as described herein. Also described herein are bioreactors comprising the resulting populations of expanded natural killer cells.

### 1. Culture Medium

Disclosed herein are culture media for the expansion of NK cells. These culture media advantageously allow NK cells to expand to numbers suitable for the preparation of a pharmaceutical composition as discussed herein. In some cases, the culture media allows NK cells to expand without the loss of CD16 expression that often accompanies cell expansion on other helper cells or in other media.

In some embodiments, the culture medium is a basal culture medium, optionally supplemented with additional components, e.g., as described herein.

In some embodiments, the culture medium, e.g., the basal culture medium, is a serum-free culture medium. In some embodiments, the culture medium, e.g., the basal culture medium, is a serum-free culture medium supplemented with human plasma and/or serum.

Suitable basal culture media include, but are not limited to, DMEM, RPMI 1640, MEM, DMEM/F12, SCGM (CellGenix®, 20802-0500 or 20806-0500), LGM-3™ (Lonza, CC-3211), TexMACS™ (Miltenyi Biotec, 130-097-196), ALyS™ 505NK-AC (Cell Science and Technology Institute, Inc., 01600P02), ALyS™ 505NK-EX (Cell Science and Technology Institute, Inc., 01400P10), CTS™ AIM-V™ SFM (ThermoFisher Scientific, A3830801), CTS™ OpTmizer™ (ThermoFisher Scientific, A1048501, ABS-001, StemXxVivoand combinations thereof.

The culture medium may comprise additional components, or be supplemented with additional components, such as growth factors, signaling factors, nutrients, antigen binders, and the like. Supplementation of the culture medium may occur by adding each of the additional component or components to the culture vessel either before, concurrently with, or after the medium is added to the culture vessel. The additional component or components may be added together or separately. When added separately, the additional components need not be added at the same time.

In some embodiments, the culture medium comprises plasma, e.g., human plasma. In some embodiments, the culture medium is supplemented with plasma, e.g., human plasma. In some embodiments, the plasma, e.g., human plasma, comprises an anticoagulant, e.g., trisodium citrate.

In some embodiments, the medium comprises and/or is supplemented with from or from about 0.5% to or to about 10% v/v plasma, e.g., human plasma. In some embodiments, the medium is supplemented with from or from about 0.5% to or to about 9%, from or from about 0.5% to or to about 8%, from or from about 0.5% to or to about 7%, from or from about 0.5% to or to about 6%, from or from about 0.5% to or to about 5%, from or from about 0.5% to or to about 4%, from or from about 0.5% to or to about 3%, from or from about 0.5% to or to about 2%, from or from about 0.5% to or to about 1%, from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 2% to or to about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 4% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v plasma, e.g., human plasma. In some embodiments, the culture medium comprises and/or is supplemented with from 0.8% to 1.2% v/v human plasma. In some embodiments, the culture medium comprises and/or is supplemented with 1.0% v/v human plasma. In some embodiments, the culture medium comprises and/or is supplemented with about 1.0% v/v human plasma.

In some embodiments, the culture medium comprises serum, e.g., human serum. In some embodiments, the culture medium is supplemented with serum, e.g., human serum. In some embodiments, the serum is inactivated, e.g., heat inactivated. In some embodiments, the serum is filtered, e.g., sterile-filtered.

In some embodiments, the culture medium comprises glutamine. In some embodiments, the culture medium is supplemented with glutamine. In some embodiments, the culture medium comprises and/or is supplemented with from or from about 2.0 to or to about 6.0 mM glutamine. In some embodiments, the culture medium comprises and/or is supplemented with from or from about 2.0 to or to about 5.5, from or from about 2.0 to or to about 5.0, from or from about 2.0 to or to about 4.5, from or from about 2.0 to or to about 4.0, from or from about 2.0 to or to about 3.5, from or from about 2.0 to or to about 3.0, from or from about 2.0 to or to about 2.5, from or from about 2.5 to or to about 6.0, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3.0 to or to about 6.0, from or from about 3.0 to or to about 5.5, from or from about 3.0 to or to about 5.0, from or from about 3.0 to or to about 4.5, from or from about 3.0 to or to about 4.0, from or from about 3.0 to or to about 3.5, from or from about 3.5 to or to about 6.0, from or from about 3.5 to or to about 5.5, from or from about 3.5 to or to about 5.0, from or from about 3.5 to or to about 4.5, from or from about 3.5 to or to about 4.0, from or from about 4.0 to or to about 6.0, from or from about 4.0 to or to about 5.5, from or from about 4.0 to or to about 5.0, from or from about 4.0 to or to about 4.5, from or from about 4.5 to or to about 6.0, from or from about 4.5 to or to about 5.5, from or from about 4.5 to or to about 5.0, from or from about 5.0 to or to about 6.0, from or from about 5.0 to or to about 5.5, or from or from about 5.5 to or to about 6.0 mM glutamine. In some embodiments, the culture medium comprises and/or is supplemented with from 3.2 mM glutamine to 4.8 mM glutamine. In some embodiments, the culture medium comprises and/or is supplemented with 4.0 mM glutamine. In some embodiments, the culture medium comprises and/or is supplemented with about 4.0 mM glutamine.

In some embodiments, the culture medium comprises one or more cyotkines. In some embodiments, the culture medium is supplemented with one or more cyotkines.

In some embodiments, the cytokine is selected from IL-2, IL-12, IL-15, IL-18, and combinations thereof.

In some embodiments, the culture medium comprises and/or is supplemented with IL-2. In some embodiments, the culture medium comprises and/or is supplemented with from or from about 150 to or to about 2,500 IU/mL IL-2. In some embodiments, the culture medium comprises and/or is supplemented with from or from about 200 to or to about 2,250, from or from about 200 to or to about 2,000, from or from about 200 to or to about 1,750, from or from about 200 to or to about 1,500, from or from about 200 to or to about 1,250, from or from 200 to or to about 1,000, from or from about 200 to or to about 750, from or from about 200 to or to about 500, from or from about 200 to or to about 250, from or from about 250 to or to about 2,500, from or from about 250 to or to about 2,250, from or from about 250 to or to about 2,000, from or from about 250 to or to about 1,750, from or from about 250 to or to about 1,500, from or from about 250 to or to about 1,250, from or from about 250 to or to about 1,000, from or from about 250 to or to about 750, from or from about 250 to or to about 500, from or from about 500 to or to about 2,500, from or from about 500 to or to about 2,250, from or from about 500 to or to about 2,000, from or from about 500 to or to about 1,750, from or from about 500 to or to about 1,500, from or from about 500 to or to about 1,250, from or from about 500 to or to about 1,000, from or from about 500 to or to about 750, from or from about 750 to or to about 2,250, from or from about 750 to or to about 2,000, from or from about 750 to or to about 1,750, from or from about 750 to or to about 1,500, from or from about 750 to or to about 1,250, from or from about 750 to or to about 1,000, from or from about 1,000 to or to about 2,500, from or from about 1,000 to or to about 2,250, from or from about 1,000 to or to about 2,000, from or from about 1,000 to or to about 1,750, from or from about 1,000 to or to about 1,500, from or from about 1,000 to or to about 1,250, from or from about 1,250 to or to about 2,500, from or from about 1,250 to or to about 2,250, from or from about 1,250 to or to about 2,000, from or from about 1,250 to or to about 1,750, from or from about 1,250 to or to about 1,500, from or from about 1,500 to or to about 2,500, from or from about 1,500 to or to about 2,250, from or from about 1,500 to or to about 2,000, from or from about 1,500 to or to about 1,750, from or from about 1,750 to or to about 2,500, from or from about 1,750 to or to about 2,250, from or from about 1,750 to or to about 2,000, from or from about 2,000 to or to about 2,500, from or from about 2,000 to or to about 2,250, or from or from about 2,250 to or to about 2,500 IU/mL IL-2.

In some embodiments, the culture medium comprises and/or is supplemented with from 64 μg/L to 96 μg/L IL-2. In some embodiments, the culture medium comprises and/or is supplemented with 80 μg/L IL-2 (approximately 1,333 IU/mL). In some embodiments, the culture medium comprises and/or is supplemented with about 80 μg/L.

In some embodiments, the culture medium comprises and/or is supplemented with a combination of IL-2 and IL-15.

In some embodiments, the culture medium comprises and/or is supplemented with a combination of IL-2, IL-15, and IL-18.

In some embodiments, the culture medium comprises and/or is supplemented with a combination of IL-2, IL-18, and IL-21.

In some embodiments, the culture medium comprises and/or is supplemented with glucose. In some embodiments, the culture medium comprises and/or is supplemented with from or from about 0.5 to or to about 3.5 g/L glucose. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L glucose. In some embodiments, the culture medium comprises and/or is supplemented with from 1.6 to 2.4 g/L glucose. In some embodiments, the culture medium comprises and/or is supplemented with 2.0 g/L glucose. In some embodiments, the culture medium comprises about 2.0 g/L glucose.

In some embodiments, the culture medium comprises and/or is supplemented with sodium pyruvate. In some embodiments, the culture medium comprises and/or is supplemented with from or from about 0.1 to or to about 2.0 mM sodium pyruvate. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 mM sodium pyruvate. In some embodiments, the culture medium comprises from 0.8 to 1.2 mM sodium pyruvate. In some embodiments, the culture medium comprises 1.0 mM sodium pyruvate. In some embodiments, the culture medium comprises about 1.0 mM sodium pyuruvate.

In some embodiments, the culture medium comprises and/or is supplemented with sodium hydrogen carbonate. In some embodiments, the culture medium comprises and/or is supplemented with from or from about 0.5 to or to about 3.5 g/L sodium hydrogen carbonate. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L sodium hydrogen carbonate. In some embodiments, the culture medium comprises and/or is supplemented with from 1.6 to 2.4 g/L sodium hydrogen carbonate. In some embodiments, the culture medium comprises and/or is supplemented with 2.0 g/L sodium hydrogen carbonate. In some embodiments, the culture medium comprises about 2.0 g/L sodium hydrogen carbonate.

In some embodiments, the culture medium comprises and/or is supplemented with albumin, e.g., human albumin, e.g., a human albumin solution described herein. In some embodiments, the culture medium comprises and/or is supplemented with from or from about 0.5% to or to about 3.5% v/v of a 20% albumin solution, e.g., a 20% human albumin solution. In some embodiments, the culture medium comprises and/or is supplemented with from or from about 0.5% to or to about 3.0%, from or from about 0.5% to or to about 2.5%, from or from about 0.5% to or to about 2.0%, from or from about 0.5% to or to about 1.5%, from or from about 0.5% to or to about 1.0%, from or from about 1.0% to or to about 3.0%, from or from about 1.0% to or to about 2.5%, from or from about 1.0% to or to about 2.0%, from or from about 1.0% to or to about 1.5%, from or from about 1.5% to or to about 3.0%, from or from about 1.5% to or to about 2.5%, from or from about 1.5% to or to about 2.0%, from or from about 2.0% to or to about 3.0%, from or from about 2.0% to or to about 2.5%, or from or from about 2.5% to or to about 3.0% v/v of a 20% albumin solution, e.g., a 20% human albumin solution. In some embodiments, the culture medium comprises and/or is supplemented with from 1.6% to 2.4% v/v of a 20% albumin solution, e.g., a 20% human albumin solution. In some embodiments, the culture medium comprises and/or is supplemented with 2.0% v/v of a 20% albumin solution, e.g., a 20% human albumin solution. In some embodiments, the culture medium comprises about 2.0% v/v of a 20% albumin solution, e.g., a 20% human albumin solution.

In some embodiments, the culture medium comprises and/or is supplemented with from or from about 2 to or to about 6 g/L albumin, e.g., human albumin. In some embodiments, the culture medium comprises and/or is supplemented with from or from about 2 to or to about 5.5, from or from about 2 to or to about 5.0, from or from about 2 to or to about 4.5, from or from about 2 to or to about 4, from or from about 2 to or to about 3.5, from or from about 2 to or to about 3, from or from about 2 to or to about 2.5, from or from about 2.5 to or to about 6, from or from about 2.5 to or to about 5.5, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3 to or to about 6, from or from about 3 to or to about 5.5, from or from about 3 to or to about 5, from or from about 3 to or to about 4.5, from or from about 3 to or to about 4, from or from about 3 to or to about 3.5, from or from about 3.5 to or to about 6, from or from about 3.5 to or to about 5.5, from or from about 3.5 to or to about 5, from or from about 3.5 to or to about 4.5, from or from about 3.5 to or to about 4, from or from about 4 to or to about 6, from or from about 4 to or to about 5.5, from or from about 4 to or to about 5, from or from about 4 to or to about 4.5, from or from about 4.5 to or to about 6, from or from about 4.5 to or to about 5.5, from or from about 4.5 to or to about 5, from or from about 5 to or to about 6, from or from about 5 to or to about 5.5, or from or from about 5.5 to or to about 6 g/L albumin, e.g., human albumin. In some embodiments, the culture medium comprises and/or is supplemented with from 3.2 to 4.8 g/L albumin, e.g., human albumin. In some embodiments, the culture medium comprises 4 g/L albumin, e.g., human albumin. In some embodiments, the culture medium comprises about 4 g/L albumin, e.g., human albumin

In some embodiments, the culture medium is supplemented with Poloxamer 188. In some embodiments, the culture medium comprises and/or is supplemented with from or from about 0.1 to or to about 2.0 g/L Poloxamer 188. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 g/L Poloxamer 188. In some embodiments, the culture medium comprises from 0.8 to 1.2 g/L Poloxamer 188. In some embodiments, the culture medium comprises 1.0 g/L Poloxamer 188. In some embodiments, the culture medium comprises about 1.0 g/L Poloxamer 188.

In some embodiments, the culture medium comprises and/or is supplemented with one or more antibiotics.

A first exemplary culture medium is set forth in Table 1.

TABLE 1
Exemplary Culture Medium #1

| | Exemplary | | Exemplary | |
|---|---|---|---|---|
| Component | Concentration Range | | Concentration | |
| CellgroSCGM liquid medium | undiluted | | undiluted | |
| Human Plasma | 0.8-1.2% | (v/v) | 1.0% | v/v |
| Glutamine | 3.2-4.8 | mM | 4.0 | mM |
| IL-2 | 64-96 | μg/L | 80 | μg/L |

A second exemplary culture medium is set forth in Table 2.

TABLE 2
Exemplary Culture Medium #2

| | Exemplary | | Exemplary | |
|---|---|---|---|---|
| Component | Concentration Range | | Concentration | |
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen Carbonate | 1.6-2.4 | g/L | 2.0 | g/L |
| IL-2 | 64-96 | μg/L | 80 | μg/L |
| Albumin 20% solution | 1.6-2.5% v/v | | 2.0% v/v | |
| | (3.2 to 4.8 g/L) | | (4.0 g/L) | |
| Poloxamer 188 | 0.8-1.2 | g/L | 1.0 | g/L |

### 2. CD3 Binding Antibodies

In some embodiments, the culture medium comprises and/or is supplemented with a CD3 binding antibody or antigen binding fragment thereof. In some embodiments, the CD3 binding antibody or antigen binding fragment thereof is selected from the group consisting of OKT3, UCHT1, and HIT3a, or variants thereof. In some embodiments, the CD3 binding antibody or antigen binding fragment thereof is OKT3 or an antigen binding fragment thereof.

In some embodiments, the CD3 binding antibody or antigen binding fragment thereof and feeder cells are added to the culture vessel before addition of NK cells and/or culture medium.

In some embodiments, the culture medium comprises and/or is supplemented with from or from about 5 ng/ml to or to about 15 ng/mL OKT3. In some embodiments, the culture medium comprises and/or is supplemented with from or from about 5 to or to about 12.5, from or from about 5 to or to about 10, from or from about 5 to or to about 7.5, from or from about 7.5 to or to about 15, from or from about 7.5 to or to about 12.5, from or from about 7.5 to or to about 10, from or from about 10 to or to about 15, from or from about 10 to or to about 12.5, or from or from about 12.5 to or to about 15 ng/mL OKT3. In some embodiments, the culture medium comprises and/or is supplemented with 10 ng/mL OKT3. In some embodiments, the culture medium comprises and/or is supplemented with about 10 ng/ml OKT3.

### 3. Culture Vessels

A number of vessels are consistent with the disclosure herein. In some embodiments, the culture vessel is selected from the group consisting of a flask, a bottle, a dish, a multiwall plate, a roller bottle, a bag, and a bioreactor.

In some embodiments, the culture vessel is treated to render it hydrophilic. In some embodiments, the culture vessel is treated to promote attachment and/or proliferation. In some embodiments, the culture vessel surface is coated with serum, collagen, laminin, gelatin, poy-L-lysine, fibronectin, extracellular matrix proteins, and combinations thereof.

In some embodiments, different types of culture vessels are used for different stages of culturing.

In some embodiments, the culture vessel has a volume of from or from about 100 mL to or to about 1,000 L. In some embodiments, the culture vessel has a volume of or about 125 mL, of or about 250 mL, of or about 500 mL, of or about 1 L, of or about 5 L, of about 10 L, or of or about 20 L.

In some embodiments, the culture vessel is a bioreactor.

In some embodiments, the bioreactor is a rocking bed (wave motion) bioreactor. In some embodiments, the bioreactor is a stirred tank bioreactor. In some embodiments, the bioreactor is a rotating wall vessel. In some embodiments, the bioreactor is a perfusion bioreactor. In some embodiments, the bioreactor is an isolation/expansion automated system. In some embodiments, the bioreactor is an automated or semi-automated bioreactor. In some embodiments, the bioreactor is a disposable bag bioreactor.

In some embodiments, the bioreactor has a volume of from about 100 mL to about 1,000 L. In some embodiments, the bioreactor has a volume of from about 10 L to about 1,000 L. In some embodiments, the bioreactor has a volume of from about 100 L to about 900 L. In some embodiments, the bioreactor has a volume of from about 10 L to about 800 L. In some embodiments, the bioreactor has a volume of from about 10 L to about 700 L, about 10 L to about 600 L, about 10 L to about 500 L, about 10 L to about 400 L, about 10 L to about 300 L, about 10 L to about 200 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 1,000 L, about 20 L to about 900 L, about 20 L to about 800 L, about 20 L to about 700 L, about 20 L to about 600 L, about 20 L to about 500 L, about 20 L to about 400 L, about 20 L to about 300 L, about 20 L to about 200 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 1,000 L, about 30 L to about 900 L, about 30 L to about 800 L, about 30 L to about 700 L, about 30 L to about 600 L, about 30 L to about 500 L, about 30 L to about 400 L, about 30 L to about 300 L, about 30 L to about 200 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 1,000 L, about 40 L to about 900 L, about 40 L to about 800 L, about 40 L to about 700 L, about 40 L to about 600 L, about 40 L to about 500 L, about 40 L to about 400 L, about 40 L to about 300 L, about 40 L to about 200 L, about 40 L to about 100 L, about 40 L to about 90 L, about 40 L to about 80 L, about 40 L to about 70 L, about 40 L to about 60 L, about 40 L to about 50 L, about 50 L to about 1,000 L, about 50 L to about 900 L, about 50 L to about 800 L, about 50 L to about 700 L, about 50 L to about 600 L, about 50 L to about 500 L, about 50 L to about 400 L, about 50 L to about 300 L, about 50 L to about 200 L, about 50 L to about 100 L, about 50 L to about 90 L, about 50 L to about 80 L, about 50 L to about 70 L, about 50 L to about 60 L, about 60 L to about 1,000 L, about 60 L to about 900 L, about 60 L to about 800 L, about 60 L to about 700 L, about 60 L to about 600 L, about 60 L to about 500 L, about 60 L to about 400 L, about 60 L to about 300 L, about 60 L to about 200 L, about 60 L to about 100L, about 60 L to about 90 L, about 60 L to about 80 L, about 60 L to about 70 L, about 70 L to about 1,000 L, about 70 L to about 900 L, about 70 L to about 800 L, about 70 L to about 700 L, about 70 L to about 600 L, about 70 L to about 500 L, about 70 L to about 400 L, about 70 L to about 300 L, about 70 L to about 200 L, about 70 L to about 100 L, about 70 L to about 90 L, about 70 L to about 80 L, about 80 L to about 1,000 L, about 80 L to about 900 L, about 80 L to about 800 L, about 80 L to about 700 L, about 80 L to about 600 L, about 80 L to about 500 L, about 80 L to about 400 L, about 80 L to about 300 L, about 80 L to about 200 L, about 80 L to about 100 L, about 80 L to about 90 L, about 90 L to about 1,000 L, about 90 L to about 900 L, about 90 L to about 800 L, about 90 L to about 700 L, about 90 L to about 600 L, about 90 L to about 500 L, about 90 L to about 400 L, about 90 L to about 300 L, about 90 L to about 200 L, about 90 L to about 100 L, about 100 L to about 1,000 L, about 100 L to about 900 L, about 100 L to about 800 L, about 100 L to about 700 L, about 100 L toa bout 600 L, about 100 L to about 500 L, about 100 L to about 400 L, about 100 L to about 300 L, about 100 L to about 200 L, about 200 L to about 1,000 L, about 200 L to about 900 L, about 200 L to about 800 L, about 200 L to about 700 L, about 200 L to about 600 L, about 200 L to about 500 L, about 200 L to about 400 L, about 200 L to about 300 L, about 300 L to about 1,000 L, about 300 L to about 900 L, about 300 L to about 800 L, about 300 L to about 700 L, about 300 L to about 600 L, about 300 L to about 500 L, about 300 L to about 400 L, about 400 L to about 1,000 L, about 400 L to about 900 L, about 400 L to about 800 L, about 400 L to about 700 L, about 400 L to about 600 L, about 400 L to about 500 L, about 500 L to about 1,000 L, about 500 L to about 900 L, about 500 L to about 800 L, about 500 L to about 700 L, about 500 L to about 600 L, about 600 L to about 1,000 L, about 600 L to about 900 L, about 600 L to about 800 L, about 600 L to about 700 L, about 700 L to about 1,000 L, about 700 L to about 900 L, about 700 L to about 800 L, about 800 L to about 1,000 L, about 800 L to about 900 L, or about 900 L to about 1,000 L. In some embodiments, the bioreactor has a volume of about 50 L.

In some embodiments, the bioreactor has a volume of from 100 mL to 1,000 L. In some embodiments, the bioreactor has a volume of from 10 L to 1,000 L. In some embodiments, the bioreactor has a volume of from 100 L to 900 L. In some embodiments, the bioreactor has a volume of from 10 L to 800 L. In some embodiments, the bioreactor has a volume of from 10 L to 700 L, 10 L to 600 L, 10 L to 500 L, 10 L to 400 L, 10 L to 300 L, 10 L to 200 L, 10 L to 100 L, 10 L to 90 L, 10 L to 80 L, 10 L to 70 L, 10 L to 60 L, 10 L to 50 L, 10 L to 40 L, 10 L to 30 L, 10 L to 20 L, 20 L to 1,000 L, 20 L to 900 L, 20 L to 800 L, 20 L to 700 L, 20 L to 600 L, 20 L to 500 L, 20 L to 400 L, 20 L to 300 L, 20 L to 200 L, 20 L to 100 L, 20 L to 90 L, 20 L to 80 L, 20 L to 70 L, 20 L to 60 L, 20 L to 50 L, 20 L to 40 L, 20 L to 30 L, 30 L to 1,000 L, 30 L to 900 L, 30 L to 800 L, 30 L to 700 L, 30 L to 600 L, 30 L to 500 L, 30 L to 400 L, 30 L to 300 L, 30 L to 200 L, 30 L to 100 L, 30 L to 90 L, 30 L to 80 L, 30 L to 70 L, 30 L to 60 L, 30 L to 50 L, 30 L to 40 L, 40 L to 1,000 L, 40 L to 900 L, 40 L to 800 L, 40 L to 700 L, 40 L to 600 L, 40 L to 500 L, 40 L to 400 L, 40 L to 300 L, 40 L to 200 L, 40 L to 100 L, 40 L to 90 L, 40 L to 80 L, 40 L to 70 L, 40 L to 60 L, 40 L to 50 L, 50 L to 1,000 L, 50 L to 900 L, 50 L to 800 L, 50 L to 700 L, 50 L to 600 L, 50 L to 500 L, 50 L to 400 L, 50 L to 300 L, 50 L to 200 L, 50 L to 100 L, 50 L to 90 L, 50 L to 80 L, 50 L to 70 L, 50 L to 60 L, 60 L to 1,000 L, 60 L to 900 L, 60 L to 800 L, 60 L to 700 L, 60 L to 600 L, 60 L to 500 L, 60 L to 400 L, 60 L to 300 L, 60 L to 200 L, 60 L to 100L, 60 L to 90 L, 60 L to 80 L, 60 L to 70 L, 70 L to 1,000 L, 70 L to 900 L, 70 L to 800 L, 70 L to 700 L, 70 L to 600 L, 70 L to 500 L, 70 L to 400 L, 70 L to 300 L, 70 L to 200 L, 70 L to 100 L, 70 L to 90 L, 70 L to 80 L, 80 L to 1,000 L, 80 L to 900 L, 80 L to 800 L, 80 L to 700 L, 80 L to 600 L, 80 L to 500 L, 80 L to 400 L, 80 L to 300 L, 80 L to 200 L, 80 L to 100 L, 80 L to 90 L, 90 L to 1,000 L, 90 L to 900 L, 90 L to 800 L, 90 L to 700 L, 90 L to 600 L, 90 L to 500 L, 90 L to 400 L, 90 L to 300 L, 90 L to 200 L, 90 L to 100 L, 100 L to 1,000 L, 100 L to 900 L, 100 L to 800 L, 100 L to 700 L, 100 L to 600 L, 100 L to 500 L, 100 L to 400 L, 100 L to 300 L, 100 L to 200 L, 200 L to 1,000 L, 200 L to 900 L, 200 L to 800 L, 200 L to 700 L, 200 L to 600 L, 200 L to 500 L, 200 L to 400 L, 200 L to 300 L, 300 L to 1,000 L, 300 L to 900 L, 300 L to 800 L, 300 L to 700 L, 300 L to 600 L, 300 L to 500 L, 300 L to 400 L, 400 L to 1,000 L, 400 L to 900 L, 400 L to 800 L, 400 L to 700 L, 400 L to 600 L, 400 L to 500 L, 500 L to 1,000 L, 500 L to 900 L, 500 L to 800 L, 500 L to 700 L, 500 L to 600 L, 600 L to 1,000 L, 600 L to 900 L, 600 L to 800 L, 600 L to 700 L, 700 L to 1,000 L, 700 L to 900 L, 700 L to 800 L, 800 L to 1,000 L, 800 L to 900 L, or 900 L to 1,000 L. In some embodiments, the bioreactor has a volume of 50 L.

### 4. Cell Expansion and Stimulation

In some embodiments, the natural killer cell source, e.g., single unit of cord blood, is co-cultured with feeder cells to produce expanded and stimulated NK cells.

In some embodiments, the co-culture is carried out in a culture medium described herein, e.g., exemplary culture medium #1 (Table 1) or exemplary culture medium #2 (Table 2).

In some embodiments, the natural killer cell source, e.g., single unit of cord blood, comprises from or from about 1×10⁷ to or to about 1×10⁹ total nucleated cells prior to expansion. In some embodiments, the natural killer cell source, e.g., single unit of cord blood, comprises from or from about 1×10⁸ to or to about 1.5×10⁸ total nucleated cells prior to expansion. In some embodiments, the natural killer cell source, e.g., single unit of cord blood, comprises 1×10⁸ total nucleated cells prior to expansion. In some embodiments, the natural killer cell source, e.g., single unit of cord blood, comprises about 1×10⁸ total nucleated cells prior to expansion. In some embodiments, the natural killer cell source, e.g., single unit of cord blood, comprises 1×10⁹ total nucleated cells prior to expansion. In some embodiments, the natural killer cell source, e.g., single unit of cord blood, comprises about 1×10⁹ total nucleated cells prior to expansion.

In some embodiments, cells from the co-culture of the natural killer cell source, e.g., single unit of cord blood and feeder cells are harvested and frozen, e.g., in a cryopreservation composition described herein. In some embodiments, the frozen cells from the co-culture are an infusion-ready drug product. In some embodiments, the frozen cells from the co-culture are used as a master cell bank (MCB) from which to produce an infusion-ready drug product, e.g., through one or more additional co-culturing steps, as described herein. Thus, for example, a natural killer cell source can be expanded and stimulated as described herein to produce expanded and stimulated NK cells suitable for use in an infusion-ready drug product without generating any intermediate products. A natural killer cell source can also be expanded and stimulated as described herein to produce an intermediate product, e.g., a first master cell bank (MCB). The first MCB can be used to produce expanded and stimulated NK cells suitable for use in an infusion-ready drug product, or, alternatively, be used to produce another intermediate product, e.g., a second MCB. The second MCB can be used to produce expanded and stimulated NK cells suitable for an infusion-ready drug product, or alternatively, be used to produce another intermediate product, e.g., a third MCB, and so on.

In some embodiments, the ratio of feeder cells to cells of the natural killer cell source or MCB cells inoculated into the co-culture is from or from about 1:1 to or to about 4:1. In some embodiments, the ratio of feeder cells to cells of the natural killer cell source or MCB cells is from or from about 1:1 to or to about 3.5:1, from or from about 1:1 to or to about 3:1, from or from about 1:1 to or to about 2.5:1, from or from about 1.1 to or to about 2:1, from or from about 1:1 to or to about 1.5:1, from or from about 1.5:1 to or to about 4:1, from or from about 1.5:1 to or to about 3.5:1, from or from about 1.5:1 to or to about 3:1, from or from about 1.5:1 to or to about 2.5:1, from or from about 1.5:1 to or to about 2:1, from or from about 2:1 to or to about 4:1, from or from about 2:1 to or to about 3.5:1, from or from about 2:1 to or to about 3:1, from or from about 2:1 to or to about 2.5:1, from or from about 2.5:1 to or to about 4:1, from or from about 2.5:1 to or to about 3.5:1, from or from about 2.5:1 to or to about 3:1, from or from about 3:1 to or to about 4:1, from or from about 3:1 to or to about 3.5:1, or from or from about 3.5:1 to or to about 4:1. In some embodiments, the ratio of feeder cells to cells of the natural killer cell source or MCB inoculated into the co-culture is 2.5:1. In some embodiments, the ratio of feeder cells to cells of the natural killer cell source or MCB inoculated into the co-culture is about 2.5:1.

In some embodiments, the co-culture is carried out in a disposable culture bag, e.g., a 1L disposable culture bag. In some embodiments, the co-culture is carried out in a bioreactor, e.g., a 50L bioreactor. In some embodiments, culture medium is added to the co-culture after the initial inoculation.

In some embodiments, the co-culture is carried out for 1, 2, 3, 4, 5, 6, 7, 8, 9, 10, 11, 12, 13, 14, 15, 16, 17, 18, 19, 20, 21, 22, 23, 24 or more days. In some embodiments, the co-culture is carried out for a maximum of 16 days.

In some embodiments, the co-culture is carried out at 37° C. or about 37° C.

In some embodiments, the co-culture is carried out at pH 7.9 or about pH 7.9.

In some embodiments, the co-culture is carried out at a dissolved oxygen (DO) level of 50% or more.

In some embodiments, exemplary culture medium #1 (Table 1) is used to produce a MCB and exemplary culture medium #2 (Table 2) is used to produce cells suitable for an infusion-ready drug product.

In some embodiments, the co-culture of the natural killer cell source, e.g., single unit of cord blood, with feeder cells yields from or from about 50×10⁸ to or to about 50×10¹² cells, e.g., MCB cells or infusion-ready drug product cells. In some embodiments, the expansion yields from or from about 50×10⁸ to or to about 25×10¹⁰, from or from about 10×10⁸ to or to about 1×10¹⁰, from or from about 50×10⁸ to or to about 75×10⁹, from or from about 50×10⁸ to or to about 50×10⁹, from or from about 50×10⁸ to or to about 25×10⁹, from or from about 50×10⁸ to or to about 1×10⁹, from or from about 50×10⁸ to or to about 75×10⁸, from or from about 75×10⁸ to or to about 50×10¹⁰, from or from about 75×10⁸ to or to about 25×10¹⁰, from or from about 75×10⁸ to or to about 1×10¹⁰, from or from about 75×10⁸ to or to about 75×10⁹, from or from about 75×10⁸ to or to about 50×10⁹, from or from about 75×10⁸ to or to about 25×10⁹, from or from about 75×10⁸ to or to about 1×10⁹, from or from about 1×10⁹ to or to about 50×10¹⁰, from or from about 1×10⁹ to or to about 25×10¹⁰, from or from about 1×10⁹ to or to about 1×10¹⁰, from or from about 1×10⁹ to or to about 75×10⁹, from or from about 1×10⁹ to or to about 50×10⁹, from or from about 1×10⁹ to or to about 25×10⁹, from or from about 25×10⁹ to or to about 50×10¹⁰, from or from about 25×10⁹ to or to about 25×10¹⁰, from or from about 25×10⁹ to or to about 1×10¹⁰, from or from about 25×10⁹ to or to about 75×10⁹, from or from about 25×10⁹ to or to about 50×10⁹, from or from about 50×10⁹ to or to about 50×10¹⁰, from or from about 50×10⁹ to or to about 25×10¹⁰, from or from about 50×10⁹ to or to about 1×10¹⁰, from or from about 50×10⁹ to or to about 75×10⁹, from or from about 75×10⁹ to or to about 50×10¹⁰, from or from about 75×10⁹ to or to about 25×10¹⁰, from or from about 75×10⁹ to or to about 1×10¹⁰, from or from about 1×10¹⁰ to or to about 50×10¹⁰, from or from about 1×10¹⁰ to or to about 25×10¹⁰, or from or from about 25×10¹⁰ to or to about 50×10¹⁰ cells, e.g., MCB cells or infusion-ready drug product cells.

In some embodiments, the expansion yields from or from about 60 to or to about 100 vials, each comprising from or from about 600 million to or to about 1 billion cells, e.g., MCB cells or infusion-ready drug product cells. In some embodiments, the expansion yields 80 or about 80 vials, each comprising or consisting of 800 million or about 800 million cells, e.g., MCB cells or infusion-ready drug product cells.

In some embodiments, the expansion yields from or from about a 100 to or to about a 500 fold increase in the number of cells, e.g., the number of MCB cells relative to the number of cells, e.g., NK cells, in the natural killer cell source. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of MCB cells relative to the number of cells, e.g., NK cells, in the natural killer cell source.

In some embodiments, the expansion yields from or from about a 100 to or to about a 70,000 fold increase in the number of cells, e.g., the number of MCB cells relative to the number of cells, e.g., NK cells, in the natural killer cell source. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of MCB cells relative to the number of cells, e.g., NK cells, in the natural killer cell source.

In some embodiments, the co-culture of the MCB cells and feeder cells yields from or from about 500 million to or to about 1.5 billion cells, e.g., NK cells suitable for use in an MCB and/or in an infusion-ready drug product. In some embodiments, the co-culture of the MCB cells and feeder cells yields from or from about 500 million to or to about 1.5 billion, from or from about 500 million to or to about 1.25 billion, from or from about 500 million to or to about 1 billion, from or from about 500 million to or to about 750 million, from or from about 750 million to or to about 1.5 billion, from or from about 500 million to or to about 1.25 billion, from or from about 750 million to or to about 1 billion, from or from about 1 billion to or to about 1.5 billion, from or from about 1 billion to or to about 1.25 billion, or from or from about 1.25 billion to or to about 1.5 billion cells, e.g., NK cells suitable for use in an MCB and/or an infusion-ready drug product.

In some embodiments, the co-culture of the MCB cells and feeder cells yields from or from about 50 to or to about 150 vials of cells, e.g., infusion-ready drug product cells, each comprising from or from about 750 million to or to about 1.25 billion cells, e.g., NK cells suitable for use in an MCB and/or an infusion-ready drug product. In some embodiments, the co-culture of the MCB cells and feeder cells yields 100 or about 100 vials, each comprising or consisting of 1 billion or about 1 billion cells, e.g., NK cells suitable for use in an MCB and/or an infusion-ready drug product.

In some embodiments, the expansion yields from or from about a 100 to or to about a 500 fold increase in the number of cells, e.g., the number of NK cells suitable for use in an MCB and/or an infusion-ready drug product relative to the number of starting MCB cells. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of NK cells suitable for use in an MCB and/or an infusion-ready drug product relative to the number of starting MCB cells.

In some embodiments, the expansion yields from or from about a 100 to or to about a 70,000 fold increase in the number of cells, e.g., the number of NK cells suitable for use in an MCB and/or an infusion-ready drug product relative to the number of starting MCB cells. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of NK cells suitable for use in an MCB and/or an infusion-ready drug product relative to the number of starting MCB cells.

In embodiments where the cells are engineered during expansion and stimulation, as described herein, not all of the expanded and stimulated cells will necessarily be engineered successfully, e.g., transduced successfully, e.g., transduced successfully with a vector comprising a heterologous protein, e.g., a heterologous protein comprising a CAR and/or IL-15 as described herein. Thus, the methods described herein can further comprise sorting engineered cells, e.g., engineered cells described herein, away from non-engineered cells.

In some embodiments, the engineered cells, e.g., transduced cells, are sorted from the non-engineered cells, e.g., the non-transduced cells using a reagent specific to an antigen of the engineered cells, e.g., an antibody that targets an antigen of the engineered cells but not the non-engineered cells. In some embodiments, the antigen of the engineered cells is a component of a CAR, e.g., a CAR described herein.

Systems for antigen-based cell separation of cells are available commercially, e.g., the CliniMACS® sorting system (Miltenyi Biotec).

In some embodiments, the engineered cells, e.g., transduced cells, are sorted from the non-engineered cells, e.g., the non-transduced cells using flow cytometry.

In some embodiments, the sorted engineered cells are used as an MCB. In some embodiments, the sorted engineered cells are used as a component in an infusion-ready drug product.

In some embodiments, the engineered cells, e.g., transduced cells, are sorted from the non-engineered cells, e.g., the non-transduced cells using a microfluidic cell sorting method. Microfluidic cell sorting methods are described, for example, in Dalili et al., “A Review of Sorting, Separation and Isolation of Cells and Microbeads for Biomedical Applications: Microfluidic Approaches,” Analyst 144:87 (2019).

In some embodiments, from or from about 1% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein. In some embodiments, from or from about 1% to or to about 90%, from or from about 1% to or to about 80%, from or from about 1% to or to about 70%, from or from about 1% to or to about 60%, from or from about 1% to or to about 50%, from or from about 1% to or to about 40%, from or from about 1% to or to about 30%, from or from about 1% to or to about 20%, from or from about 1% to or to about 10%, from or from about 1% to or to about 5%, from or from about 5% to or to about 99%, from or from about 5% to or to about 90%, from or from about 5% to or to about 80%, from or from about 5% to or to about 70%, from or from about 5% to or to about 60%, from or from about 5% to or to about 50%, from or from about 5% to or to about 40%, from or from about 5% to or to about 30%, from or from about 5% to or to about 20%, from or from about 5% to or to about 10%, from or from about 10% to or to about 99%, from or from about 10% to or to about 90%, from or from about 10% to or to about 80%, from or from about 10% to or to about 70%, from or from about 10% to or to about 60%, from or from about 10% to or to about 50%, from or from about 10% to or to about 40%, from or from about 10% to or to about 30%, from or from about 10% to or to about 20%, from or from about 20% to or to about 99%, from or from about 20% to or to about 90%, from or from about 20% to or to about 80%, from or from about 20% to or to about 70%, from or from about 20% to or to about 60%, from or from about 20% to or to about 50%, from or from about 20% to or to about 40%, from or from about 20% to or to about 30%, from or from about 30% to or to about 99%, from or from about 30% to or to about 90%, from or from about 30% to or to about 80%, from or from about 30% to or to about 70%, from or from about 30% to or to about 60%, from or from about 30% to or to about 50%, from or from about 30% to or to about 40%, from or from about 40% to or to about 99%, from or from about 40% to or to about 90%, from or from about 40% to or to about 80%, from or from about 40% to or to about 70%, from or from about 40% to or to about 70%, from or from about 40% to or to about 60%, from or from about 40% to or to about 50%, from or from about 50% to or to about 99%, from or from about 50% to or to about 90%, from or from about 50% to or to about 80%, from or from about 50% to or to about 70%, from or from about 50% to or to about 60%, from or from about 60% to or to about 99%, from or from about 60% to or to about 90%, from or from about 60% to or to about 80%, from or from about 60% to or to about 70%, from or from about 70% to or to about 99%, from or from about 70% to or to about 90%, from or from about 70% to or to about 80%, from or from about 80% to or to about 99%, from or from about 80% to or to about 90%, or from or from about 90% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein.

In some embodiments, frozen cells of a first or second MCB are thawed and cultured. In some embodiments, a single vial of frozen cells of the first or second MCB e.g., a single vial comprising 800 or about 800 million cells, e.g., first or second MCB cells, are thawed and cultured. In some embodiments, the frozen first or second MCB cells are cultured with additional feeder cells to produce cells suitable for use either as a second or third MCB or in an infusion-ready drug product. In some embodiments, the cells from the co-culture of the first or second MCB are harvested and frozen.

In some embodiments, the cells from the co-culture of the natural killer cell source, a first MCB, or a second MCB are harvested, and frozen in a cryopreservation composition, e.g., a cryopreservation composition described herein. In some embodiments, the cells are washed after harvesting. Thus, provided herein is a pharmaceutical composition comprising activated and stimulated NK cells, e.g., activated and stimulated NK cells produced by the methods described herein, e.g., harvested and washed activated and stimulated NK cells produced by the methods described herein and a cryopreservation composition, e.g., a cryopreservation composition described herein.

In some embodiments, the cells are mixed with a cryopreservation composition, e.g., as described herein, before freezing. In some embodiments, the cells are frozen in cryobags. In some embodiments, the cells are frozen in cryovials.

In some embodiments, the method further comprises isolating NK cells from the population of expanded and stimulated NK cells.

### 5. Engineering

In some embodiments, the method further comprises engineering NK cell(s), e.g., to express a heterologous protein, e.g., a heterologous protein described herein, e.g., a heterologous protein comprising a CAR and/or IL-15.

In some embodiments, engineering the NK cell(s) to express a heterologous protein described herein comprises transforming or transfecting, e.g., stably transforming or transfecting the NK cells with a vector comprising a polynucleic acid encoding a heterologous protein described herein. Suitable vectors are described herein.

In some embodiments, engineering the NK cell(s) to express a heterologous protein described herein comprises introducing the heterologous protein via gene editing (e.g., zinc finger nuclease (ZFN) gene editing, ARCUS gene editing, CRISPR-Cas9 gene editing, or megaTAL gene editing) combined with adeno-associated virus (AAV) technology.

In some embodiments, the NK cell(s) are engineered to express a heterologous protein described herein, e.g., during or after culturing the composition in a medium comprising feeder cells. For example, in some cases, engineering (e.g., transduction) occurs during the expansion and stimulation process described herein, e.g., during co-culturing NK cell source(s) and feeder cell(s) as described herein, e.g., at day 1, 2, 3, 4, 5, 6, 7, 8, 9, 10, 11, 12, 13, 14, 15, 16, 17, or 18 of co-culturing.

In some embodiments, the method further comprises engineering NK cell(s), e.g., to express, over-express, knock-out, or knock-down gene(s) or gene product(s).

In some embodiments, the natural killer cells are not genetically engineered.

In some embodiments, the NK cell(s) are engineered (e.g., transduced) in a culture medium supplemented with a stimulating factor (e.g., as described herein). In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-21. In some embodiments, the cytokine is IL-2. In some embodiments, the cytokines are a combination of IL-2 and IL-21. In some embodiments, the cytokines are a combination of IL-2, IL-18, and IL-21.

In some embodiments, the stimulating factor is added to the culture medium at the time of engineering (e.g., transduction). In some embodiments, the stimulating factor is added to the culture medium after the time of engineered (e.g., transducing), e.g., from 1 to 48 hours after engineering, e.g., from 1 to 36, 1 to 24, 1 to 12, 12 to 28, 12 to 36, 12 to 24, 24 to 48, 24 to 36, or 36 to 48 hours after engineering. In some embodiments, the stimulating factor is added to the culture medium both at the time of transduction and after the time of engineering (e.g., from 1 to 48 hours after transduction).

In some embodiments, the culture is supplemented with the stimulating factor after culturing in a medium comprising feeder cells. Thus, in some cases, the culture medium will contain feeder cells at the time of engineering (e.g., transduction). In some cases, the feeder cells are removed from the culture prior to supplementation with the stimulating factor. In some cases, the feeder cells are not removed from the culture prior to supplementation with the stimulating factor. In some cases, no additional feeder cells are added to the culture during engineering, whether or not any residual feeder cells are removed. In some cases, both additional feeder cells and a stimulating factor are added to the culture during engineering. In some cases, additional feeder cells are not added to the culture during engineering but stimulating factors are added to the culture during engineering.

### E. Properties of Expanded and Stimulated NK Cells

After having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3-cells.

In some embodiments, the expanded and stimulated NK cells are not genetically engineered.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3(+) depleted, e.g., by gating on CD56+CD3− expression, but no other surface protein expression selection is carried out during expansion and stimulation.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% CD94+ (KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+ (IL2RB) cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+ NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+ NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., Jiao et al., “CD38: Targeted Therapy in Multiple Myeloma and Therapeutic Potential for Solid Cancers,” Expert Opinion on Investigational Drugs 29(11):1295-1308 (2020). Yet, when an anti-CD38 antibody is administered with NK cells, because NK cells naturally express CD38, they are at risk for increased fratricide. The NK cells expanded and stimulated by the methods described herein, however, express low levels of CD38 and, therefore, overcome the anticipated fratricide. While other groups have resorted to engineering methods such as genome editing to reduce CD38 expression (see, e.g., Gurney et al., “CD38 Knockout Natural Killer Cells Expressing an Affinity Optimized CD38 Chimeric Antigen Receptor Successfully Target Acute Myeloid Leukemia with Reduced Effector Cell Fratricide,” Haematologica doi: 10.3324/haematol.2020.271908 (2020), the NK cells expanded and stimulated by the methods described herein express low levels of CD38 without the need for genetic engineering, which provides a surprising and unexpected benefits, e.g., for treating CD38+ cancers with the NK cells expanded and stimulated as described herein, e.g., in combination with a CD38 antibody.

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+ NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+ NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+ (KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+ (IL2RB) cells.

In some embodiments, feeder cells do not persist in the expanded and stimulated NK cells, though, residual signature of the feeder cells may be detected, for example, by the presence of residual cells (e.g., by detecting cells with a particular surface protein expression) or residual nucleic acid and/or proteins that are expressed by the feeder cells.

For example, in some cases, the methods described herein include expanding and stimulating natural killer cells using engineered feeder cells, e.g., eHuT-78 feeder cells described above, which are engineered to express sequences that are not expressed by cells in the natural killer cell source, including the natural killer cells. For example, the engineered feeder cells can be engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

While these feeder cells may not persist in the expanded and stimulated NK cells, the expanded and stimulated NK cells may retain detectable residual amounts of cells, proteins, and/or nucleic acids from the feeder cells. Thus, their residual presence in the expanded and stimulated NK cells may be detected, for example, by detecting the cells themselves (e.g., by flow cytometry), proteins that they express, and/or nucleic acids that they express.

Thus, also described herein is a population of expanded and stimulated NK cells comprising residual feeder cells (live cells or dead cells) or residual feeder cell cellular impurities (e.g., residual feeder cell proteins or portions thereof, and/or genetic material such as a nucleic acid or portion thereof). In some cases, the expanded and stimulated NK cells comprise more than 0% and, but 0.3% or less residual feeder cells, e.g., eHuT-78 feeder cells.

In some cases, the expanded and stimulated NK cells comprise residual feeder cell nucleic acids, e.g., encoding residual 4-1BBL (UniProtKB P41273, SEQ ID NO: 1), membrane bound IL-21 (SEQ ID NO: 2), and/or mutant TNFalpha (SEQ ID NO: 3) or portion(s) thereof. In some cases, the membrane bound IL-21 comprises a CD8 transmembrane domain

In some cases, the expanded and stimulated NK cells comprise a % residual feeder cells of more than 0% and less than or equal to 0.2%, as measured, e.g., by the relative proportion of a feeder cell specific protein or nucleic acid sequence (that is, a protein or nucleic acid sequence not expressed by the natural killer cells) in the sample. For example, by qPCR, e.g., as described herein.

In some embodiments, the residual feeder cells are CD4(+) T cells. In some embodiments, the residual feeder cells are engineered CD4(+) T cells. In some embodiments, the residual feeder cell cells are engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof. Thus, in some cases, the feeder cell specific protein is 4-1BBL (UniProtKB P41273, SEQ ID NO: 1), membrane bound IL-21 (SEQ ID NO: 2), and/or mutant TNFalpha (SEQ ID NO: 3). And, therefore, the feeder cell specific nucleic acid is a nucleic acid encoding 4-1BBL (UniProtKB P41273, SEQ ID NO: 1), membrane bound IL-21 (SEQ ID NO: 2), and/or mutant TNFalpha (SEQ ID NO: 3), or portion thereof. In some cases, the membrane bound IL-21 comprises a CD8 transmembrane domain.

A wide variety of different methods can be used to analyze and detect the presence of nucleic acids or protein gene products in a biological sample. As used herein, “detecting” can refer to a method used to discover, determine, or confirm the existence or presence of a compound and/or substance (e.g., a cell, a protein and/or a nucleic acid). In some embodiments, a detecting method can be used to detect a protein. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein (Jungblut et al., J Biotechnol. 31; 41(2-3):111-20 (1995); Franco et al., Eur J Morphol. 39(1):3-25 (2001)). In some embodiments, a detecting method can be used to detect a nucleic acid (e.g., DNA and/or RNA). In some embodiments, detecting can include Northern blot analysis, nuclease protection assays (NPA), in situ hybridization, or reverse transcription-polymerase chain reaction (RT-PCR) (Raj et al., Nat. Methods 5, 877-879 (2008); Jin et al., J Clin Lab Anal. 11(1):2-9 (1997); Ahmed, J Environ Sci Health C Environ Carcinog Ecotoxicol Rev. 20(2):77-116 (2002)).

Thus, also described herein, are methods for detecting a population of expanded and stimulated NK cells, e.g., expanded and stimulated using the methods described herein, that have been co-cultured with engineered feeder cells, e.g., eHuT-78 feeder cells described herein.

### II. NATURAL KILLER CELL ENGINEERING

In some embodiments, the natural killer cells are engineered, e.g., to produce CAR-NK(s) and/or IL-15 expressing NK(s).

In some embodiments, the natural killer cells are engineered, e.g., transduced, during expansion and stimulation, e.g., expansion and stimulation described herein. In some embodiments, the natural killer cells are engineered during expansion and stimulation, e.g., during production of a MCB, as described herein. In some embodiments, the natural killer cells are engineered during expansion and stimulation, e.g., during production of NK cells suitable for use in an injection-ready drug product and/or during production of a MCB, as described above. Thus, in some embodiments, the NK cell(s) are host cells and provided herein are NK host cell(s) expressing a heterogeneous protein, e.g., as described herein.

In some embodiments, the natural killer cells are engineered prior to expansion and stimulation. In some embodiments, the natural killer cells are engineered after expansion and stimulation.

In some embodiments, the NK cells are engineered by transducing with a vector. Suitable vectors are described herein, e.g., lentiviral vectors, e.g., a lentiviral vectors comprising a heterologous protein, e.g., as described herein. In some embodiments, the NK cells are transduced during production of a first MCB, as described herein.

In some embodiments, the NK cell(s) are transduced at a multiplicity of infection of from or from about 1 to or to about 40 viral particles per cell. In some embodiments, the NK cell(s) are transduced at a multiplicity of infection of or of about 1, of or of about 5, of or of about 10, of or of about 15, of or of about 20, of or of about 25, of or of about 30, of or of about 35, or of or of about 40 viral particles per cell.

### A. Chimeric Antigen Receptors

In some embodiments, the heterologous protein is a fusion protein, e.g., a fusion protein comprising a chimeric antigen receptor (CAR) is introduced into the NK cell, e.g., during the expansion and stimulation process.

In some embodiments, the CAR comprises one or more of: a signal sequence, an extracellular domain, a hinge, a transmembrane domain, and one or more intracellular signaling domain sequences. In some embodiments, the CAR further comprises a spacer sequence.

In some embodiments, the CAR comprises (from N- to C-terminal): a signal sequence, an extracellular domain, a hinge, a spacer, a transmembrane domain, a first signaling domain sequence, a second signaling domain sequence, and a third signaling domain sequence.

In some embodiments, the CAR comprises (from N- to C-terminal): a signal sequence, an extracellular domain, a hinge, a transmembrane domain, a first signaling domain sequence, a second signaling domain sequence, and a third signaling domain sequence.

In some embodiments the extracellular domain comprises an antibody or antigen-binding portion thereof.

In some embodiments, one or more of the intracellular signaling domain sequence(s) is a CD28 intracellular signaling sequence. In some embodiments, the CD28 intracellular signaling sequence comprises or consists of SEQ ID NO: 5.

In some embodiments, one or more of the intracellular signaling domain sequence(s) is an OX40L signaling sequence. See, e.g., Matsumura et al., “Intracellular Signaling of gp34, the OX40 Ligand: Induction of c-jun and c-fos mRNA Expression Through gp34 upon Binding of Its Receptor, OX40,” J. Immunol 163:3007-11 (1999), which is hereby incorporated by reference in its entirety. In some embodiments, the OX40L signaling sequence comprises or consists of SEQ ID NO: 8, SEQ ID NO: 9, or SEQ ID NO: 10.

In some embodiments, one or more of the intracellular signaling sequence(s) is a CD3ζ intracellular signaling domain sequence. In some embodiments, the CD3ζ intracellular signaling sequence comprises of consists of SEQ ID NO: 13.

In some embodiments, the CAR comprises a CD28 intracellular signaling sequence (SEQ ID NO: 5), an OX40L intracellular signaling sequence (SEQ ID NO: 8), and a CD3ζ intracellular signaling sequence (SEQ ID NO: 13).

In some embodiments, the CAR comprises an intracellular signaling domain comprising or consisting of SEQ ID NO: 25.

In some embodiments, the CAR does not comprise an OX40L intracellular signaling domain sequence.

In some embodiments, the CAR comprises a CD28 intracellular signaling sequence (SEQ ID NO: 5), and a CD3ζ intracellular signaling sequence (SEQ ID NO: 13), but not an OX40L intracellular signaling domain sequence.

### B. IL-15

In some embodiments, the NK cell is engineered to express IL-15, e.g., human IL-15 (UniProtKB #P40933; NCBI Gene ID #3600), e.g., soluble human IL-15 or an ortholog thereof, or a variant of any of the foregoing. In some embodiments, the IL-15 is expressed as part of a fusion protein further comprising a cleavage site. In some embodiments, the IL-15 is expressed as part of a polyprotein comprising a self-cleaving peptide such as T2A ribosomal skip sequence site See, e.g., Radcliffe & Mitrophanous, “Multiple Gene Products from a Single Vector: ‘Self-Cleaving’ 2A Peptides,” Gene Therapy 11:1673-4 (2004); see also Liu et al., “Systematic Comparison of 2A Peptides for Cloning Multi-Genes in a Polycistronic Vector,” Scientific Reports 7(1):2193 (2017).

In some embodiments, the IL-15 comprises or consists of SEQ ID NO: 22.

In some embodiments, the self-cleaving peptide is a 2A self-cleaving peptide. In some embodiments, the self-cleaving peptide is a T2A, P2A, E2A, or F2A self-cleaving peptide. In some embodiments, the self-cleaving peptide comprises SEQ ID NO: 16. In some embodiments, the self-cleaving peptide comprises or consists of SEQ ID NO: 16, SEQ ID NO: 17, SEQ ID NO: 19, SEQ ID NO: 20, or SEQ ID NO: 21.

In some embodiments, the T2A cleavage site comprises or consists of SEQ ID NO: 17.

In some embodiments, the IL-15 is expressed as part of a fusion protein comprising a CAR, e.g., a CAR described herein.

In some embodiments, the fusion protein comprises (oriented from N-terminally to C-terminally): a CAR comprising, a cleavage site, and IL-15.

In some embodiments, the fusion protein comprises SEQ ID NO: 26.

### C. Inhibitory Receptors

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRB1, PD-1, IRp60, Siglec-7, LAIR-1, and combinations thereof.

### D. Polynucleic Acids, Vectors, and Host Cells

Also provided herein are polynucleic acids encoding the fusion protein(s) or portions thereof, e.g., the polynucleotide sequences encoding the polypeptides described herein, as shown in the Table of sequences provided herein

Also provided herein are vector(s) comprising the polynucleic acids, and cells, e.g., NK cells, comprising the vector(s).

In some embodiments, the vector is a lentivirus vector. See, e.g., Milone et al., “Clinical Use of Lentiviral Vectors,” Leukemia 32:1529-41 (2018). In some embodiments, the vector is a retrovirus vector. In some embodiments, the vector is a gamma retroviral vector. In some embodiments, the vector is a non-viral vector, e.g., a piggyback non-viral vector (PB transposon, see, e.g., Wu et al., “piggyback is a Flexible and Highly Active Transposon as Compared to Sleeping Beauty, Tol2, and Mos1 in Mammalian Cells,” PNAS 103(41):15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52(4):355-380 (2017)), or an mRNA vector.

### III. PHARMACEUTICAL COMPOSITIONS

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein, e.g., the CAR-NKs described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### IV. METHODS OF TREATMENT

The NK cells described herein, e.g., the CAR-NK cells described herein, find use for treating cancer or other proliferative disorders.

Thus, also provided herein are methods of treating a patient suffering from a disorder, e.g., a disorder associated with a cancer, comprising administering the NK cells, e.g., the NK cells described herein, e.g., the CAR-NK cells described herein.

Also provided herein are methods of preventing, reducing and/or inhibiting the recurrence, growth, proliferation, migration and/or metastasis of a cancer cell or population of cancer cells in a subject in need thereof, comprising administering the NK cells, e.g., the NK cells described herein, e.g., the CAR-NK cells described herein.

Also provided herein are methods of enhancing, improving, and/or increasing the response to an anticancer therapy in a subject in need thereof, comprising administering the NK cells, e.g., the NK cells described herein, e.g., the CAR-NK cells described herein.

Also provided herein are methods for inducing the immune system in a subject in need thereof comprising administering the NK cells, e.g., the NK cells described herein, e.g., the CAR-NK cells described herein.

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. Generally, the methods include administering a therapeutically effective amount of a treatment as described herein, to a subject who is in need of, or who has been determined to be in need of, such treatment. In some embodiments, the methods include administering a therapeutically effective amount of a treatment comprising NK cells, e.g., CAR-NK cells described herein.

As used herein, the terms “treatment,” “treat,” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition”, as it relates to cancer and/or cancer cell proliferation, refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

### A. Disorders

Methods and manufactured compositions disclosed herein find use in targeting a number of disorders, such as cellular proliferative disorders. A benefit of the approaches herein is that allogenic cells are used to target specific cells. Unlike previous therapies, such as chemo or radiotherapy, using the approaches and pharmaceutical compositions herein, one is able to specifically target cells exhibiting detrimental proliferative activity, potentially without administering a systemic drug or toxin that impacts proliferating cells indiscriminately.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the cancer is selected from the group consisting of: acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, and Wilms tumor.

In some embodiments, the cancer is a solid tumor.

In some embodiments, the cancer is metastatic.

### B. Patients

Suitable patients for the compositions and methods herein include those who are suffering from, who have been diagnosed with, or who are suspected of having a cellular proliferative and/or differentiative disorder, e.g., a cancer. Patients subjected to technology of the disclosure herein generally respond better to the methods and compositions herein, in part because the pharmaceutical compositions are allogeneic and target cells identified by the antigen binding domain, rather than targeting proliferating cells generally. As a result, there is less off-target impact and the patients are more likely to complete treatment regimens without substantial detrimental off-target effects.

In some embodiments, the methods of treatment provided herein may be used to treat a subject (e.g., human, monkey, dog, cat, mouse) who has been diagnosed with or is suspected of having a cellular proliferative and/or differentiative disorder, e.g., a cancer. In some embodiments, the subject is a mammal. In some embodiments, the subject is a human.

In some embodiments, the mammal is selected from the group consisting of an armadillo, an ass, a bat, a bear, a beaver, a cat, a chimpanzee, a cow, a coyote, a deer, a dog, a dolphin, an elephant, a fox, a panda, a gibbon, a giraffe, a goat, a gopher, a hedgehog, a hippopotamus, a horse, a humpback whale, a jaguar, a kangaroo, a koala, a leopard, a lion, a llama, a lynx, a mole, a monkey, a mouse, a narwhal, an orangutan, an orca, an otter, an ox, a pig, a polar bear, a porcupine, a puma, a rabbit, a raccoon, a rat, a rhinoceros, a sheep, a squirrel, a tiger, a walrus, a weasel, a wolf, a zebra, a goat, a horse, and combinations thereof.

In some embodiments, the mammal is a human.

As used herein, a subject refers to a mammal, including, for example, a human.

The subject, e.g., the human subject, can be a child, e.g., from or from about 0 to or to about 14 years in age. The subject can be a youth, e.g., from or from about 15 to or to about 24 years in age. The subject can be an adult, e.g., from or from about 25 to or to about 64 years in age. The subject can be a senior, e.g, 65+ years in age.

In some embodiments, the subject may be a human who exhibits one or more symptoms associated with a cellular proliferative and/or differentiative disorder, e.g., a cancer, e.g., a tumor. Any of the methods of treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. The methods of treatment described herein can promote reduction or retraction of a tumor, decrease or inhibit tumor growth or cancer cell proliferation, and/or induce, increase or promote tumor cell killing. In some embodiments, the subject is in cancer remission. The methods of treatment described herein can prevent or delay metastasis or recurrence of cancer.

In some embodiments, the subject is at risk, or genetically or otherwise predisposed (e.g., risk factor), to developing a cellular proliferative and/or differentiative disorder, e.g., a cancer, that has or has not been diagnosed.

As used herein, an “at risk” individual is an individual who is at risk of developing a condition to be treated, e.g., a cellular proliferative and/or differentiative disorder, e.g., a cancer. Generally, an “at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

### C. Lymphodepletion

In some embodiments, the patient is lymphodepleted before treatment.

Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 250 to about 500 mg/m² of cyclophosphamide, e.g., from or from about 250 to or to about 500, 250, 400, 500, about 250, about 400, or about 500 mg/m² of cyclophosphamide.

In some embodiments, lymphodepletion comprises administration of or of about 20 mg/m²/day to or to about 40 mg/m²/day fludarabine, e.g., 30 or about 30 mg/m²/day.

In some embodiments, lymphodepletion comprises administration of both cyclophosmamide and fludarabine.

In some embodiments, the patient is lymphodepleted by intravenous administration of cyclophosphamide (250 mg/m²/day) and fludarabine (30 mg/m²/day).

In some embodiments, the patient is lymphodepleted by intravenous administration of cyclophosphamide (500 mg/m²/day) and fludarabine (30 mg/m²/day).

In some embodiments, the lymphodepletion occurs no more than 5 days prior to the first dose of NK cells. In some embodiments, the lymphodepletion occurs no more than 7 days prior to the first dose of NK cells.

In some embodiments, lymphodepletion occurs daily for 3 consecutive days, starting 5 days before the first dose of NK cells (i.e., from Day −5 through Day −3).

In some embodiments, the lymphodepletion occurs on day −5, day −4 and day −3.

### D. Administration

### 1. NK Cells

In some embodiments, the NK cells, e.g., the NK cells described herein, e.g., the CAR-NK cells described herein are administered as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells drawn from a common batch or donor.

In some embodiments, the NK cells, e.g., the NK cells described herein, e.g., the CAR-NK cells described herein, are administered at or at about 1×10⁶ to or to about 1×10⁹ NK cells per dose. In some embodiments, the NK cells are administered at or at about 1×10⁶, at or at about 1×10⁷, at or at about 3×10⁷, at or at about 1×10⁸, at or at about 3×10⁸, or at or at about 1×10⁹ cells per dose.

In some embodiments, the NK cells are administered weekly. In some embodiments, the NK cells are administered for or for about 8 weeks.

In some embodiments, the NK cells are administered between one and four times over the course of nine months.

In some embodiments, the NK cells are cryopreserved in an infusion-ready media, e.g., a cryopreservation composition suitable for intravenous administration, e.g., as described herein.

In some embodiments, the NK cells are cryopreserved in vials containing from or from about 1×10⁷ to or to about 1×10⁹ cells per vial. In some embodiments, the NK cells are cryopreserved in vials containing a single dose.

In some embodiments, the cells are thawed, e.g., in a 37° C. water bath, prior to administration.

In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity.

In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing.

In some embodiments, the pharmaceutical composition is administered intravenously via syringe.

In some embodiments, 1 mL, 4 mL, or 10 mL of drug product is administered to the patient intravenously via syringe.

### 2. Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition.

In some embodiments, the cytokine is IL-2.

In some embodiments, the IL-2 is administered subcutaneously.

In some embodiments, the IL-2 is administered from between 1 to 4 or about 1 to about 4 hours following the conclusion of NK cell administration. In some embodiments, the IL-2 is administered at least 1 hour following the conclusion of NK cell administration. In some embodiments, the IL-2 is administered no more than 4 hours following the conclusion of NK cell administration. In some embodiments, the IL-2 is administered at least 1 hour after and no more than 4 hours following the conclusion of NK cell administration.

In some embodiments, the IL-2 is administered at up to 10 million IU/M², e.g., up to 1 million, 2 million, 3 million, 4 million, 5 million, 6 million, 7 million, 8 million, 9 million, or 10 million IU/m².

In some embodiments, the IL-2 is administered at or at about 1 million, at or at about 2 million, at or at about 3 million, at or at about 4 million, at or at about 5 million, at or at about 6 million, at or at about 7 million, at or at about 8 million, at or at about 9 million, at or at about 10 million IU/M²

In some embodiments, the IL-2 is administered at or at about 1×10⁶ IU/M². In some embodiments, the IL-2 is administered at or at about 2×10⁶ IU/M².

In some embodiments, less than 1×10⁶ IU/M² IL-2 is administered to the patient.

In some embodiments, a flat dose of IL-2 is administered to the patient. In some embodiments, a flat dose of 6 million IU or about 6 million IU is administered to the patient.

In some embodiments, IL-2 is not administered to the patient.

### E. Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### F. Combination Therapies

FOR CAR APPS INCLUDE THIS PARA: In some embodiments, the method comprises administering the NK cells described herein, e.g., the CAR-NK cells described herein, in combination with another therapy, e.g., an antibody, an NK cell engager, an antibody drug conjugate (ADC), a chemotherapy drug, e.g., a small molecule drug, an immune checkpoint inhibitor, and combinations thereof.

### 1. Antibodies

In some embodiments, the other therapy is an antibody.

In some embodiments, the antibody binds to a target selected from the group consisting of CD20, HER-2, EGFR, CD38, SLAMF7, GD2, ALK1, AMHR2, CCR2, CD137, CD19, CD26, CD32b, CD33, CD37, CD70, CD73, CD74, CD248, CLDN6, Clever-1, c-MET, CSF-1R, CXCR4, DKK1, DR5, Epha3, FGFR2b, FGFR3, FLT3, FOLR1, Globo-H, Glypican3, GM1, Grp78, HER-3, HGF, IGF-1R, IL1RAP, IL-8R, ILT4, Integrin alpha V, M-CSF, Mesothelin, MIF, MUC1, MUC16, MUC5AC, Myostatin, NKG2A, NOTCH, NOTCH2/3, PIGF, PRL3, PSMA, ROR1, SEMA4D, Sialyl Lewis A, Siglec15, TGF-b, TNFR3, TRAIL-R2, VEGF, VEGFR1, VEGFR2, Vimentin, and combinations thereof.

Suitable antibodies include, but are not limited to those shown in Table 3.

TABLE 3
Antibodies for Combination Therapy

| Target | Drug Name | Brand Name | Indication(s) | Reference |
|---|---|---|---|---|
| CD20 | Rituxan | Rituximab | DLBCL/FL, | Du et al., Auto Immun Highlights |
| | | | NHL, CLL, | (2017) 8(1): 12 |
| | | | RA, GPA, | |
| | | | MPA | |
| CD20 | Gazyva | Obinutuzumab | CLL, FL | Gagez et al., Curr Opin Oncol. 2014 |
| | | | | September; 26(5): 484-91 |
| CD20 | Arzerra | Ofatumumab | CLL | Robak, Curr Opin Mol Ther. 2008 |
| | | | | June; 10(3): 294-309 |
| CD20 | Ocrevus | Ocrelizumab | RMS, PPMS | Genovese et al., Arthritis Rheum. 2008 |
| | | | | September; 58(9): 2652-61 |
| CD20 | Zevalin | Ibritumomab | NHL | Wiseman et al., Eur J Nucl Med. 2000 |
| | | | | July; 27(7): 766-77 |
| CD20 | | Veltuzumab | NHL, CLL | Kalaycio et al. Leuk Lymphoma. |
| | | | | 2016; 57(4): 803-11 |
| CD20 | Bexxar | Tositumomab and | NHL | Vose et al., J Clin Oncol. 2000 |
| | | Iodine I 131 | | March; 18(6): 1316-23 |
| | | tositumomab | | |
| CD20 | | Ublituximab | NHL, CLL, | Sawas et al., Br J Haematol. 2017 |
| | | | RMS | April; 177(2): 243-253 |
| HER-2 | Herceptin | Trastuzumab | Breast, | Goldenberg, Clin Ther. 1999 |
| | | | Gastric | February; 21(2): 309-18 |
| HER-2 | Perjeta | Pertuzumab | Breast | Agus et al., J Clin Oncol. 2005 Apr. |
| | | | | 10; 23(11): 2534-43 |
| HER-2 | Margenza | Margetuximab | Breast | Bang et al., Ann Oncol. 2017 Apr. |
| | | | | 1; 28(4): 855-861 |
| EGFR | Erbitux | Cetuximab | CRC, HNC | Jonker et al., N Engl J Med 2007; |
| | | | | 357: 2040-2048 |
| EGFR | Vectibix | Panitumumab | CRC | Gibson et al., Clin Colorectal Cancer. |
| | | | | 2006 May; 6(1): 29-31 |
| EGFR | Portrazza | Necitumumab | NSCLC | Kuenen et al., Clin Cancer Res. 2010 |
| | | | | Mar. 15; 16(6): 1915-23 |
| CD38 | Darzalex | Daratumumab | MM | de Weers et al., J Immunol. 2011 Feb. |
| | | | | 1; 186(3): 1840-8 |
| CD38 | Sarclisa | Isatuximab | MM | Martin et al., Blood Cancer J. 2019 |
| | | | | Mar. 29; 9(4): 41 |
| SLAMF7 | Empliciti | Elotuzumab | MM | Lonial et al., N Engl J Med 2015; |
| | | | | 373: 621-631 |
| GD2 | Unituxin | Dinutuximab | NB | Hoy, Target Oncol. 2016 |
| | | | | April; 11(2):247-53 |
| GD2 | Danyelza | Naxitamab | NB | Markham, Drugs. 2021 February; 81(2): |
| | | | | 291-296 |
| ALK1 | PF- | Ascrinvacumab | Liver cancer | Simonelli et al., Ann Oncol. 2016 |
| | 03446962 | | | September; 27(9): 1782-7 |
| AMHR2 | GM-102 | Murlentamab | Ovarian | Leary et al., J Clin Oncol. 2019 |
| | | | Cancer | 37: 15_suppl, 2521-2521 |
| CCR2 | TAK-202 | Plozalizumab | Atherosclerosis, | Gilbert et al., Am J Cardiol. 2011 Mar. |
| | | | Melanoma | 15; 107(6): 906-11 |
| CD137 | BMS- | Urelumab | Melanoma, | Segal et al., Clin Cancer Res. 2017 |
| | 663513 | | Myeloma, | Apr. 15; 23(8): 1929-1936 |
| | | | NSCLC | |
| CD137 | PF- | Utomilumab | Ovarian | Segal et al., Clin Cancer Res. 2018 |
| | 05082566 | | Cancer | Apr. 15; 24(8): 1816-1823 |
| CD19 | AMG103 | Blinatumomab | ALL, NHL | Nadafi et al., Int J Mol Cell Med |
| | | | | (2015) 4(3): 143-151 |
| CD19 | SAR3419 | Coltuximab | ALL, NHL | Nadafi et al. |
| | | Ravtansine | | |
| CD19 | XmAb 5574 | MOR208 | ALL, NHL, | Nadafi et al. |
| | | | CLL | |
| CD19 | MEDI-551 | MEDI-551 | B-cell | Nadafi et al. |
| | | | malignancies, | |
| | | | CLL, | |
| | | | Multiple | |
| | | | Myeloma, | |
| | | | Scleroderma | |
| CD19 | SGN-19A | Denintuzumab | NHL | Nadafi et al. |
| | | Mafodotin | | |
| CD19 | | DI-B4 | B-cell | Nadafi et al. |
| | | | malignancies | |
| CD19 | Taplitumom | Taplitumomabpapt | B-cell | Nadafi et al. |
| | abpaptox | OX | malignancies | |
| CD19 | XmAb 5871 | XmAb 5871 | Autoimmune | Nadafi et al. |
| | | | Diseases | |
| CD19 | MDX-1342 | MDX-1342 | CLL, | Nadafi et al. |
| | | | Rheumatoid | |
| | | | Arthritis | |
| CD19 | AFM11 | AFM11 | NHL | Nadafi et al. |
| CD19 | ADCT-402 | Loncastuximab | ALL, NHL | Yu et al., Journal of Hematology & |
| | | Tesirine | | Oncology (2019) 12(94) |
| CD19 | Monjuvi | Tafasitamab | NHL | Hoy, Drugs. 2020 November; 80(16): |
| | | | (DLBCL) | 1731-1737 |
| CD26 | Begedina | Begelomab | Graft versus | Bacigalupo et al., Bone Marrow |
| | | | host disease | Transplant. 2020 August; 55(8): |
| | | | | 1580-1587 |
| CD32b | BI-1206 | BI-1206 | BCL, CLL | Trial ID: NCT04219254 |
| CD33 | Mylotarg | Gemtuzumab | AML | Stasi, Expert Opin Biol Ther. 2008 |
| | | Ozogamicin | | April; 8(4): 527-40 |
| CD33 | SGN-33 | Lintuzumab | AML | Trial ID: NCT02998047 |
| CD37 | BI 836826 | BI 836826 | DLBCL, | Trial ID: NCT02538614 |
| | | | CLL, NHL | |
| CD37 | IMGN529 | Naratuximab | DLBCL, | Yu et al., Journal of Hematology & |
| | | emtansine | NHL | Oncology (2019) 12(94) |
| CD37 | AGS67E | AGS67E | DLBCL, | Yu et al. |
| | | | NHL | |
| CD70 | BMS- | MDX-1203 | DLBCL, | Yu et al. |
| | 936561 | | MCL | |
| CD70 | SGN-75 | Vorsetuzumab | NHL | Yu et al. |
| | | mafodotin | | |
| CD73 | MEDI9447 | Oleclumab | Pancreatic | Geoghegan et al., MAbs. |
| | | | cancer | 2016; 8(3): 454-67 |
| CD73 | AK119 | AK119 | Covid-19, | Trial ID: NCT04516564 |
| | | | Solid Tumors | |
| CD74 | hLL1-DOX | Milatuzumab | MM | Yu et al. |
| | | doxorubicin | | |
| CD74 | STRO-001 | STRO-001 | MM, NHL | Trial ID: NCT03424603 |
| CD248 | Ontecizumab | Ontuxizumab | MM, Soft | D'Angelo et al., Invest New Drugs. |
| | | | tissue sarcoma | 2018 February; 36(1): 103-113 |
| CLDN6 | IMAB027 | ASP1650 | Testicular | Trial ID: NCT03760081 |
| | | | cancer | |
| Clever-1 | Clevegen | Bexmarilimab | Solid tumors | Trial ID: NCT03733990 |
| c-MET | MetMAb | Onartuzumab | NSCLC | Hughes et al., Trends Cancer (2018) |
| | | | | 4(2): 94-97 |
| c-MET | AMG-102 | Rilotumumab | Gastric | Waddell et al., Immunotherapy. |
| | | | cancer | 2014; 6(12): 1243-53 |
| CSF-1R | FPA-008 | Cabiralizumab | MM, NSCLC | Trial ID: NCT04050462 |
| CSF-1R | RG-7155 | Emactuzumab | Ovarian | Trial ID: NCT03708224 |
| | | | cancer | |
| CSF-1R | IMC CS4 | LY3022855 | MM | Trial ID: NCT03153410 |
| CSF-1R | AMB 051 | AMG 820 | Solid tumors | Trial ID: NCT04731675 |
| CSF-1R | SNDX-6352 | Axatilimab | Graft versus | Trial ID: NCT04710576 |
| | | | host disease | |
| CXCR4 | BMS- | Ulocuplumab | Leukemia | Bobkov et al., Mol Pharmacol (2019) |
| | 936564 | | | 96: 753-764 |
| CXCR4 | LY2624587 | LY2624587 | Metastatic | Bobkov et al. |
| | | | Cancer | |
| CXCR4 | PF- | PF-06747143 | AML | Bobkov et al. |
| | 06747143 | | | |
| CXCR4 | F50067 | hz515H7 | MM | Bobkov et al. |
| CXCR4 | MEDI3185 | MEDI3185 | Hematologic | Bobkov et al. |
| | | | malignancies | |
| DKK1 | DKN-01 | DKN-01 | Gastric | Wall et al., Expert Opin Investig |
| | | | cancer | Drugs. 2020 July; 29(7): 639-644 |
| DKK1 | BHQ880 | BHQ880 | MM | Fulciniti et al., Blood. 2009 Jul. |
| | | | | 9; 114(2):371-9 |
| DR5 | AD5-10 | Zaptuzumab | Solid tumors | Zhang et al., Theranostics. 2019 Jul. |
| | | | | 13; 9(18): 5412-5423 |
| DR5 | AMG655 | Conatumumab | Colon, | Rosevear et al., Curr Opin Investig |
| | | | pancreatic | Drugs. 2010 June; 11(6): 688-98 |
| | | | cancer | |
| DR5 | PRO955780 | Drozitumab | NHL, | Kang et al., Clin Cancer Res. 2011 |
| | | | NSCLC | May 15; 17(10): 3181-92 |
| DR5 | ETR2-ST01 | Lexatumumab | Solid tumors | Plummer et al., Clin Cancer Res. 2007 |
| | | | | Oct. 15; 13(20): 6187-94 |
| DR5 | CS-1008 | Tigatuzumab | Solid tumors | Reck et al., Lung Cancer. 2013 |
| | | | | December; 82(3): 441-8 |
| DR5 | | DS-8273a | Solid tumors | Forero et al., Invest New Drugs. 2017 |
| | | | | June; 35(3): 298-306 |
| Epha3 | KB004 | KB004 | Glioblastoma | Swords et al., Leuk Res. 2016 |
| | | | | November; 50: 123-131 |
| FGFR2b | FPA-144 | Bemarituzumab | Gastric | Catenacci et al., J Clin Oncol. 2020 |
| | | | cancer | Jul. 20; 38(21): 2418-2426 |
| FGFR2b | BAY | Aprutumab | Solid tumors | Kim et al., Target Oncol. 2019 |
| | 1187982 | ixadotin | | October; 14(5): 591-601 |
| FGFR2b | BAY- | Aprutumab | Solid tumors | Trial ID: NCT01881217 |
| | 1179470 | | | |
| FGFR3 | LY3076226 | LY3076226 | Solid tumors | Trial ID: NCT02529553 |
| FLT3 | IMC-EB10 | IMC-EB10 | AML | Piloto et al., Cancer Res. 2006 May |
| | | | | 1; 66(9): 4843-51 |
| | AGS 62P1 | ASP1235 | AML | Trial ID: NCT02864290 |
| FOLRI | MORAb- | Farletuzumab | Ovarian | Sato et al., Onco Targets Ther. 2016 |
| | 003 | | cancer | Mar. 7; 9: 1181-8 |
| Globo-H | OBI-833 | OBI-833 | Solid tumors | Trial ID: NCT02310464 |
| Globo-H | OBI-888 | OBI-888 | Solid tumors | Trial ID: NCT03573544 |
| Globo-H | OBI-999 | OBI-999 | Solid tumors | Trial ID: NCT04084366 |
| Glypican3 | GC33 | Codrituzumab | Liver cancer | Abou-Alfa et al., J Hepatol. 2016 |
| | | | | August; 65(2): 289-95 |
| Glypican3 | | ERY974 | Solid tumors | Ishiguro et al., Sci Transl Med. 2017 |
| | | | | Oct. 4; 9(410) |
| GM1 | BMS986012 | BMS-986012 | Lung cancer | Ponath et al., Clin Cancer Res. 2018 |
| | | | | Oct. 15; 24(20): 5178-5189 |
| Grp78 | PAT-SM6 | PAT-SM6 | Multiple | Hensel et al., Melanoma Res. 2013 |
| | | | myeloma | August; 23(4): 264-75 |
| HER-3 | U3-1402 | Patritumab | NSCLC, | Hashimoto et al., Clin Cancer Res. |
| | | deruxtecan | Solid tumors | 2019 Dec. 1; 25(23): 7151-7161 |
| HGF | AMG-102 | Rilotumumab | Solid tumors | Waddell et al., Immunotherapy. |
| | | | | 2014; 6(12): 1243-53 |
| HGF | AV-299 | Ficlatuzumab | AML, | Bauman et al., Cancers (Basel). 2020 |
| | | | NSCLC | June 11; 12(6): 1537 |
| HGF | L2G7 | TAK-701 | Solid tumors | Okamoto et al., Mol Cancer Ther. |
| | | | | 2010 October; 9(10): 2785-92 |
| IGF-1R | IMC-A12 | Cixutumumab | EWS, HCC | Chen et al., Chin J Cancer (2013) |
| | | | | 32(5): 242-252 |
| IGF-1R | CP-751 | Figitumumab | EWS, ACC | Chen et al. |
| IGF-1R | MK-0646 | Dalotuzumab | Colorectal | Chen et al. |
| | | | cancer | |
| IGF-1R | AMG 479 | Ganitumab | EWS, DRCT | Chen et al. |
| IGF-1R | | R1507 | EWS | Chen et al. |
| IGF-1R | AVE-1642 | VRDN 001 | MM, Breast | Trial ID: NCT01233895 |
| | | | cancer | |
| IL1RAP | CAN04 | Nidanilimab | NSCLC | Awada et al., J Clin Oncol. 2019 May; |
| | | | | 37: 2504-2504 |
| IL-8R | BMS- | HuMax-IL8 | Covid-19, | Bilusic et al., J Immunother Cancer. |
| | 986253 | | NSCLC | 2019 Sep. 5; 7(1): 240 |
| ILT4 | JTX-8064 | JTX-8064 | Solid tumors | Trial ID: NCT04669899 |
| Integrin | IMGN388 | IMGN388 | Solid tumors | Trial ID: NCT00721669 |
| alpha V | | | | |
| Integrin | CNTO-95 | Intetumumab | MM | O'Day et al., Br J Cancer. 2011 Jul. |
| alpha V | | | | 26; 105(3): 346-52 |
| Integrin | EMD52579 | Abituzumab | Colorectal | Jiang et al., Mol Cancer Res. 2017 |
| alpha V | 7 | | cancer | July; 15(7): 875-883 |
| Integrin | MEDI-522 | Etaracizumab | MM, | Hersey et al., Cancer. 2010 Mar. |
| alpha V | | | Colorectal | 15; 116(6): 1526-34 |
| | | | cancer | |
| Integrin | VPI-2690B | VPI-2690B | Diabetic | Trial ID: NCT02251067 |
| alpha V | | | nephropathies | |
| M-CSF | MCS-110 | Lacnotuzumab | Breast | Pognan et al., J Pharmacol Exp Ther. |
| | | | cancer, | 2019 June; 369(3): 428-442 |
| | | | Gastric | |
| | | | cancer | |
| Mesothelin | MORAb- | amatuximab | Mesothelioma | Baldo et al., Onco Targets Ther. 2017 |
| | 009 | | | Nov. 8; 10: 5337-5353 |
| Mesothelin | SS1(dsFv)- | SS1P | Neoplasms | Hassan et al., J Clin Oncol. 2016 |
| | PE38 | | | December; 34(34): 4171-4179 |
| Mesothelin | BAY 94- | Anetumab | Mesothelioma | Hassan et al., J Clin Oncol. 2020 Jun. |
| | 9343 | ravtansine | | 1; 38(16): 1824-1835 |
| Mesothelin | RG7600 | DMOT4039A | Pancreatic | Hassan et al., J Clin Oncol. 2016 |
| | | | cancer, | December; 34(34): 4171-4179 |
| | | | ovarian | |
| | | | cancer | |
| Mesothelin | BMS- | BMS-986148 | Solid Tumors | Hassan et al., J Clin Oncol. 2016 |
| | 986148 | | | December; 34(34): 4171-4179 |
| MIF | BAX69 | Imalumab | Colorectal | Mahalingham et al., Br J Clin |
| | | | cancer | Pharmacol. 2020 September; 86(9): |
| | | | | 1836-1848 |
| MUC1 | huC242- | Cantuzumab | Pancreatic | Tolcher et al., J Clin Oncol. 2003 Jan. |
| | DM1 | mertansine | cancer | 15; 21(2): 211-22 |
| MUC1 | hPAM4 | Clivatuzumab | Pancreatic | Liu et al., Oncotarget. 2015 Feb. |
| | | | cancer | 28; 6(6): 4274-85 |
| MUC1 | GT-MAB | Gatipotuzumab | Ovarian | Heublin et al., Int J Mol Sci. 2019 Jan. |
| | 2.5-GEXTM | | cancer | 12; 20(2): 295 |
| MUC1 | mAb- | AR20.5 | Pancreatic | de Bono et al., Ann Oncol. 2004 |
| | AR20.5 | | cancer | December; 15(12): 1825-33 |
| MUC16 | ACA 125 | Abagovomab | Ovarian | Sabbatini et al., J Clin Oncol. 2013 |
| | | | cancer | Apr. 20; 31(12): 1554-61 |
| MUC16 | DMUC5754 | Sofituzumab | Ovarian | Liu et al., Ann Oncol. 2016 |
| | A | vedotin | cancer | November; 27(11): 2124-2130 |
| MUC16 | DMUC4064 | THIOMABTM | Ovarian | Trial ID: NCT02146313 |
| | A | | cancer | |
| MUC5AC | PAM4 | Clivatuzumab | PDAC | Gold et al., Molecular Cancer (2013) |
| | | | | 12: 143 |
| MUC5AC | NPC-1C | Ensituximab | Pancreatic | Kim et al., Clin Cancer Res. 2020 Jul. |
| | | | cancer | 15; 26(14): 3557-3564 |
| Myostatin | MYO-029 | Stamulumab | Muscular | Trial ID: NCT00563810 |
| | | | atrophy, | |
| | | | Muscular | |
| | | | dystrophies | |
| Myostatin | PF- | Domagrozumab | Duchenne | Wagner et al., Neuromuscul Disord. |
| | 06252616 | | muscular | 2020 June; 30(6): 492-502 |
| | | | dystrophy | |
| Myostatin | LY- | Landogrozumab | Muscular | Golan et al., J Cachexia Sarcopenia |
| | 2495655 | | atrophy, | Muscle. 2018 October; 9(5): 871-879 |
| | | | Pancreatic | |
| | | | cancer | |
| Myostatin | REGN-1033 | Trevogrumab | Muscular | Trial ID: NCT01720576 |
| | | | atrophy | |
| Myostatin | SRK-015 | Apitegromab | Spinal | Trial ID: NCT03921528 |
| | | | muscular | |
| | | | atrophy | |
| NKG2A | IPH2201 | Monalizumab | Breast | Andre et al., Cell. 2018 Dec. |
| | | | cancer; | 13; 175(7): 1731-1743 |
| | | | NSCLC | |
| NOTCH | OMP- | Demcizumab | NSCLC | Takebe et al., Pharmacol Ther (2014) |
| | 21M18 | | | 141(2): 140-149 |
| NOTCH | REGN421/S | Enoticumab | NSCLC, | Takebe et al. |
| | AR153192 | | Ovarian | |
| | | | cancer | |
| NOTCH | OPM- | Brontictuzumab | Solid tumors | Takebe et al. |
| | 52M51 | | | |
| NOTCH | OMP-59R5 | Tarextumab | Sarcomas, | Takebe et al. |
| 2/3 | | | Rectal cancer | |
| PIGF | RO5323441 | TB-403 | Solid tumors | Martinsson-Niskanen et al., Clin Ther. |
| | | | | 2011 September; 33(9): 1142-9 |
| PRL3 | PRL3- | PRL3-zumab | Solid tumors | Trial ID: NCT04452955 |
| | ZUMAB | | | |
| PSMA | Capromab | Capromab | Prostate | Trial ID: NCT00992745 |
| | | pendetide | cancer | |
| PSMA | MT112 | Pasotuxizumab | Prostate | Hummel et al., Immunotherapy. 2021 |
| | | | cancer | February; 13(2): 125-141 |
| PSMA | | MDX1201-A488 | Prostate | Trial ID: NCT02048150 |
| | | | cancer | |
| PSMA | APVO 414 | MOR209/ES414 | Prostate | Hernandez-Hoyos et al., Mol Cancer |
| | | | cancer | Ther. 2016 September; 15(9): 2155-65 |
| PSMA | ARX-517 | ARX517 | Prostate | Trial ID: NCT04662580 |
| | | | cancer | |
| PSMA | ADCT 401 | MEDI3726 | Prostate | Cho et al., Mol Cancer Ther. 2018 |
| | | | cancer | October; 17(10): 2176-2186 |
| PSMA | | JNJ-63898081 | Prostate | Trial ID: NCT03926013 |
| | | | cancer | |
| PSMA | PSMA TTC | BAY 2315497 | Prostate | Hammer et al., Clin Cancer Res. 2020 |
| | | | cancer | Apr. 15; 26(8): 1985-1996 |
| PSMA | | TLX592 | Prostate | Trial ID: NCT04726033 |
| | | | cancer | |
| PSMA | DOTA- | Rosopatamab | Prostate | Vallabhajosula et al., Curr |
| | HUJ-591 | tetraxetan | cancer | Radiopharm. 2016; 9(1): 44-53 |
| PSMA | | PSMA ADC | Prostate | Petrylak et al., Prostate. 2020 |
| | | | cancer | January; 80(1): 99-108 |
| ROR1 | UC-961 | Cirmtuzumab | CLL, MCL | Choi et al., Cell Stem Cell. 2018 Jun. |
| | | | | 1; 22(6): 951-959 |
| SEMA4D | VX15/2503 | Pepinemab | NSCLC, MM | |
| Sialyl | MVT-5873 | MVT-5873 | Colorectal | Gupta et al., J Gastrointest Oncol. |
| Lewis A | | | cancer | 2020 April; 11(2): 231-235 |
| Sialyl | AbGn-7 | AbGn-7 | Gastric | Trial ID: NCT01466569 |
| Lewis A | | | cancer | |
| Siglec 15 | NC318 | NC318 | Solid tumors | Trial ID: NCT03665285 |
| TGF-b | | SRK-181 | Solid tumors | Trial ID: NCT04291079 |
| TGF-b | M-7824 | Bintrafusp alfa | NSCLC, | Yoo et al., J Immunother Cancer. 2020 |
| | | | Solid tumors | May; 8(1): e000564 |
| TGF-b | GC-1008 | Fresolimumab | MM | Rice et al., J Clin Invest. 2015 Jul. |
| | | | | 1; 125(7): 2795-807 |
| TGF-b | | LY2382770 | Diabetic | Trial ID: NCT01113801 |
| | | | nephropathie | |
| | | | S | |
| TGF-b | NIS-793 | NIS793 | Pancreatic | Trial ID: NCT04390763: |
| | | | cancer | |
| TGF-b | | SAR439459 | Solid tumors | Trial ID: NCT03192345 |
| TGF-b | | Metelimumab | Cancer, | Lord et al., MAbs. 2018 |
| | | | Scleroderma | April; 10(3): 444-452 |
| TGF-b | IMC TR1 | LY3022859 | Solid tumors | Tolcher et al., Cancer Chemother |
| | | | | Pharmacol. 2017 April; 79(4): 673-680 |
| TNFR3 | Baminercept | BG9924 | Rheumatoid | Trial ID: NCT00664716 |
| | | | arthritis | |
| TRAIL- | CS-1008 | Tigatuzumab | Breast | Cheng et al., J Hepatol. 2015 |
| R2 | | | cancer, | October; 63(4): 896-904 |
| | | | NSCLC | |
| TRAIL- | AMG-655 | Conatumumab | Solid tumors | Bajaj et al., Expert Opin Biol Ther. |
| R2 | | | | 2011 November; 11(11): 1519-24 |
| TRAIL- | PRO-95780 | Drozitumab | NHL, | Lima et al., Cancer Invest. 2012 |
| R2 | | | NSCLC | December; 30(10): 727-31 |
| TRAIL- | HGS-ETR2 | Lexatumumab | Solid tumors | Plummer et al., Clin Cancer Res. 2007 |
| R2 | | | | Oct. 15; 13(20): 6187-94 |
| TRAIL- | TAS-266 | TAS266 | Solid tumors | Trial ID: NCT01529307 |
| R2 | | | | |
| TRAIL- | GEN1029 | Benufutamab | Solid tumors | Overdijk et al., Mol Cancer Ther. 2020 |
| R2 | | | | October; 19(10): 2126-2138 |
| TRAIL- | RO- | RG7386 | Solid tumors | Brunker et al., Mol Cancer Ther. 2016 |
| R2 | 6874813 | | | May; 15(5): 946-57 |
| TRAIL- | JCT-205 | INBRX-109 | Solid tumors | Trial ID: NCT03715933 |
| R2 | | | | |
| VEGF | Avastin | Bevacizumab | NSCLC, MM | Garcia et al., Cancer Treat Rev. 2020 |
| | | | | June; 86: 102017 |
| VEGF | Lucentis | Ranibizumab | Macular | Gross et al., JAMA Ophthalmol. 2018 |
| | | | degeneration | Oct. 1; 136(10): 1138-1148 |
| VEGFR1 | IMC-18F1 | Icrucumab | Breast cancer | LoRusso et al., Invest New Drugs. |
| | | | | 2014 April; 32(2): 303-11 |
| VEGFR2 | Cyramza | Ramucirumab | NSCLC, | Khan et al., Expert Opin Biol Ther. |
| | | | Colorectal | 2019 November; 19(11): 1135-1141 |
| | | | cancer | |
| VEGFR2 | Tanibiruma | Olinvacimab | Glioblastoma | Lee et al., Drug Des Devel Ther. 2018 |
| | b | | | Mar. 8; 12: 495-504 |
| VEGFR2 | | Gentuximab | Solid tumors | Chamie et al., JAMA Oncol. 2017 Jul. |
| | | | | 1; 3(7): 913-920 |
| VEGFR2 | CDP-791 | Alacizumab pegol | NSCLC | Trial ID: NCT00152477 |
| VEGFR2 | HLX-06 | Vulinacimab | Solid tumors | Trial ID: NCT03494231 |
| VEGFR2 | | MSB0254 | Solid tumors | Trial ID: NCT04381325 |
| VEGFR2 | | AK109 | Solid tumors | Trial ID: NCT04547205 |
| Vimentin | CLNH11 | Pritumumab | Glioma | Babic et al., Hum Antibodies. 2018 |
| | | | | Feb. 5; 26(2): 95-101 |
| Vimentin | | 86C | Glioblastoma | Stouhalova et al., Cancers (2020) |
| | | | | 12(1): 184 |

### 2. Small Molecule/Chemotherapy Drugs

In some embodiments, the additional therapy is a small molecule drug. In some embodiments, the additional therapy is a chemotherapy drug. In some embodiments, the additional therapy is a small molecule chemotherapy drug. Such small molecule drugs can include existing standard-of-care treatment regimens to which adoptive NK cell therapy is added. In some cases, the use of the NK cells described herein can enhance the effects of small molecule drugs, including by enhancing the efficacy, reducing the amount of small molecule drug necessary to achieve a desired effect, or reducing the toxicity of the small molecule drug.

In some embodiments, the drug is selected from the group consisting of

In some embodiments, the drug is [(1S,2S,3R,4S,7R,9S,10S,12R,15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.0^{3,10}.0^{4,7}]heptadec-13-en-2-yl]benzoate (docetaxel) or a pharmaceutically acceptable salt thereof.

In some embodiments, the drug is [(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0^{4,7}]heptadec-13-en-2-yl]benzoate (paclitaxel) or a pharmaceutically acceptable salt thereof.

In some embodiments, the drug is 6-N-(4,4-dimethyl-5H-1,3-oxazol-2-yl)-4-N-[3-methyl-4-([1,2,4]triazolo[1,5-a]pyridin-7-yloxy)phenyl]quinazoline-4,6-diamine (tucatinib) or a pharmaceutically acceptable salt thereof.

In some embodiments, the drug is pentyl N-[1-[(2R,3R,4S,5R)-3,4-dihydroxy-5-methyloxolan-2-yl]-5-fluoro-2-oxopyrimidin-4-yl]carbamate (capecitabine) or a pharmaceutically acceptable salt thereof.

In some embodiments, the drug is azanide; cyclobutane-1,1-dicarboxylic acid; platinum (2+) (carboplatin) or a pharmaceutically acceptable salt thereof.

In some embodiments, the drug is methyl (1R,9R,10S,11R,12R,19R)-11-acetyloxy-12-ethyl-4-[(12S,14R)-16-ethyl-12-methoxycarbonyl-1,10-diazatetracyclo[12.3.1.0^{3,11}.0^{4,9}]octadeca-3(11),4,6,8,15-pentaen-12-yl]-10-hydroxy-5-methoxy-8-methyl-8,16-diazapentacyclo[10.6.1.0^{1,9}.0^{2,7}.0^{16,19}]nonadeca-2,4,6,13-thetraene-10-carboxylate (vinorelbine) or a pharmaceutically acceptable salt thereof.

In some embodiments, the drug is N-[3-chloro-4-[(3-fluorophenyl) methoxy]phenyl]-6-[5-[(2-methylsulfonylethylamino)methyl]furan-2-yl]quinazolin-4-amine (lapatinib) or a pharmaceutically acceptable salt thereof.

In some embodiments, the drug is (E)-N-[4-[3-chloro-4-(pyridin-2-ylmethoxy)anilino]-3-cyano-7-ethoxyquinolin-6-yl]-4-(dimethylamino)but-2-enamide (neratinib) or a pharmaceutically acceptable salt thereof.

In some embodiments, the drug is 6-acetyl-8-cyclopentyl-5-methyl-2-[(5-piperazin-1-ylpyridin-2-yl)amino]pyrido[2,3-d]pyrimidin-7-one (palbociclib) or a pharmaceutically acceptable salt thereof.

In some embodiments, the drug is 7-cyclopentyl-N,N-dimethyl-2-[(5-piperazin-1-ylpyridin-2-yl)amino]pyrrolo[2,3-d]pyrimidine-6-carboxamide (ribociclib) or a pharmaceutically acceptable salt thereof.

In some embodiments, the drug is N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (abemaciclib) or a pharmaceutically acceptable salt thereof.

In some embodiments, the drug is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone (everolimus) or a pharmaceutically acceptable salt thereof.

In some embodiments, the drug is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib) or a pharmaceutically acceptable salt thereof.

In some embodiments, the drug is 4-[[3-[4-(cyclopropanecarbonyl)piperazine-1-carbonyl]-4-fluorophenyl]methyl]-2H-phthalazin-1-one (olaparib) or a pharmaceutically acceptable salt thereof.

In some embodiments, the drug is (11S,12R)-7-fluoro-11-(4-fluorophenyl)-12-(2-methyl-1,2,4-triazol-3-yl)-2,3,10-triazatricyclo[7.3.1.0^{5,13}]trideca-1,5(13),6,8-tetraen-4-one (talazoparib) or a pharmaceutically acceptable salt thereof.

In some embodiments, the drug is N-[2-[2-(dimethylamino)ethyl-methylamino]-4-methoxy-5-[[4-(1-methylindol-3-yl)pyrimidin-2-yl]amino]phenyl]prop-2-enamid (osimertinib) or a pharmaceutically acceptable salt thereof.

In some embodiments, the drug is N-(3-chloro-4-fluorophenyl)-7-methoxy-6-(3-morpholin-4-ylpropoxy) quinazolin-4-amine (gefitinib) or a pharmaceutically acceptable salt thereof.

In some embodiments, the drug is N-(3-ethynylphenyl)-6,7-bis(2-methoxyethoxy) quinazolin-4-amine (erlotinib) or a pharmaceutically acceptable salt thereof.

In some embodiments, the drug is (E)-N-[4-(3-chloro-4-fluoroanilino)-7-[(3S)-oxolan-3-yl]oxyquinazolin-6-yl]-4-(dimethylamino)but-2-enamide (afatinib) or a pharmaceutically acceptable salt thereof.

In some embodiments, the drug is azane; dichloroplatinum (cisplatin, platinol) or a pharmaceutically acceptable salt thereof.

In some embodiments, the drug is azanide; cyclobutane-1,1-dicarboxylic acid; platinum (2+) (carboplatin) or a pharmaceutically acceptable salt thereof

In some embodiments, the drug is 4-amino-1-[(2R,4R,5R)-3,3-difluoro-4-hydroxy-5-(hydroxymethyl)oxolan-2-yl]pyrimidin-2-one (gemcitabine) or a pharmaceutically acceptable salt thereof.

In some embodiments, the drug is (2S)-2-[[4-[2-(2-amino-4-oxo-3,7-dihydropyrrolo[2,3-d]pyrimidin-5-yl)ethyl]benzoyl]amino]pentanedioic acid (pemetrexed) or a pharmaceutically acceptable salt thereof.

In some embodiments, the drug is N,N-bis(2-chloroethyl)-2-oxo-1,3,2λ⁵-oxazaphosphinan-2-amine (cyclophosphamide) or a pharmaceutically acceptable salt thereof.

In some embodiments, the drug is (2R,3S,4S,5R)-2-(6-amino-2-fluoropurin-9-yl)-5-(hydroxymethyl)oxolane-3,4-diol (fludarabine) or a pharmaceutically acceptable salt thereof.

In some embodiments, the drug is (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (doxorubicin) or a pharmaceutically acceptable salt thereof.

In some embodiments, the drug is methyl (1R,9R,10S,11R,12R,19R)-11-acetyloxy-12-ethyl-4-[(13S,15S,17S)-17-ethyl-17-hydroxy-13-methoxycarbonyl-1,11-diazatetracyclo[13.3.1.0^{4,12}.0^{5,10}]nonadeca-4(12),5,7,9-tetraen-13-yl]-8-formyl-10-hydroxy-5-methoxy-8,16-diazapentacyclo[10.6.1.0^{1,9}.0^{2,7}.0^{16,19}]nonadeca-2,4,6,13-tetraene-10-carboxylate (vincristine) or a pharmaceutically acceptable salt thereof.

In some embodiments, the drug is (8S,9S,10R,13S,14S,17R)-17-hydroxy-17-(2-hydroxyacetyl)-10,13-dimethyl-6,7,8,9,12,14,15,16-octahydrocyclopenta[a]phenanthrene-3,11-dione (prednisone) or a pharmaceutically acceptable salt thereof.

In some embodiments, the drug is N,3-bis(2-chloroethyl)-2-oxo-1,3,2λ⁵-oxazaphosphinan-2-amine (ifosfamide) or a pharmaceutically acceptable salt thereof.

In some embodiments, the drug is (5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one (etopside) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (dexamethasone) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (cytarabine) or a pharmaceutically acceptable salt thereof.

### 3. NK Cell Engagers

In some embodiments, the additional therapy is an NK cell engager, e.g., a bispecific or trispecific antibody.

In some embodiments, the NK cell engager is a bispecific antibody against CD16 and a disease-associated antigen, e.g., cancer-associated antigen, e.g., an antigen of cancers described herein. In some embodiments, the NK cell engager is a trispecific antibody against CD16 and two disease-associated antigens, e.g., cancer-associated antigens, e.g., antigens of cancers described herein.

### 4. Checkpoint Inhibitors

In some embodiments, the additional therapy is an immune checkpoint inhibitor.

In some embodiments, the immune checkpoint inhibitor is selected from the group consisting of a PD-1 inhibitor, a PD-L1 inhibitor, a CTLA-4 inhibitor, and combinations thereof.

In some embodiments, the immune checkpoint inhibitor is selected from the group consisting of a PD-1 inhibitor, a PD-L1 inhibitor, a CTLA-4 inhibitor, a VISTA inhibitor, a BTLA inhibitor, a TIM-3 inhibitor, a KIR inhibitor, a LAG-3 inhibitor, a TIGIT inhibitor, a CD-96 inhibitor, a SIRPα inhibitor, and combinations thereof.

In some embodiments, the immune checkpoint inhibitor is selected from the group consisting of a PD-1 inhibitor, a PD-L1 inhibitor, a CTLA-4 inhibitor, a LAG-3 (CD223) inhibitor, a TIM-3 inhibitor, a B7-H3 inhibitor, a B7-H4 inhibitor, an A2aR inhibitor, a CD73 inhibitor, a NKG2A inhibitor, a PVRIG/PVRL2 inhibitor, a CEACAM1 inhibitor, a CEACAM 5 inhibitor, a CEACAM 6 inhibitor, a FAK inhibitor, a CCL2 inhibitor, a CCR2 inhibitor, a LIF inhibitor, a CD47 inhibitor, a SIRPα inhibitor, a CSF-1 inhibitor, an M-CSF inhibitor, a CSF-1R inhibitor, an IL-1 inhibitor, an IL-1R3 inhibitor, an IL-RAP inhibitor, an IL-8 inhibitor, a SEMA4D inhibitor, an Ang-2 inhibitor, a CELVER-1 inhibitor, an Axl inhibitor, a phsphatidylserine inhibitor, and combinations thereof.

In some embodiments, the immune checkpoint inhibitor is selected from those shown in Table 4, or combinations thereof.

TABLE 4
Exemplary Immune Checkpoint Inhibitors

| Target | Inhibitor |
|---|---|
| LAG-3 (CD223) | LAG525 (IMP701), REGN3767 (R3767), BI 754,091, tebotelimab |
| | (MGD013), eftilagimod alpha (IMP321), FS118 |
| TIM-3 | MBG453, Sym023, TSR-022 |
| B7-H3, B7-H4 | MGC018. FPA150 |
| A2aR | EOS100850, AB928 |
| CD73 | CPI-006 |
| NKG2A | Monalizumab |
| PVRIG/PVRL2 | COM701 |
| CEACAMI | CM24 |
| CEACAM 5/6 | NEO-201 |
| FAK | Defactinib |
| CCL2/CCR2 | PF-04136309 |
| LIF | MSC-1 |
| CD47/SIRP | Hu5F9-G4 (SF9), ALX148, TTI-662, RRx-001 |
| CSF-1 | Lacnotuzumab (MCS110), LY3022855, SNDX-6352, emactuzumab |
| (M-CSF)/CSF-1R | (RG7155), pexidartinib (PLX3397) |
| IL-1 and IL-1R3 | CAN04, Canakinumab (ACZ885) |
| (IL-IRAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phosphatidylserine | Bavituximab |

In some embodiments, the immune checkpoint inhibitor is an antibody.

In some embodiments, the PD-1 inhibitor is selected from the group consisting of pembrolizumab, nivolumab, toripalimab, cemiplimab-rwlc, sintilimab, and combinations thereof.

In some embodiments, the PD-L1 inhibitor is selected from the group consisting of atezolizumab, durvalumab, avelumab, and combinations thereof.

In some embodiments, the CTLA-4 inhibitor is ipilimumab.

In some embodiments, the PD-1 inhibitor is selected from the group of inhibitors shown in Table 5.

TABLE 5
Exemplary PD-1 Inhibitor Antibodies

| Name | Internal Name | Antigen | Company |
|---|---|---|---|
| nivolumab | Opdivo, ONO-4538, | PD-1 | BMS, Medarex, Ono |
| | MDX-1106, BMS- | | |
| | 936558, 5C4 | | |
| pembrolizumab | Keytruda, MK-3475, | PD-1 | Merck (MSD), Schering- |
| | SCH 900475, | | Plough |
| | lambrolizumab | | |
| toripalimab | JS001, JS-001, | PD-1 | Junmeng Biosciences, Shanghai |
| | TAB001, | | Junshi, TopAlliance Bio |
| | Triprizumab | | |
| cemiplimab-rwlc | Libtayo, cemiplimab, | PD-1 | Regeneron, Sanofi |
| | REGN2810 | | |
| sintilimab | Tyvyt, IBI308 | PD-1 | Adimab, Innovent, Lilly |
| MEDI0680 | AMP-514 | PD-1 | Amplimmune, Medimmune |
| LZM009 | | PD-1 | Livzon |
| vudalimab | XmAb20717 | CTLA4, PD-1 | Xencor |
| SI-B003 | | CTLA4, PD-1 | Sichuan Baili |
| | | | Pharma, Systimmune |
| Sym021 | Symphogen patent | PD-1 | Symphogen |
| | anti-PD-1 | | |
| LVGN3616 | | PD-1 | Lyvgen Biopharma |
| MGD019 | | CTLA4, PD-1 | MacroGenics |
| MEDI5752 | | CTLA4, PD-1 | Medimmune |
| CS1003 | | PD-1 | CStone Pharma |
| IBI319 | IBI-319 | PD-1, Undisclosed | Innovent, Lilly |
| IBI315 | IBI-315 | HER2/neu, PD-1 | Beijing Hanmi, Innovent |
| budigalimab | ABBV-181, PR- | PD-1 | Abbvie |
| | 1648817 | | |
| Sunshine Guojian | 609A | PD-1 | Sunshine Guojian Pharma |
| patent anti-PD-1 | | | |
| F520 | | PD-1 | Shandong New Time Pharma |
| RO7247669 | | LAG-3, PD-1 | Roche |
| izuralimab | XmAb23104 | ICOS, PD-1 | Xencor |
| LY3434172 | | PD-1, PD-L1 | Lilly, Zymeworks |
| SG001 | | PD-1 | CSPC Pharma |
| QL1706 | PSB205 | CTLA4, PD-1 | Sound Biologics |
| AMG 404 | AMG404 | PD-1 | Amgen |
| MW11 | | PD-1 | Mabwell |
| GNR-051 | | PD-1 | IBC Generium |
| Ningbo Cancer Hosp. | HerinCAR-PD1 | PD-1 | Ningbo Cancer Hosp. |
| anti-PD-1 CAR | | | |
| Chinese PLA | | PD-1 | Chinese PLA Gen.Hosp. |
| Gen.Hosp. anti-PD-1 | | | |
| cetrelimab | JNJ-63723283 | PD-1 | Janssen Biotech |
| TY101 | | PD-1 | Tayu Huaxia |
| AK112 | | PD-1, VEGF | Akeso |
| EMB-02 | | LAG-3, PD-1 | EpimAb |
| pidilizumab | CT-011, hBat-1, | PD-1 | CureTech, Medivation, Teva |
| | MDV9300 | | |
| sasanlimab | PF-06801591, RN- | PD-1 | Pfizer |
| | 888 | | |
| balstilimab | AGEN2034, AGEN- | PD-1 | Agenus, Ludwig Inst., Sloan- |
| | 2034 | | Kettering |
| geptanolimab | CBT-501, GB226, | PD-1 | CBT Pharma, Genor |
| | GB 226, | | |
| | Genolimzumab, | | |
| | Genormab | | |
| RO7121661 | | PD-1, TIM-3 | Roche |
| AK104 | | CTLA4, PD-1 | Akeso |
| pimivalimab | JTX-4014 | PD-1 | Jounce |
| IBI318 | IBI-318 | PD-1, PD-L1 | Innovent, Lilly |
| BAT1306 | | PD-1 | Bio-Thera Solutions |
| ezabenlimab | BI754091, BI 754091 | PD-1 | Boehringer |
| Henan Cancer | Teripalimab | PD-1 | Henan Cancer Hospital |
| Hospital anti-PD-1 | | | |
| tebotelimab | | LAG-3, PD-1 | MacroGenics |
| sindelizumab | | PD-1 | Nanjing Medical U. |
| dostarlimab | ANB011, TSR-042, | PD-1 | AnaptysBio, Tesaro |
| | ABT1 | | |
| tislelizumab | BGB-A317 | PD-1 | BeiGene, Celgene |
| spartalizumab | PDR001, BAP049 | PD-1 | Dana-Farber, Novartis |
| retifanlimab | MGA012, | PD-1 | Incyte, MacroGenics |
| | INCMGA00012 | | |
| camrelizumab | SHR-1210 | PD-1 | Incyte, Jiangsu |
| | | | Hengrui, Shanghai Hengrui |
| zimberelimab | WBP3055, GLS-010, | PD-1 | Arcus, Guangzhou Gloria |
| | AB122 | | Bio, Harbin Gloria |
| | | | Pharma, WuXi Biologics |
| penpulimab | AK105 | PD-1 | Akeso, HanX Bio, Taizhou |
| | | | Hanzhong Bio |
| prolgolimab | BCD-100 | PD-1 | Biocad |
| HX008 | | PD-1 | Taizhou Hanzhong |
| | | | Bio, Taizhou HoudeAoke Bio |
| SCT-I10A | | PD-1 | Sinocelltech |
| serplulimab | HLX10 | PD-1 | Henlix |

In some embodiments, the PD-L1 inhibitor is selected from the group of inhibitors shown in Table 6.

TABLE 6
Exemplary PD-L1 Inhibitor Antibodies

| Name | Internal Name | Antigen | Company |
|---|---|---|---|
| durvalumab | Imfinzi, MEDI-4736, | PD-L1 | AstraZeneca, Celgene, Medimmune |
| | MEDI4736 | | |
| atezolizumab | Tecentriq, MPDL3280A, | PD-L1 | Genentech |
| | RG7446, YW243.55.S70, | | |
| | RO5541267 | | |
| avelumab | Bavencio, MSB0010718C, | PD-L1 | Merck Serono, Pfizer |
| | A09-246-2 | | |
| AMP-224 | | PD-L1 | Amplimmune, GSK, Medimmune |
| cosibelimab | CK-301, TG-1501 | PD-L1 | Checkpoint Therapeutics, Dana- |
| | | | Farber, Novartis, TG Therapeutics |
| lodapolimab | LY3300054 | PD-L1 | Lilly |
| MCLA-145 | | 4-1BB, PD-L1 | Merus |
| FS118 | | LAG-3, PD- | f-star, Merck Serono |
| | | L1 | |
| INBRX-105 | ES101 | 4-1BB, PD-L1 | Elpiscience, Inhibrx |
| Suzhou Nanomab | | PD-L1 | Suzhou Nanomab |
| patent anti-PD- | | | |
| L1 | | | |
| MSB2311 | | PD-L1 | Mabspace |
| BCD-13 | | PD-L1 | Biocad |
| opucolimab | HLX20, HLX09 | PD-L1 | Henlix |
| IBI322 | IBI-322 | CD47, PD-L1 | Innovent |
| LY3415244 | | PD-L1, TIM-3 | Lilly, Zymeworks |
| GR1405 | | PD-L1 | Genrix Biopharma |
| LY3434172 | | PD-1, PD-L1 | Lilly, Zymeworks |
| CDX-527 | | CD27, PD-L1 | Celldex |
| FS222 | | 4-1BB, PD-L1 | f-star |
| LDP | | PD-L1 | Dragonboat Biopharma |
| ABL503 | | 4-1BB, PD-L1 | ABL Bio |
| HB0025 | | PD-L1, VEGF | Huabo Biopharm |
| MDX-1105 | BMS-936559, 12A4 | PD-L1 | Medarex |
| garivulimab | BGB-A333 | PD-L1 | BeiGene |
| GEN1046 | | 4-1BB, PD-L1 | BioNTech, Genmab |
| NM21-1480 | | 4-1BB, PD- | Numab |
| | | L1, Serum | |
| | | Albumin | |
| bintrafusp alfa | M7824, MSB0011359C | PD- | Merck Serono, NCI |
| | | L1, TGFBRII | |
| pacmilimab | CX-072 | PD-L1 | CytomX |
| A167 | KL-A167 | PD-L1 | Harbour Biomed Ltd., Sichuan |
| | | | Kelun Pharma |
| IBI318 | IBI-318 | PD-1, PD-L1 | Innovent, Lilly |
| KN046 | | CTLA4, PD- | Alphamab |
| | | L1 | |
| STI-3031 | IMC-001 | PD-L1 | Sorrento |
| SHR-1701 | | PD-L1 | Jiangsu Hengrui |
| LP002 | | PD-L1 | Taizhou HoudeAoke Bio |
| STI-1014 | ZKAB001 | PD-L1 | Lee's Pharm, Sorrento |
| envafolimab | KN035 | PD-L1 | Alphamab |
| adebrelimab | SHR-1316 | PD-L1 | Jiangsu Hengrui, Shanghai Hengrui |
| CS1001 | | PD-L1 | CStone Pharma |
| TQB2450 | CBT-502 | PD-L1 | CBT Pharma, Chia Tai Tianqing |
| | | | Pharma |

In some embodiments, the CTLA-4 inhibitor is selected from the group of inhibitors shown in

TABLE 7
CTLA4 Inhibitor

| Name | Internal Name | Antigen | Company |
|---|---|---|---|
| ipilimumab | Yervoy, MDX-010, MDX101, | CTLA4 | Medarex |
| | 10D1, BMS-734016 | | |
| ATOR-1015 | ADC-1015 | CTLA4, OX40 | Alligator |
| vudalimab | XmAb20717 | CTLA4, PD-1 | Xencor |
| SI-B003 | | CTLA4, PD-1 | Sichuan Baili |
| | | | Pharma, Systimmune |
| MGD019 | | CTLA4, PD-1 | MacroGenics |
| MEDI5752 | | CTLA4, PD-1 | Medimmune |
| ADU-1604 | | CTLA4 | Aduro |
| BCD-145 | Q3W | CTLA4 | Biocad |
| CS1002 | | CTLA4 | CStone Pharma |
| REGN4659 | | CTLA4 | Regeneron |
| pavunalimab | XmAb22841 | CTLA4, LAG-3 | Xencor |
| AGEN1181 | | CTLA4 | Agenus |
| QL1706 | PSB205 | CTLA4, PD-1 | Sound Biologics |
| ADG126 | | CTLA4 | Adagene |
| KN044 | | CTLA4 | Changchun Intelli-Crown |
| ONC-392 | | CTLA4 | OncoImmune, Pfizer |
| BMS-986218 | | CTLA4 | BMS |
| BMS-986249 | | CTLA4 | BMS |
| BT-001 | TG6030 | CTLA4 | BioInvent |
| quavonlimab | MK-1308 | CTLA4 | Merck (MSD) |
| zalifrelimab | AGEN1884 | CTLA4 | Agenus, Ludwig Inst., Sloan- |
| | | | Kettering |
| AK104 | | CTLA4, PD-1 | Akeso |
| IBI310 | IBI-310 | CTLA4 | Innovent |
| KN046 | | CTLA4, PD-L1 | Alphamab |
| tremelimumab | ticilimumab, CP-675206, | CTLA4 | Amgen, Medimmune, Pfizer |
| | clone 11.2.1 | | |

In some embodiments, the immune checkpoint inhibitor is a small molecule drug. Small molecule checkpoint inhibitors are described, e.g., in WO2015/034820A1, WO2015/160641A2, WO2018/009505 A1, WO2017/066227 A1, WO2018/044963 A1, WO2018/026971 A1, WO2018/045142 A1, WO2018/005374 A1, WO2017/202275 A1, WO2017/202273 A1, WO2017/202276 A1, WO2018/006795 A1, WO2016/142852 A1, WO2016/142894 A1, WO2015/033301 A1, WO2015/033299 A1, WO2016/142886 A2, WO2016/142833 A1, WO2018/051255 A1, WO2018/051254 A1, WO2017/205464 A1, US2017/0107216 A1, WO2017/070089A1, WO2017/106634A1, US2017/0174679 A1, US2018/0057486 A1, WO2018/013789 A1, US2017/0362253 A1, WO2017/192961 A1, WO2017/118762 A1, US2014/199334 A1, WO2015/036927 A1, US2014/0294898 A1, US2016/0340391 A1, WO2016/039749 A1, WO2017/176608 A1, WO2016/077518 A1, WO2016/100608 A1, US2017/0252432 A1, WO2016/126646 A1, WO2015/044900 A1, US2015/0125491 A1, WO2015/033303 A1, WO2016/142835 A1, WO2019/008154 A1, WO2019/008152 A1, and WO2019023575A1.

In some embodiments, the PD-1 inhibitor is 2-[[4-amino-1-[5-(1-amino-2-hydroxypropyl)-1,3,4-oxadiazol-2-yl]-4-oxobutyl]carbamoylamino]-3-hydroxypropanoic acid (CA-170).

In some embodiments, the immune checkpoint inhibitor is (S)-1-(3-Bromo-4-((2-bromo-[1,1′-biphenyl]-3-yl)methoxy)benzyl)piperidine-2-carboxylic Acid.

In some embodiments, the immune checkpoint inhibitor is a peptide. See, e.g., Sasikumar et al., “Peptide and Peptide-Inspired Checkpoint Inhibitors: Protein Fragments to Cancer Immunotherapy,” Medicine in Drug Discovery 8:100073 (2020).

### V. VARIANTS

In some embodiments, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M.O., Ed, pp 353-358; BLAST program (Basic Local Alignment Search Tool; (Altschul, S. F., W. Gish, et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa.

As used herein, the term “cell culture medium” refers to a mixture for growth and proliferation of cells in vitro, which contains essential elements for growth and proliferation of cells such as sugars, amino acids, various nutrients, inorganic substances, etc.

A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### VII. EXAMPLES

The following examples are included for illustrative purposes only and are not intended to limit the scope of the invention.

### Example 1: CAR Costimulatory Structure Comprising OX40L

In some embodiments, the NK cells are CAR-NK cells. As shown in Error! Reference source not found., CAR-NKs comprising a co-stimulatory domain comprising OX40L exhibited greater cytotoxic potential than those without OX40L. In this example, the CAR-NK cells comprise an anti-HER2 scFv as described in US20200399397A1, which is hereby incorporated by reference in its entirety.

In vitro efficacy, proliferation, CAR expression, and in vitro efficacy was compared for NK-CARs comprising the CARs with anti-HER2 scFv with (SEQ ID NO: 64) and without OX40L (SEQ ID NO: 66) (Error! Reference source not found.). As shown in Error! Reference source not found. and Error! Reference source not found., both CAR-NK structures proliferated in tumor negative control cells and expressed the CAR. In vitro efficacy (CD107a expression, cytokine production, and percent lysis) is shown for various cell lines (HER2 positive and trastuzumab sensitive target cells (SKBR3, NCI-N87, and SKOV-3), HER2 positive and trastuzumab resistant target cells (HCC1954), and HER2 negative target cells (MDA-MB-468) in Error! Reference source not found., Error! Reference source not found., Error! Reference source not found., and Error! Reference source not found.). The OX40L containing CAR showed greater cytotoxic potential than that the CAR without OX40L against HER2 positive cell lines.

### Example 2: AB-201

A CAR-NK expressing the fusion protein having SEQ ID NO: 27 was produced by transducing NK cells with a vector comprising SEQ ID NO: 28.

### Example 3: CAR Constructs Expressing IL-15 have Increased Cytotoxicity

To investigate whether a CAR-NK which expresses both CAR and IL-15 has a synergistic effect on cytotoxicity, CAR-NK structures were generated as shown in Error! Reference source not found.

NK cells including NK, mock-NK, CAR-NK, CAR (t)-IL-15-NK, and CAR-IL-15-NK (AB-201) group were generated from cord blood of a healthy donor. The CD3 negative cells in cord blood unit were purified by using CD3+ cells positive isolation kit, and then they were used as seed cells.

The seed cells included CD56+ NK cells were stimulated with irradiated eHuT-78P cells and OKT3 and recombinant IL-2 (Proleukin) in complete serum-free medium (CellGro) on day 0.

The cultured NK cells were transduced by lentiviral vector on day 6 or 8 and were stimulated again with the irradiated eHuT-78P cells and OKT3 and IL-2 on day 14. At day 22, the cell groups were divided two groups again and cultured in the presence or absence of IL-2, respectively. Both transduced and non-transduced NK cells were cultured for 35 days in the presence of IL-2. As shown in Error! Reference source not found., IL-15 secreting transduced expressed the CAR stably until day 35. As shown in Error! Reference source not found., only IL-15 secreting transduced NK cells survived and expressed the CAR in the absence of IL-2. Moreover, the results show that expression of IL-15 increases the proportion of cells that are CAR+ in the absence of IL-2 (compare CAR-NK (43%) and CAR-IL-15-NK (91.3%) at day 29). As shown in Error! Reference source not found., NK cells not secreting IL-15 did not proliferate after day 22 and, as shown in Error! Reference source not found., their viability decreased rapidly after day 22. The results show that recombinant expression of IL-15 extends survival of NK cells even in the absence of IL-2.

To measure cytotoxicity, NK cells were cultured in the presence of IL-2 until day 22, and then cultured four more days in the absence of IL-2. At day 26, the NK cells were co-cultured with HCC1954 or SKOV3 at the E:T=0.3:1 ratio for long-term killing assay (Error! Reference source not found.) or E:T=1:1 ratio for IFNg ELISA (Error! Reference source not found.) for 6 days in the absence of IL-2. CAR-IL-15-NK cells had a higher cytotoxicity than that of other NK cells. These results show that the CAR comprising an OX40L costimulatory domain and IL-15 expression exhibited better and more sustained killing activity. The CAR-NK cells lacking IL-15 expression showed significantly reduced killing activity compared to the cells expressing IL-15 under these conditions. On day 32, the amount of IFNg in the culture supernatant was measured. The CAR-IL-15-NK cells produced the highest amount of IFNg, and it was correlated to the cytolytic activity results in Error! Reference source not found.

To measure IL-15 production, the non-transduced or transduced NK cells were cultured in the presence of IL-2 until day 28, and then cultured four more days in the absence of IL-2. At day 32, the indicated NK cells were co-cultured with HCC1954 or SKOV3 for 72 hours in the absence of IL-2, and the IL-15 levels in the cultured supernatant were determined by ELISA, as shown in Error! Reference source not found. These results show that co-culturing CAR-IL-15-NK cells in the presence of HER2+ target cells increased the amount of IL-15 produced by the NK cells. In contrast, the CAR (t)-IL-15-NK cells without costimulatory domains generated relatively constant amounts of basal IL-15 expression in both the absence of and presence of target cells. NK cells that lacked recombinant nucleic acids encoding IL-15 did not generate significant levels of IL-15 expression.

### Example 4: Secretion of IL-15 Maintains the Survival of Bystander NK Cells

NK cells and CAR-IL-15-NK (AB-201) cells were generated from two different donors. Cells were transduced to produce CAR-IL-15-NK. At day 14, the NK cells were re-stimulated and CAR-IL-15-NK cells were re-stimulated and sorted. At day 19, the NK cells were CFSE labeled and co-cultures were created by mixing CFSE NK cells and CAR-IL-15-NK cells at a 1:1 ratio. Cocultures either with or without IL-2 were carried out for 5 days. Fixable viability dye (Invitrogen #65-0865) was used to detect viable cells. As shown in Error! Reference source not found., despite the absence of IL-2, the frequency of living NK cells that co-cultured with CAR-IL-15-NK cells was not decreased in the experiments using two different donors.

### Example 5: Long Term Stability and Survival of CAR-NKs Expressing IL-15

NK cells expressing CARs with and without IL-15 (Error! Reference source not found.) were cultured as described in Example 4 to day 19. At day 19, they were cultured without IL-2. As shown in Error! Reference source not found., the CAR-expressing cells lacking IL-15 (3^{rd} CAR) had significantly reduced CAR expression levels (e.g., only 55.2% of max at day 30) as compared to cells expressing the CAR with IL-15 (4th CAR) (e.g., 97.1% of max at day 56). The CAR-expressing cells lacking IL-15 (3^{rd} CAR) also failed to persist as long, as none survived until day 44. As shown in Error! Reference source not found. and Error! Reference source not found., the cells expressing the CAR lacking IL-15 did not survive past day 37, whereas the cells expressing the CAR with IL-15 survived at least up to day 62, and also maintained viability. As shown in Error! Reference source not found., cells expressing IL-15 persisted better than cells lacking heterologous expression of IL-15 in the presence target cells.

### Example 6: AB-202 in Vitro Cytotoxicity

AB-202 displays significant cytotoxic activity against CD19+ target cells (Ramos). As shown in Error! Reference source not found., IFN-gamma secretion from AB-202 cells was increased when exposed to CD19+ Ramos tumor cells. As shown in Error! Reference source not found., AB-202 demonstrated greater activation and cytotoxic activity against CD19+ Ramos cell than non-CAR NK cells.

As shown in Error! Reference source not found., AB-202 displays significantly greater killing of CD19+ target cells than the CD-19 CAR-NKs based on Liu et al., “Use of CAR-Transduced Natural Killer Cells in CD19-Positive Lymphoid Tumors,” N Engl J Med 382:545-53 (2020). The AB-202 CAR (SEQ ID NO: 38) comprises an anti-CD19 scFv based on the FMC63 clone (SEQ ID NO: 37), an IgG1 hinge, an IgG1 spacer, a CD28 transmembrane domain, a CD28 costimulatory domain, an OX40L costimulatory domain, and a CD3ζ costimulatory domain. The NK cells expressing the AB-202 CAR were compared to NK cells expressing CD19-CAR CD28-CD3z-IL15 (SEQ ID NO: 39), which includes the same FMC63 clone (SEQ ID NO: 37), IgG1 hinge, IgG1 spacer, CD28 transmembrane domain, CD28 costimulatory domain, and CD3ζ costimulatory domain. Notably, the CD19-CAR CD28-CD3z-IL15 lacks the OX40L costimulatory domain.

The NK cells were incubated with Raji B-cell lymphoma cells at an E:T ratio of 0.3:1 for more than four days. The growth of Raji cells was monitored by measuring phase area confluence using Incucyte® live cell imaging of the cultures. Both AB-202 and the NK cells expressing CD19-CAR CD28-CD3z-IL15 inhibited Raji cell growth compared to Raji cells grown in the absence of NK cells (Error! Reference source not found.). AB-202 cells, however, inhibited significantly more growth and was more cytotoxic than NK cells expressing CD19-CAR CD28-CD3z-IL15. The results show that the use of a CAR costimulatory structure that includes the intracellular signaling portion of OX40L improves the cell killing activity of NK cells.

SEQUENCES

| SEQ ID NO: and | |
|---|---|
| DESCRIPTION | SEQUENCE |
| SEQ ID NO: 1 | MEYASDASLDPEAPWPPAPRARACRVLPWALVAGLLLLLLLAAACAVFLACPWAVSGA |
| Sequence of 4-1BBL | RASPGSAASPRLREGPELSPDDPAGLLDLRQGMFAQLVAQNVLLIDGPLSWYSDPGLA |
| that can be expressed | GVSLTGGLSYKEDTKELVVAKAGVYYVFFQLELRRVVAGEGSGSVSLALHLQPLRSAA |
| by feeder cells | GAAALALTVDLPPASSEARNSAFGFQGRLLHLSAGQRLGVHLHTEARARHAWQLTQGA |
| | TVLGLFRVTPEIPAGLPSPRSE |
| SEQ ID NO: 2 | MALPVTALLLPLALLLHAARPQDRHMIRMRQLIDIVDQLKNYVNDLVPEFLPAPEDVE |
| Sequence of a | TNCEWSAFSCFQKAQLKSANTGNNERIINVSIKKLKRKPPSTNAGRRQKHRLTCPSCD |
| membrane bound IL- | SYEKKPPKEFLERFKSLLQKMIHQHLSSRTHGSEDSAKPTTTPAPRPPTPAPTIASQP |
| 21(mbIL-21) that can | LSLRPEACRPAAGGAVHTRGLDFACDIYIWAPLAGTCGVLLLSLVITLY |
| be expressed by | |
| feeder cells | |
| SEQ ID NO: 3 | MSTESMIRDVELAEEALPKKTGGPQGSRRCLFLSLFSFLIVAGATTLFCLLHFGVIGP |
| Sequence of a mutated | QREEFPRDLSLISPLAQPVRSSSRTPSDKPVAHVVANPQAEGQLQWLNRRANALLANG |
| TNF alpha (mTNF-a) | VELRDNQLVVPSEGLYLIYSQVLFKGQGCPSTHVLLTHTISRIAVSYQTKVNLLSAIK |
| that can be expressed | SPCQRETPEGAEAKPWYEPIYLGGVFQLEKGDRLSAEINRPDYLDFAESGQVYFGIIA |
| by feeder cells | L |
| SEQ ID NO: 4 | MERVQPLEENVGNAARPRFERNKLLLVASVIQGLGLLLCFTYICLHFSALQVSHRYPR |
| Sequence of OX40L | IQSIKVQFTEYKKEKGFILTSQKEDEIMKVQNNSVIINCDGFYLISLKGYFSQEVNIS |
| that can be expressed | LHYQKDEEPLFQLKKVRSVNSLMVASLTYKDKVYLNVTTDNTSLDDFHVNGGELILIH |
| by feeder cells | QNPGEFCVL |
| SEQ ID NO: 5 | RSKRSRLLHSDYMNMTPRRPGPTRKHYQPYAPPRDFAAYRS |
| CD28 intracellular | |
| signaling domain | |
| SEQ ID NO: 6 | AGGAGTAAGAGGAGCAGGCTCCTGCACAGTGACTACATGAACATGACTCCCCGCCGCC |
| CD28 intracellular | CCGGGCCCACCCGCAAGCATTACCAGCCCTATGCCCCACCACGCGACTTCGCAGCCTA |
| signaling domain | TCGCTCC |
| SEQ ID NO: 7 | CGGAGCAAGAGGTCCCGCCTGCTGCACAGCGACTATATGAACATGACCCCACGGAGAC |
| Codon Optimized | CCGGCCCTACACGGAAACATTACCAGCCCTATGCTCCACCCCGGGACTTCGCAGCTTA |
| CD28 intracellular | CAGAAGT |
| signaling domain | |
| SEQ ID NO: 8 | ERVQPLEENVGNAARPRFERNK |
| OX40L intracellular | |
| signaling domain | |
| SEQ ID NO: 9 | LEENVGNAARPRFERNK |
| OX40L intracellular | |
| signaling domain | |
| functional domain | |
| SEQ ID NO: 10 | RPRFERNK |
| OX40L intracellular | |
| signaling domain | |
| functional domain | |
| SEQ ID NO: 11 | GAAAGGGTCCAACCCCTGGAAGAGAATGTGGGAAATGCAGCCAGGCCAAGATTCGAGA |
| OX40L intracellular | GGAACAAG |
| signaling domain | |
| SEQ ID NO: 12 | GAAAGAGTGCAGCCCCTGGAAGAGAATGTCGGGAATGCCGCTCGCCCAAGATTTGAAA |
| Codon optimized | GGAACAAA |
| OX40L intracellular | |
| signaling domain | |
| SEQ ID NO: 13 | RVKFSRSADAPAYQQGQNQLYNELNLGRREEYDVLDKRRGRDPEMGGKPRRKNPQEGL |
| CD3ζ signaling | YNELQKDKMAEAYSEIGMKGERRRGKGHDGLYQGLSTATKDTYDALHMQALPPR |
| sequence | |
| SEQ ID NO: 14 | AGAGTGAAGTTCAGCAGGAGCGCAGACGCCCCCGCGTACCAGCAGGGCCAGAACCAGC |
| CD3ζ signaling | TCTATAACGAGCTCAATCTAGGACGAAGAGAGGAGTACGATGTTTTGGACAAGAGACG |
| sequence | TGGCCGGGACCCTGAGATGGGGGGAAAGCCGAGAAGGAAGAACCCTCAGGAAGGCCTG |
| | TACAATGAACTGCAGAAAGATAAGATGGCGGAGGCCTACAGTGAGATTGGGATGAAAG |
| | GCGAGCGCCGGAGGGGCAAGGGGCACGATGGCCTTTACCAGGGTCTCAGTACAGCCAC |
| | CAAGGACACCTACGACGCCCTTCACATGCAGGCCCTGCCCCCTCGC |
| SEQ ID NO: 15 | CGAGTGAAGTTCAGCAGGTCCGCCGACGCTCCTGCATACCAGCAGGGACAGAACCAGC |
| Codon optimized CD3ζ | TGTATAACGAGCTGAATCTGGGCCGGAGAGAGGAATACGACGTGCTGGACAAAAGGCG |
| signaling sequence | GGGCCGGGACCCCGAAATGGGAGGGAAGCCACGACGGAAAAACCCCCAGGAGGGCCTG |
| | TACAATGAGCTGCAAAAGGACAAAATGGCCGAGGCTTATTCTGAAATCGGGATGAAGG |
| | GAGAGAGAAGGCGCGGAAAAGGCCACGATGGCCTGTACCAGGGGCTGAGCACCGCTAC |
| | AAAGGACACCTATGATGCACTGCACATGCAGGCCCTGCCCCCTCGG |
| SEQ ID NO: 16 | GDVEXNPGP |
| 2A cleavage motif | |
| SEQ ID NO: 17 | GSGEGRGSLLTCGDVEENPGP |
| T2A cleavage site | |
| SEQ ID NO: 18 | GGCTCAGGTGAGGGGCGCGGGAGCCTGCTGACTTGTGGGGATGTAGAGGAAAATCCTG |
| T2A cleavage site | GTCCT |
| SEQ ID NO: 19 | GSGATNFSLLKQAGDVEENPGP |
| P2A cleavage site | |
| SEQ ID NO: 20 | GSGQCTNYALLKLAGDVESNPGP |
| E2A cleavage site | |
| SEQ ID NO: 21 | GSGVKQTLNFDLLKLAGDVESNPGP |
| F2A cleavage site | |
| SEQ ID NO: 22 | MRISKPHLRSISIQCYLCLLLNSHFLTEAGIHVFILGCFSAGLPKTEANWVNVISDLK |
| IL-15 | KIEDLIQSMHIDATLYTESDVHPSCKVTAMKCFLLELQVISLESGDASIHDTVENLII |
| | LANNSLSSNGNVTESGCKECEELEEKNIKEFLQSFVHIVQMFINTS- |
| SEQ ID NO: 23 | ATGAGAATCAGCAAACCACACCTCCGGAGCATATCAATCCAGTGTTACTTGTGCCTTC |
| IL-15 | TTTTGAACTCCCATTTCCTCACCGAGGCAGGCATTCATGTGTTCATATTGGGGTGCTT |
| | TAGTGCTGGGCTTCCGAAAACGGAAGCTAACTGGGTAAACGTCATCAGTGACCTTAAA |
| | AAAATTGAGGATCTTATCCAATCAATGCACATCGACGCGACTCTCTACACAGAATCTG |
| | ACGTACACCCGTCATGCAAAGTCACGGCAATGAAGTGTTTTCTTCTCGAGCTCCAAGT |
| | AATTTCCCTGGAGTCTGGCGATGCCTCCATCCACGATACGGTTGAAAATCTGATTATA |
| | TTGGCCAACAATAGCCTCAGTTCTAACGGTAACGTGACTGAAAGTGGCTGCAAAGAGT |
| | GCGAAGAGCTCGAAGAAAAGAATATCAAGGAGTTCCTCCAATCATTTGTTCACATTGT |
| | GCAAATGTTTATCAACACCTCTTGA |
| SEQ ID NO: 24 | ATGCGCATAAGTAAGCCTCATCTGCGGTCCATTTCTATACAATGTTATCTGTGCTTGC |
| IL-15 | TTTTGAACTCCCACTTTCTTACGGAAGCAGGCATTCATGTGTTCATTCTGGGTTGTTT |
| | TTCtGCCGGGCTGCCCAAAACCGAGGCCAACTGGGTCAACGTGATCAGCGACCTCAAG |
| | AAGATCGAGGATTTGATTCAAAGTATGCATATAGACGCCACACTCTATACTGAGTCCG |
| | ACGTTCACCCGAGTTGTAAAGTTACGGCTATGAAGTGCTTTTTGTTGGAACTCCAGGT |
| | GATTTCCCTTGAATCCGGCGATGCGAGCATCCACGATACGGTAGAGAATCTTATTATT |
| | CTGGCGAATAATTCTCTGTCTTCAAATGGGAATGTAACTGAGAGCGGTTGTAAAGAAT |
| | GCGAAGAACTTGAAGAAAAGAATATCAAGGAATTTCTTCAGAGTTTCGTGCATATTGT |
| | TCAAATGTTCATCAACACATCCTGA |
| SEQ ID NO: 25 | RSKRSRLLHSDYMNMTPRRPGPTRKHYQPYAPPRDFAAYRSERVQPLEENVGNAARPR |
| CD28/OX40L/CDζ | FERNKRVKFSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDALHMQALPP |
| | R |
| SEQ ID NO: 26 | RSKRSRLLHSDYMNMTPRRPGPTRKHYQPYAPPRDFAAYRSERVQPLEENVGNAARPR |
| CD28/OX40L/CDζ/T2 | FERNKRVKFSRSADAPAYQQGQNQLYNELNLGRREEYDVLDKRRGRDPEMGGKPRRKN |
| A/IL1-5 | PQEGLYNELQKDKMAEAYSEIGMKGERRRGKGHDGLYQGLSTATKDTYDALHMQALPP |
| | RGSGEGRGSLLTCGDVEENPGPMRISKPHLRSISIQCYLCLLLNSHELTEAGIHVFIL |
| | GCFSAGLPKTEANWVNVISDLKKIEDLIQSMHIDATLYTESDVHPSCKVTAMKCELLE |
| | LQVISLESGDASIHDTVENLIILANNSLSSNGNVTESGCKECEELEEKNIKEFLQSFV |
| | HIVQMFINTS- |
| SEQ ID NO: 27 | MALPVTALLLPLALLLHAARPDIQMTQSPSSLSASVGDRVTITCKASQDINSYLSWFQ |
| HER2 CAR with T2A | QKPGKAPKTLIYRANRLVDGVPSRFSGSGSGQDYTLTISSLQPEDFATYYCLQYDEFP |
| and IL-15 | WTFGQGTKVEIKGGGGSGGGGSGGGGSQVQLVQSGSELKKPGASVKVSCKASGYTFTN |
| | YGVNWVRQAPGQGLEWMGWINTHTGEPTYAEEFKGRFVFSLDTSVSTAYLQISSLKAE |
| | DTAVYYCARDDYYVRVDYWGQGTTVTVSSAKPTTTPAPRPPTPAPTIASQPLSLRPEA |
| | CRPAAGGAVHTRGLDFACDFWVLVVVGGVLACYSLLVTVAFIIFWVRSKRSRLLHSDY |
| | MNMTPRRPGPTRKHYQPYAPPRDFAAYRSERVQPLEENVGNAARPRFERNKRVKESRS |
| | ADAPAYQQGQNQLYNELNLGRREEYDVLDKRRGRDPEMGGKPRRKNPQEGLYNELQKD |
| | KMAEAYSEIGMKGERRRGKGHDGLYQGLSTATKDTYDALHMQALPPRGSGEGRGSLLT |
| | CGDVEENPGPMRISKPHLRSISIQCYLCLLLNSHFLTEAGIHVFILGCFSAGLPKTEA |
| | NWVNVISDLKKIEDLIQSMHIDATLYTESDVHPSCKVTAMKCFLLELQVISLESGDAS |
| | IHDTVENLIILANNSLSSNGNVTESGCKECEELEEKNIKEFLQSFVHIVQMFINTS- |
| SEQ ID NO: 28 | ATGGCACTTCCTGTTACAGCCCTCCTGCTCCCACTGGCTTTGCTGCTGCATGCTGCAC |
| Codon Optimized | GACCGGACATCCAGATGACCCAGAGCCCCAGCAGCCTGAGCGCCAGCGTGGGCGACAG |
| HER2 CAR with T2A | AGTGACCATCACCTGCAAGGCCAGCCAGGACATCAACAGCTACCTGAGCTGGTTCCAG |
| and IL-15 | CAGAAGCCCGGCAAGGCCCCCAAGACCCTGATCTACAGAGCCAACAGACTGGTGGACG |
| | GCGTGCCCAGCAGATTCAGCGGCAGCGGCAGCGGCCAGGACTACACCCTGACCATCAG |
| | CAGCCTGCAGCCCGAGGACTTCGCCACCTACTACTGCCTGCAGTACGACGAGTTCCCC |
| | TGGACCTTCGGCCAGGGCACCAAGGTGGAGATCAAGGGTGGCGGTGGATCGGGCGGTG |
| | GTGGATCTGGAGGAGGTGGCTCCCAGGTGCAGCTGGTGCAGAGCGGCAGCGAGCTGAA |
| | GAAGCCCGGCGCCAGCGTGAAGGTGAGCTGCAAGGCCAGCGGCTACACCTTCACCAAC |
| | TACGGCGTGAACTGGGTGAGACAGGCCCCCGGCCAGGGCCTGGAGTGGATGGGCTGGA |
| | TCAACACCCACACCGGCGAGCCCACCTACGCCGAGGAGTTCAAGGGCAGATTCGTGTT |
| | CAGCCTGGACACCAGCGTGAGCACCGCCTACCTGCAGATCAGCAGCCTGAAGGCCGAG |
| | GACACCGCCGTGTACTACTGCGCCAGAGACGACTACTACGTGAGAGTGGACTACTGGG |
| | GCCAGGGCACCACCGTGACCGTGAGCAGCGCAAAACCTACCACAACTCCTGCACCACG |
| | CCCCCCTACTCCAGCACCTACCATCGCATCTCAGCCACTGAGTCTGCGACCAGAGGCC |
| | TGCCGGCCCGCCGCCGGCGGGGCCGTCCATACCAGAGGGCTGGACTTTGCCTGCGATT |
| | TTTGGGTCCTGGTGGTCGTGGGAGGGGTGCTGGCATGTTACTCACTGCTGGTCACCGT |
| | GGCCTTCATCATCTTCTGGGTGCGGAGCAAGAGGTCCCGCCTGCTGCACAGCGACTAT |
| | ATGAACATGACCCCACGGAGACCCGGCCCTACACGGAAACATTACCAGCCCTATGCTC |
| | CACCCCGGGACTTCGCAGCTTACAGAAGTGAAAGAGTGCAGCCCCTGGAAGAGAATGT |
| | CGGGAATGCCGCTCGCCCAAGATTTGAAAGGAACAAACGAGTGAAGTTCAGCAGGTCC |
| | GCCGACGCTCCTGCATACCAGCAGGGACAGAACCAGCTGTATAACGAGCTGAATCTGG |
| | GCCGGAGAGAGGAATACGACGTGCTGGACAAAAGGCGGGGCCGGGACCCCGAAATGGG |
| | AGGGAAGCCACGACGGAAAAACCCCCAGGAGGGCCTGTACAATGAGCTGCAAAAGGAC |
| | AAAATGGCCGAGGCTTATTCTGAAATCGGGATGAAGGGAGAGAGAAGGCGCGGAAAAG |
| | GCCACGATGGCCTGTACCAGGGGCTGAGCACCGCTACAAAGGACACCTATGATGCACT |
| | GCACATGCAGGCCCTGCCCCCTCGGGGCTCAGGTGAGGGGCGCGGGAGCCTGCTGACT |
| | TGTGGGGATGTAGAGGAAAATCCTGGTCCTATGAGAATCAGCAAACCACACCTCCGGA |
| | GCATATCAATCCAGTGTTACTTGTGCCTTCTTTTGAACTCCCATTTCCTCACCGAGGC |
| | AGGCATTCATGTGTTCATATTGGGGTGCTTTAGTGCTGGGCTTCCGAAAACGGAAGCT |
| | AACTGGGTAAACGTCATCAGTGACCTTAAAAAAATTGAGGATCTTATCCAATCAATGC |
| | ACATCGACGCGACTCTCTACACAGAATCTGACGTACACCCGTCATGCAAAGTCACGGC |
| | AATGAAGTGTTTTCTTCTCGAGCTCCAAGTAATTTCCCTGGAGTCTGGCGATGCCTCC |
| | ATCCACGATACGGTTGAAAATCTGATTATATTGGCCAACAATAGCCTCAGTTCTAACG |
| | GTAACGTGACTGAAAGTGGCTGCAAAGAGTGCGAAGAGCTCGAAGAAAAGAATATCAA |
| | GGAGTTCCTCCAATCATTTGTTCACATTGTGCAAATGTTTATCAACACCTCT |
| SEQ ID NO: 29 | AKPTTTPAPRPPTPAPTIASQPLSLRPEACRPAAGGAVHTRGLDFACD |
| CD8α hinge | |
| SEQ ID NO: 30 | GCGAAGCCCACCACGACGCCAGCGCCGCGACCACCAACACCGGCGCCCACCATCGCGT |
| CD8α hinge | CGCAGCCCCTGTCCCTGCGCCCAGAGGCGTGCCGGCCAGCGGCGGGGGGCGCAGTGCA |
| | CACGAGGGGGCTGGACTTCGCCTGTGAT |
| SEQ ID NO: 31 | GCAAAACCTACCACAACTCCTGCACCACGCCCCCCTACTCCAGCACCTACCATCGCAT |
| Codon Optimized | CTCAGCCACTGAGTCTGCGACCAGAGGCCTGCCGGCCCGCCGCCGGCGGGGCCGTCCA |
| CD8α hinge | TACCAGAGGGCTGGACTTTGCCTGCGAT |
| SEQ ID NO: 32 | FWVLVVVGGVLACYSLLVTVAFIIFWV |
| CD28 transmembrane | |
| domain | |
| SEQ ID NO: 33 | TTTTGGGTGCTGGTGGTGGTTGGTGGAGTCCTGGCTTGCTATAGCTTGCTAGTAACAG |
| CD28 transmembrane | TGGCCTTTATTATTTTCTGGGTG |
| domain | |
| SEQ ID NO: 34 | TTTTGGGTCCTGGTGGTCGTGGGAGGGGTGCTGGCATGTTACTCACTGCTGGTCACCG |
| Codon Optimized | TGGCCTTCATCATCTTCTGGGTG |
| CD28 transmembrane | |
| domain | |
| SEQ ID NO: 35 | DIQMTQTTSSLSASLGDRVTISCRASQDISKYLNWYQQKPDGTVKLLIYHTSRLHSGV |
| CD19 CAR-MU-28 | PSRFSGSGSGTDYSLTISNLEQEDIATYFCQQGNTLPYTFGGGTKLEITGGGGSGGGG |
| | SGGGGSEVKLQESGPGLVAPSQSLSVTCTVSGVSLPDYGVSWIRQPPRKGLEWLGVIW |
| | GSETTYYNSALKSRLTIIKDNSKSQVFLKMNSLQTDDTAIYYCAKHYYYGGSYAMDYW |
| | GQGTSVTVSSEPKSCDKTHTCPPCPAPPVAGPSVFLFPPKPKDTLMISRTPEVTCVVV |
| | DVSHEDPEVKFNWYVDGVEVHNAKTKPREEQYQSTYRVVSVLTVLHQDWLNGKEYKCK |
| | VSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQ |
| | KSLSLSPGKFWVLVVVGGVLACYSLLVTVAFIIFWVRSKRSRLLHSDYMNMTPRRPGP |
| | TRKHYQPYAPPRDFAAYRSERVQPLEENVGNAARPRFERNKRVKFSRSADAPAYQQGQ |
| | NQLYNELNLGRREEYDVLDKRRGRDPEMGGKPRRKNPQEGLYNELQKDKMAEAYSEIG |
| | MKGERRRGKGHDGLYQGLSTATKDTYDALHMQALPPR |
| SEQ ID NO: 36 | MALPVTALLLPLALLLHAARPDIQMTQTTSSLSASLGDRVTISCRASQDISKYLNWYQ |
| CD19 CAR-MU-28 | QKPDGTVKLLIYHTSRLHSGVPSRFSGSGSGTDYSLTISNLEQEDIATYFCQQGNTLP |
| with T2A and IL-15 | YTFGGGTKLEITGGGGSGGGGSGGGGSEVKLQESGPGLVAPSQSLSVTCTVSGVSLPD |
| | YGVSWIRQPPRKGLEWLGVIWGSETTYYNSALKSRLTIIKDNSKSQVFLKMNSLQTDD |
| | TAIYYCAKHYYYGGSYAMDYWGQGTSVTVSSEPKSCDKTHTCPPCPAPPVAGPSVELF |
| | PPKPKDTLMISRTPEVTCVVVDVSHEDPEVKFNWYVDGVEVHNAKTKPREEQYQSTYR |
| | VVSVLTVLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLTVDKSRW |
| | QQGNVFSCSVMHEALHNHYTQKSLSLSPGKFWVLVVVGGVLACYSLLVTVAFIIFWVR |
| | SKRSRLLHSDYMNMTPRRPGPTRKHYQPYAPPRDFAAYRSERVQPLEENVGNAARPRF |
| | ERNKRVKFSRSADAPAYQQGQNQLYNELNLGRREEYDVLDKRRGRDPEMGGKPRRKNP |
| | QEGLYNELQKDKMAEAYSEIGMKGERRRGKGHDGLYQGLSTATKDTYDALHMQALPPR |
| | GSGEGRGSLLTCGDVEENPGPMRISKPHLRSISIQCYLCLLLNSHELTEAGIHVFILG |
| | CFSAGLPKTEANWVNVISDLKKIEDLIQSMHIDATLYTESDVHPSCKVTAMKCFLLEL |
| | QVISLESGDASIHDTVENLIILANNSLSSNGNVTESGCKECEELEEKNIKEFLQSFVH |
| | IVQMFINTS- |
| SEQ ID NO: 37 | DIQMTQTTSSLSASLGDRVTISCRASQDISKYLNWYQQKPDGTVKLLIYHTSRLHSGV |
| anti-CD19 scFv | PSRFSGSGSGTDYSLTISNLEQEDIATYFCQQGNTLPYTFGGGTKLEITGGGGGGGG |
| | SGGGGSEVKLQESGPGLVAPSQSLSVTCTVSGVSLPDYGVSWIRQPPRKGLEWLGVIW |
| | GSETTYYNSALKSRLTIIKDNSKSQVFLKMNSLQTDDTAIYYCAKHYYYGGSYAMDYW |
| | GQGTSVTVSS |
| SEQ ID NO: 38 | DIQMTQTTSSLSASLGDRVTISCRASQDISKYLNWYQQKPDGTVKLLIYHTSRLHSGV |
| CD19 CAR-MU-28 | PSRFSGSGSGTDYSLTISNLEQEDIATYFCQQGNTLPYTFGGGTKLEITGGGGSGGGG |
| | SGGGGSEVKLQESGPGLVAPSQSLSVTCTVSGVSLPDYGVSWIRQPPRKGLEWLGVIW |
| | GSETTYYNSALKSRLTIIKDNSKSQVFLKMNSLQTDDTAIYYCAKHYYYGGSYAMDYW |
| | GQGTSVTVSSEPKSCDKTHTCPPCPAPPVAGPSVFLFPPKPKDTLMISRTPEVTCVVV |
| | DVSHEDPEVKFNWYVDGVEVHNAKTKPREEQYQSTYRVVSVLTVLHQDWLNGKEYKCK |
| | VSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQ |
| | KSLSLSPGKFWVLVVVGGVLACYSLLVTVAFIIFWVRSKRSRLLHSDYMNMTPRRPGP |
| | TRKHYQPYAPPRDFAAYRSERVQPLEENVGNAARPRFERNKRVKFSRSADAPAYQQGQ |
| | NQLYNELNLGRREEYDVLDKRRGRDPEMGGKPRRKNPQEGLYNELQKDKMAEAYSEIG |
| | MKGERRRGKGHDGLYQGLSTATKDTYDALHMQALPPR |
| SEQ ID NO: 39 | DIQMTQTTSSLSASLGDRVTISCRASQDISKYLNWYQQKPDGTVKLLIYHTSRLHSGV |
| Anti-CD19 CAR | PSRFSGSGSGTDYSLTISNLEQEDIATYFCQQGNTLPYTFGGGTKLEITGGGGSGGGG |
| (CD28-CD3z-IL15) | SGGGGSEVKLQESGPGLVAPSQSLSVTCTVSGVSLPDYGVSWIRQPPRKGLEWLGVIW |
| | GSETTYYNSALKSRLTIIKDNSKSQVFLKMNSLQTDDTAIYYCAKHYYYGGSYAMDYW |
| | GQGTSVTVSSEPKSCDKTHTCPPCPAPPVAGPSVFLFPPKPKDTLMISRTPEVTCVVV |
| | DVSHEDPEVKFNWYVDGVEVHNAKTKPREEQYQSTYRVVSVLTVLHQDWLNGKEYKCK |
| | VSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQ |
| | KSLSLSPGKFWVLVVVGGVLACYSLLVTVAFIIFWVRSKRSRLLHSDYMNMTPRRPGP |
| | TRKHYQPYAPPRDFAAYRSRVKFSRSADAPAYQQGQNQLYNELNLGRREEYDVLDKRR |
| | GRDPEMGGKPRRKNPQEGLYNELQKDKMAEAYSEIGMKGERRRGKGHDGLYQGLSTAT |
| | KDTYDALHMQALPPR |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polynucleotide comprising:
a) a nucleic acid encoding a chimeric antigen receptor (CAR) comprising an intracellular signaling region comprising an OX40L intracellular signaling domain; and
b) a nucleic acid encoding an IL-15.

2. (canceled)

3. The polynucleotide of claim 1, wherein the OX40L intracellular signaling domain comprises an amino acid sequence set forth in SEQ ID NO: 8, SEQ ID NO: 9, or SEQ ID NO: 10.

4. The polynucleotide of claim 1, wherein the OX40L intracellular signaling domain comprises an amino acid sequence having at least 99% identity to SEQ ID NO: 8, SEQ ID NO: 9, or SEQ ID NO: 10.

5.-13. (canceled)

14. The polynucleotide of claim 1, wherein the IL-15 comprises the amino acid sequence set forth in SEQ ID NO: 22.

15. The polynucleotide of claim 1, wherein the IL-15 comprises an amino acid sequence having at least 99% identity to SEQ ID NO: 22.

16. The polynucleotide of claim 14, wherein the IL-15 is encoded by a nucleic acid comprising SEQ ID NO: 17 or SEQ ID NO: 18.

17. The polynucleotide of claim 1, wherein the polynucleotide encodes a polyprotein comprising the CAR and the IL-15.

18. The polynucleotide of claim 1, further comprising a nucleic acid encoding a self-cleaving peptide.

19. The polynucleotide of claim 18, wherein the CAR is joined to the IL-15 by the self-cleaving peptide.

20. The polynucleotide of claim 19, wherein the self-cleaving peptide is a T2A self-cleaving peptide.

21. (canceled)

22. The polynucleotide of claim 19, wherein the T2A self-cleaving peptide is capable of inducing ribosomal skipping between the CAR and the IL-15.

23.-38. (canceled)

39. A vector comprising the polynucleotide of claim 1.

40.-41. (canceled)

42. A cell comprising the polynucleotide of claim 1.

43. A cell expressing the chimeric antigen receptor and the IL-15 encoded by the polynucleotide of claim 1.

44. The cell of claim 42, wherein the cell is a lymphocyte.

45. The cell of claim 44, wherein the lymphocyte is a natural killer (NK) cell.

46.-51. (canceled)

52. A population of cells comprising a plurality of the cells of claim 42.

53.-54. (canceled)

55. A pharmaceutical composition comprising the population of cells of claim 52.

56.-72. (canceled)

73. A frozen vial comprising the composition of claim 55.

74. A method of treatment comprising administering the cell of claim 42 to a subject having a disease or condition.


SEQUENCE LISTING (39 sequences)

SEQ ID NO: 1 - protein, 254 aa, Artificial Sequence
  4-1BBL expressed by feeder cells
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 223 aa, Artificial Sequence
  mbIL-21 expressed by feeder cells
MALPVTALLLPLALLLHAARPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAKPTTTPAPRPPTPAPTIASQPLSLRPEACRPAAGGAVHTRGLDFACDIYIWAPLAGTCGVLLLSLVITLY

SEQ ID NO: 3 - protein, 233 aa, Artificial Sequence
  TNF alpha expressed by feeder cells
MSTESMIRDVELAEEALPKKTGGPQGSRRCLFLSLFSFLIVAGATTLFCLLHFGVIGPQREEFPRDLSLISPLAQPVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 183 aa, Artificial Sequence
  OX40L expressed by feeder cells
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - protein, 41 aa, Artificial Sequence
  CD28 intracellular signaling domain
RSKRSRLLHSDYMNMTPRRPGPTRKHYQPYAPPRDFAAYRS

SEQ ID NO: 6 - protein, 123 aa, Artificial Sequence
  CD28 intracellular signaling domain
AGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCC

SEQ ID NO: 7 - protein, 123 aa, Artificial Sequence
  Codon Optimized CD28 intracellular signaling domain
CGGAGCAAGAGGTCCCGCCTGCTGCACAGCGACTATATGAACATGACCCCACGGAGACCCGGCCCTACACGGAAACATTACCAGCCCTATGCTCCACCCCGGGACTTCGCAGCTTACAGAAGT

SEQ ID NO: 8 - protein, 22 aa, Artificial Sequence
  OX40L intracellular signaling domain
ERVQPLEENVGNAARPRFERNK

SEQ ID NO: 9 - protein, 17 aa, Artificial Sequence
  OX40L intracellular signaling domain functional domain
LEENVGNAARPRFERNK

SEQ ID NO: 10 - protein, 8 aa, Artificial Sequence
  OX40L intracellular signaling domain functional domain
RPRFERNK

SEQ ID NO: 11 - protein, 66 aa, Artificial Sequence
  OX40L intracellular signaling domain
GAAAGGGTCCAACCCCTGGAAGAGAATGTGGGAAATGCAGCCAGGCCAAGATTCGAGAGGAACAAG

SEQ ID NO: 12 - protein, 66 aa, Artificial Sequence
  Codon optimized OX40L intracellular signaling domain
GAAAGAGTGCAGCCCCTGGAAGAGAATGTCGGGAATGCCGCTCGCCCAAGATTTGAAAGGAACAAA

SEQ ID NO: 13 - protein, 112 aa, Artificial Sequence
  CD3 zeta signaling sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 336 aa, Artificial Sequence
  CD3 zeta signaling sequence
AGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGC

SEQ ID NO: 15 - protein, 336 aa, Artificial Sequence
  Codon optimized CD3 zeta signaling sequence
CGAGTGAAGTTCAGCAGGTCCGCCGACGCTCCTGCATACCAGCAGGGACAGAACCAGCTGTATAACGAGCTGAATCTGGGCCGGAGAGAGGAATACGACGTGCTGGACAAAAGGCGGGGCCGGGACCCCGAAATGGGAGGGAAGCCACGACGGAAAAACCCCCAGGAGGGCCTGTACAATGAGCTGCAAAAGGACAAAATGGCCGAGGCTTATTCTGAAATCGGGATGAAGGGAGAGAGAAGGCGCGGAAAAGGCCACGATGGCCTGTACCAGGGGCTGAGCACCGCTACAAAGGACACCTATGATGCACTGCACATGCAGGCCCTGCCCCCTCGG

SEQ ID NO: 16 - protein, 9 aa, Artificial Sequence
  2A cleavage motif
  MISC_FEATURE (5)..(5): x=any amino acid
GDVEXNPGP

SEQ ID NO: 17 - protein, 21 aa, Artificial Sequence
  T2A cleavage site
GSGEGRGSLLTCGDVEENPGP

SEQ ID NO: 18 - protein, 63 aa, Artificial Sequence
  T2A cleavage site
GGCTCAGGTGAGGGGCGCGGGAGCCTGCTGACTTGTGGGGATGTAGAGGAAAATCCTGGTCCT

SEQ ID NO: 19 - protein, 22 aa, Artificial Sequence
  P2A cleavage site
GSGATNFSLLKQAGDVEENPGP

SEQ ID NO: 20 - protein, 23 aa, Artificial Sequence
  E2A cleavage site
GSGQCTNYALLKLAGDVESNPGP

SEQ ID NO: 21 - protein, 25 aa, Artificial Sequence
  F2A cleavage site
GSGVKQTLNFDLLKLAGDVESNPGP

SEQ ID NO: 22 - protein, 162 aa, Artificial Sequence
  IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 489 aa, Artificial Sequence
  IL-15
ATGAGAATCAGCAAACCACACCTCCGGAGCATATCAATCCAGTGTTACTTGTGCCTTCTTTTGAACTCCCATTTCCTCACCGAGGCAGGCATTCATGTGTTCATATTGGGGTGCTTTAGTGCTGGGCTTCCGAAAACGGAAGCTAACTGGGTAAACGTCATCAGTGACCTTAAAAAAATTGAGGATCTTATCCAATCAATGCACATCGACGCGACTCTCTACACAGAATCTGACGTACACCCGTCATGCAAAGTCACGGCAATGAAGTGTTTTCTTCTCGAGCTCCAAGTAATTTCCCTGGAGTCTGGCGATGCCTCCATCCACGATACGGTTGAAAATCTGATTATATTGGCCAACAATAGCCTCAGTTCTAACGGTAACGTGACTGAAAGTGGCTGCAAAGAGTGCGAAGAGCTCGAAGAAAAGAATATCAAGGAGTTCCTCCAATCATTTGTTCACATTGTGCAAATGTTTATCAACACCTCTTGA

SEQ ID NO: 24 - protein, 489 aa, Artificial Sequence
  IL-15
ATGCGCATAAGTAAGCCTCATCTGCGGTCCATTTCTATACAATGTTATCTGTGCTTGCTTTTGAACTCCCACTTTCTTACGGAAGCAGGCATTCATGTGTTCATTCTGGGTTGTTTTTCTGCCGGGCTGCCCAAAACCGAGGCCAACTGGGTCAACGTGATCAGCGACCTCAAGAAGATCGAGGATTTGATTCAAAGTATGCATATAGACGCCACACTCTATACTGAGTCCGACGTTCACCCGAGTTGTAAAGTTACGGCTATGAAGTGCTTTTTGTTGGAACTCCAGGTGATTTCCCTTGAATCCGGCGATGCGAGCATCCACGATACGGTAGAGAATCTTATTATTCTGGCGAATAATTCTCTGTCTTCAAATGGGAATGTAACTGAGAGCGGTTGTAAAGAATGCGAAGAACTTGAAGAAAAGAATATCAAGGAATTTCTTCAGAGTTTCGTGCATATTGTTCAAATGTTCATCAACACATCCTGA

SEQ ID NO: 25 - protein, 175 aa, Artificial Sequence
  CD28/OX40L/CDzeta
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 358 aa, Artificial Sequence
  CD28/OX40L/CDzeta/T2A/IL1-5
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 27 - protein, 694 aa, Artificial Sequence
  HER2 CAR with T2A and IL-15
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 2082 aa, Artificial Sequence
  Codon Optimized HER2 CAR with T2A and IL-15
ATGGCACTTCCTGTTACAGCCCTCCTGCTCCCACTGGCTTTGCTGCTGCATGCTGCACGACCGGACATCCAGATGACCCAGAGCCCCAGCAGCCTGAGCGCCAGCGTGGGCGACAGAGTGACCATCACCTGCAAGGCCAGCCAGGACATCAACAGCTACCTGAGCTGGTTCCAGCAGAAGCCCGGCAAGGCCCCCAAGACCCTGATCTACAGAGCCAACAGACTGGTGGACGGCGTGCCCAGCAGATTCAGCGGCAGCGGCAGCGGCCAGGACTACACCCTGACCATCAGCAGCCTGCAGCCCGAGGACTTCGCCACCTACTACTGCCTGCAGTACGACGAGTTCCCCTGGACCTTCGGCCAGGGCACCAAGGTGGAGATCAAGGGTGGCGGTGGATCGGGCGGTGGTGGATCTGGAGGAGGTGGCTCCCAGGTGCAGCTGGTGCAGAGCGGCAGCGAGCTGAAGAAGCCCGGCGCCAGCGTGAAGGTGAGCTGCAAGGCCAGCGGCTACACCTTCACCAACTACGGCGTGAACTGGGTGAGACAGGCCCCCGGCCAGGGCCTGGAGTGGATGGGCTGGATCAACACCCACACCGGCGAGCCCACCTACGCCGAGGAGTTCAAGGGCAGATTCGTGTTCAGCCTGGACACCAGCGTGAGCACCGCCTACCTGCAGATCAGCAGCCTGAAGGCCGAGGACACCGCCGTGTACTACTGCGCCAGAGACGACTACTACGTGAGAGTGGACTACTGGGGCCAGGGCACCACCGTGACCGTGAGCAGCGCAAAACCTACCACAACTCCTGCACCACGCCCCCCTACTCCAGCACCTACCATCGCATCTCAGCCACTGAGTCTGCGACCAGAGGCCTGCCGGCCCGCCGCCGGCGGGGCCGTCCATACCAGAGGGCTGGACTTTGCCTGCGATTTTTGGGTCCTGGTGGTCGTGGGAGGGGTGCTGGCATGTTACTCACTGCTGGTCACCGTGGCCTTCATCATCTTCTGGGTGCGGAGCAAGAGGTCCCGCCTGCTGCACAGCGACTATATGAACATGACCCCACGGAGACCCGGCCCTACACGGAAACATTACCAGCCCTATGCTCCACCCCGGGACTTCGCAGCTTACAGAAGTGAAAGAGTGCAGCCCCTGGAAGAGAATGTCGGGAATGCCGCTCGCCCAAGATTTGAAAGGAACAAACGAGTGAAGTTCAGCAGGTCCGCCGACGCTCCTGCATACCAGCAGGGACAGAACCAGCTGTATAACGAGCTGAATCTGGGCCGGAGAGAGGAATACGACGTGCTGGACAAAAGGCGGGGCCGGGACCCCGAAATGGGAGGGAAGCCACGACGGAAAAACCCCCAGGAGGGCCTGTACAATGAGCTGCAAAAGGACAAAATGGCCGAGGCTTATTCTGAAATCGGGATGAAGGGAGAGAGAAGGCGCGGAAAAGGCCACGATGGCCTGTACCAGGGGCTGAGCACCGCTACAAAGGACACCTATGATGCACTGCACATGCAGGCCCTGCCCCCTCGGGGCTCAGGTGAGGGGCGCGGGAGCCTGCTGACTTGTGGGGATGTAGAGGAAAATCCTGGTCCTATGAGAATCAGCAAACCACACCTCCGGAGCATATCAATCCAGTGTTACTTGTGCCTTCTTTTGAACTCCCATTTCCTCACCGAGGCAGGCATTCATGTGTTCATATTGGGGTGCTTTAGTGCTGGGCTTCCGAAAACGGAAGCTAACTGGGTAAACGTCATCAGTGACCTTAAAAAAATTGAGGATCTTATCCAATCAATGCACATCGACGCGACTCTCTACACAGAATCTGACGTACACCCGTCATGCAAAGTCACGGCAATGAAGTGTTTTCTTCTCGAGCTCCAAGTAATTTCCCTGGAGTCTGGCGATGCCTCCATCCACGATACGGTTGAAAATCTGATTATATTGGCCAACAATAGCCTCAGTTCTAACGGTAACGTGACTGAAAGTGGCTGCAAAGAGTGCGAAGAGCTCGAAGAAAAGAATATCAAGGAGTTCCTCCAATCATTTGTTCACATTGTGCAAATGTTTATCAACACCTCT

SEQ ID NO: 29 - protein, 48 aa, Artificial Sequence
  CD8 alpha hinge
AKPTTTPAPRPPTPAPTIASQPLSLRPEACRPAAGGAVHTRGLDFACD

SEQ ID NO: 30 - protein, 144 aa, Artificial Sequence
  CD8 alpha hinge
GCGAAGCCCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGAT

SEQ ID NO: 31 - protein, 144 aa, Artificial Sequence
  Codon Optimized CD8 alpha hinge
GCAAAACCTACCACAACTCCTGCACCACGCCCCCCTACTCCAGCACCTACCATCGCATCTCAGCCACTGAGTCTGCGACCAGAGGCCTGCCGGCCCGCCGCCGGCGGGGCCGTCCATACCAGAGGGCTGGACTTTGCCTGCGAT

SEQ ID NO: 32 - protein, 27 aa, Artificial Sequence
  CD28 transmembrane domain
FWVLVVVGGVLACYSLLVTVAFIIFWV

SEQ ID NO: 33 - protein, 81 aa, Artificial Sequence
  CD28 transmembrane domain
TTTTGGGTGCTGGTGGTGGTTGGTGGAGTCCTGGCTTGCTATAGCTTGCTAGTAACAGTGGCCTTTATTATTTTCTGGGTG

SEQ ID NO: 34 - protein, 81 aa, Artificial Sequence
  Codon Optimized CD28 transmembrane domain
TTTTGGGTCCTGGTGGTCGTGGGAGGGGTGCTGGCATGTTACTCACTGCTGGTCACCGTGGCCTTCATCATCTTCTGGGTG

SEQ ID NO: 35 - protein, 675 aa, Artificial Sequence
  CD19 CAR-MU-28
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 879 aa, Artificial Sequence
  CD19 CAR-MU-28 with T2A and IL-15
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - protein, 242 aa, Artificial Sequence
  anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 38 - protein, 675 aa, Artificial Sequence; identical to SEQ ID NO: 35
  CD19 CAR-MU-28

SEQ ID NO: 39 - protein, 653 aa, Artificial Sequence
  Anti-CD19 CAR (CD28-CD3z-IL15)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR